SEQUENCE LISTING (18 sequences)

SEQ ID NO: 1 - DNA, 18 bp, Artificial Sequence
  Synthetic construct
cttcccgtgtcagggatt

SEQ ID NO: 2 - DNA, 21 bp, Artificial Sequence
  Synthetic construct
tcacagttgggcaataagatg

SEQ ID NO: 3 - DNA, 24 bp, Artificial Sequence
  Synthetic construct
agccacagcttgagaagcaccaga

SEQ ID NO: 4 - DNA, 20 bp, Artificial Sequence
  Synthetic construct
tcaggtgtcatcccaagtgt

SEQ ID NO: 5 - DNA, 19 bp, Artificial Sequence
  Synthetic construct
gacaagctccagggagtga

SEQ ID NO: 6 - DNA, 26 bp, Artificial Sequence
  Synthetic construct
ggtaggtatagagcagctggggccag

SEQ ID NO: 7 - DNA, 21 bp, Artificial Sequence
  Synthetic construct
ggcctgtcctgagtctgaata

SEQ ID NO: 8 - DNA, 24 bp, Artificial Sequence
  Synthetic construct
agtcaagtgaattatccagtgctt

SEQ ID NO: 9 - DNA, 24 bp, Artificial Sequence
  Synthetic construct
ctttccatgtactgggctgctccg

SEQ ID NO: 10 - DNA, 20 bp, Artificial Sequence
  Synthetic construct
atccgcaagcctgtgactgt

SEQ ID NO: 11 - DNA, 18 bp, Artificial Sequence
  Synthetic construct
tcgggccagggtgttttt

SEQ ID NO: 12 - DNA, 18 bp, Artificial Sequence
  Synthetic construct
ttcccgggctcgttgccg

SEQ ID NO: 13 - DNA, 20 bp, Artificial Sequence
  Synthetic construct
ctctgctcctcctgttcgac

SEQ ID NO: 14 - DNA, 20 bp, Artificial Sequence
  Synthetic construct
acgaccaaatccgttgactc

SEQ ID NO: 15 - DNA, 18 bp, Artificial Sequence
  Synthetic construct
cggctgaccctgaaagag

SEQ ID NO: 16 - DNA, 18 bp, Artificial Sequence
  Synthetic construct
cagcccttgacagccttc

SEQ ID NO: 17 - DNA, 20 bp, Artificial Sequence
  Synthetic construct
tccagatccagcaaatagca

SEQ ID NO: 18 - DNA, 18 bp, Artificial Sequence
  Synthetic construct
gtccaagggcagctgtgt

(19) United States
(12) Patent Application Publication
(10) Pub. No.: US 2010/0297127 A1
(43) Pub. Date: Nov. 25, 2010

(54) USE OF IL-27 ANTAGONISTS TO TREAT LUPUS

(76) Inventors: Nico P. GHILARDI, Millbrae, CA (US); Marcel L. BATTEN, Enmore (AU); Jason A. HACKNEY, Palo Alto, CA (US)

Correspondence Address: MORRISON & FOERSTER LLP, 755 PAGE MILL RD, PALO ALTO, CA, 94304-1018, US

(21) Appl. No.: 12/756,871

(22) Filed: Apr. 8, 2010

Related U.S. Application Data
(60) Provisional application No. 61/167,793, filed on Apr. 8, 2009.
(60) Provisional application No. 61/267,185, filed on Dec. 7, 2009.

(51) Int. Cl. A61K 39/395; A61K 38/02; A61K 31/713; A61P 37/00; C12Q 1/68

(52) U.S. Cl. 424/136.1; 424/158.1; 424/145.1; 424/133.1; 514/1.1; 514/44 A; 435/6

(57) ABSTRACT

This invention relates to methods of treating the autoimmune disorder lupus with IL-27 antagonists, as well as articles of manufacture comprising IL-27 antagonists. The invention also relates to methods and kits for identifying patients that are likely to respond to an IL-27 antagonist treatment.

### CROSS-REFERENCE TO RELATED APPLICATIONS

This application claims the priority benefit of U. S. provisional application Ser. No. 61/167,793, filed Apr. 8, 2009, and Ser. No. 61/267,185, filed Dec. 7, 2009, all of which are incorporated herein by reference in their entirety.

### FIELD OF THE INVENTION

This invention relates to methods and compositions that modulate immune function. More specifically, the invention relates to compositions and methods for using IL-27 antagonists to treat lupus.

### BACKGROUND OF THE INVENTION

The cytokine interleukin-27 (“IL-27”) plays an important role in immune suppression, restricting inflammation in response to a wide variety of immune challenges. IL-27 is a relatively newly identified member of the IL-12 family of cytokines. This group of interleukins also includes the well known cytokine IL-12, a key T_{H}1 effector cytokine, as well as proinflammatory cytokines IL-6 and IL-23, which are important for the differentiation and expansion of T_{H}17 cells. IL-27 is a heterodimeric cytokine consisting of a classical 4-helix cytokine subunit protein p28 (“IL-27p28”) most closely resembling IL-12p35, and Epstein-Barr virus-induced protein 3 (“IL-27Ebi3”), a soluble cytokine receptor-like molecule similar to IL-12p40 and the IL-6 receptor.

IL-27 signals through a heterodimeric IL-27 receptor. M. Batten and N. Ghilardi, J. Mol. Med. 85(7):661-772 (2007). The heterodimeric IL-27 receptor contains a proprietary receptor chain designated IL-27Ra (also known as “WSX-1” or “TCCR”) and the IL-6 receptor β-chain designated gp130, which is also utilized by a number of other cytokines. Despite these similarities, IL-27 functions are distinct from its family members and both pro- and anti-inflammatory effects have been described. R. A. Kastelein et al., Ann. Rev. Immunol. 25:221-42 (2005); Batten and Ghilardi, J. Mol. Med. 85(7):661-772 (2007). IL-27 is generally cited as the product of monocytes and dendritic cells, where it is expressed after activation of Toll-like receptors (“TLRs”) in a MyD88 and NFκB-dependent way [S. Goriely et al., Nat. Rev. Immunol. 8(1):81-86 (2008)], however, it can also be expressed by B-cells. M. Hasan et al., Immunol. 123(2):239-49 (2008). The IL-27 receptor is expressed by most immune cells but the best described effects of IL-27 are those exerted on CD4+ T-cells. Kastelein et al., Ann. Rev. Immunol. 25:221-42 (2005); Batten and Ghilardi, J. Mol. Med. 85(7):661-772 (2007).

Although IL-27 promotes T_{H}1 responses in vitro by inducing the expression of T-bet and IFNγ [S. Lucas et al., Proc. Nat'l Acad. Sci. USA 100(25):15047-052 (2003); L. Hibbert et al., J. Interfer. Cytokine Res. 23(9):513-22 (2003); and A. Takeda et al., J. Immunol. 170(10):4886-90 (2003)], it is thought that IL-27 is predominantly an immunosuppressive cytokine, even restricting T_{H}1 responses in vivo. Mice with disrupted IL-27 signaling (IL-27Ra−/−, p28−/− and Ebi3−/−), display elevated immune responses to infections like peritonitis, Mycobacterium tuberculosis, Toxoplasma gondii and Trichuris muris. Id. Such mice also develop more severe pathology in models of Canavilin A-induced hepatitis, experimental autoimmune encephalomyelitis (EAE), and allergic asthma. Kastelein et al., Ann. Rev. Immunol. 25:221-42 (2005); Batten and Ghilardi, J. Mol. Med. 85(7):661-772 (2007). Furthermore, IL-27 directly suppresses T_{H}2 and T_{H}17 differentiation while inducing IL-10 production in vivo. S. Lucas et al., Proc. Nat'l Acad. Sci. USA 100(25):15047-052 (2003); M. Batten et al., J. Immunol. 180(5):2752-56 (2008); M. Batten et al., Nat. Immunol. 7(9):929-36 (2006); J. S. Stumhofer et al., Nat. Immunol. 7(9):937-45 (2006); J. S. Stumhofer et al., Nat. Immunol. 8(12):1363-71 (2007). It is thought that the induction of IL-10 activity is central to its generalized immunosuppressive activity. IL-27 also induces production of IL-21, a cytokine essential to the germinal center (GC) reaction.

In contrast, mice that lack IL-27 signaling are actually resistant to several autoimmune disease models, suggesting that IL-27 may have an activating role during some types of immune responses. Further evidence suggests that IL-27 may be essential for the proper functioning of germinal centers (GCs).

The GC is a temporary structure within secondary lymphoid organs in which hypermutating B-cells are positively selected for increased affinity and negatively selected against autoreactivity by competition for antigen presented on FDC and by acquisition of T-cell “help”. Affinity maturation of antibody producing B-cells and the development of B-cell memory are dependent on the GC reaction. C. D. Allen et al., Immunity 27(2):190-202 (2007). In accordance with its central role in humoral immunity, dysregulation of GCs is associated with reduced protective immunity to foreign organisms and the development of autoimmune disease. In systemic lupus erythematosus (“SLE”) patients, for example, self-reactive B-cells expressing the V_{H}4-34 heavy chain that are normally excluded from the GC can survive and differentiate into autoantibody secreting plasma and memory cells. A. E. Pugh-Bernard et al., J. Clin. Invest. 108(7):1061-70 (2001). Moreover, genetic mutations associated with increased GC activity have been shown to result in autoimmune disease in mouse models. F. Mackay et al., J. Exp. Med. 190(11):1697-710 (1999); C. G. Vinuesa et al., Nature 435(7041):452-58 (2008); U. Wellmann et al., Eur. J. Immunol. 31(9):2800-810 (2001).

T-cells participating in the GC reaction comprise a specialized subset of CD4+ cells termed “T follicular helper” (“T_{FH}”) cells, which can migrate into the B-cell follicle by virtue of the fact that they express CXCR5 and move towards the gradient of CXCL13 expression at this site. They are also characterized by high expression of the B-cell activating co-stimulatory molecule ICOS, CD40L, negative costimulatory molecule PD-1, the transcription factor Bcl6 and the cytokines IL-21 and IL-10 (C. King et al., Ann. Rev. Immunol. 26:741-66 (2008)), which promote B-cell proliferation, antibody isotype switching and differentiation. The integration of multiple costimulatory signals appears to be important for the generation of T_{FH} cells. Dysregulated T_{FH} activity in mutant mice leads to spontaneous development of multiple GCs and to a lupus-like autoimmune disease. The Sanroque mouse line, for example, has a homozygous point mutation in the roquin gene, which normally limits ICOS expression by promoting the degradation of ICOS messenger RNA. D. Yu et al., Nature 450(7167):299-303 (2007). Consequently, these mice display increased ICOS expression on T-cells and excessive T_{FH} cell differentiation which, in turn, leads to a T_{FH}-driven lupus-like autoimmune syndrome. C. G. Vinuesa et al., Nature 435(7041):452-58 (2008); and D. Yu et al., Nature 450(7167):299-303 (2007).

The factors governing the generation of T_{FH} cells are still only partially understood. IL-21 production is critical to the GC reaction. In addition, not only does IL-21 support B-cell proliferation and antibody production, it is also important for the T_{FH} cells themselves. The normal generation of T_{FH} cells appears to require a number of costimulatory signals that include IL-21 signaling through its receptor on CD4+ T helper cells at the T:B border. Transfer of IL-21R-sufficient CD4+ T-cells into IL-21R-deficient animals revealed that a T-cell intrinsic defect underpinned the limited GC formation and poor IgG1 response observed in the absence of IL-21:IL-21R signaling. A. Vogelzang et al., Immunity 29(1):127-37 (2008). Furthermore, IL-21 stimulation induced a T_{FH}-like transcriptional profile in CD4+ T-cells and supported the survival and proliferation of T_{FH} cells ex vivo. R. I. Nurieva et al., Immunity 29(1):138-49 (2008). In addition, it has been shown that wild-type T-cells could promote antibody production by IL-21R−/− B-cells but that IL-21R−/− T-cells had impaired capacity to help wild-type B-cells. F. Eddahri et al., Blood 113(11):2426-33 (2009). Taken together, these data indicate that autocrine IL-21 signaling to T-cells is essential for T_{FH} cells and the GC reaction as a whole.

The differentiation of GC B and T_{FH} T-cells appears to depend on the carefully co-ordinated bi-directional “crosstalk” between the T and B-cells. C. D. Allen et al., Immunity 27(2):190-202 (2007). Upon antigen recognition, B and T-cells move towards the T-B-cell border, at which point the receipt of B-cell-derived signals appears to be critical for the development of T_{FH} cells, as demonstrated by the severe reduction of CD4+CXCR5+T_{FH} cells in the B-cell-specific ICOSL mutant mice. R. I. Nurieva et al., Immunity 29(1):138-49 (2008). The T-B-cell interactions are therefore mutually beneficial, with T-cells providing proinflammatory signals such as CD40L and cytokines such as IL-4, IL-10 and IL-21. IL-27 production by GC B-cells may support the survival of T_{FH} cells as well as production of T_{FH} cytokines IL-10 [M. Batten et al., J. Immunol. 180(5):2752-56 (2008); and J. S. Stumhofer et al., Nat. Immunol. 8(12):1363-71 (2007)] and IL-21.

The ability of IL-27 to suppress immune responses on the one hand and to enhance T_{FH} cell survival and GC reactions on the other hand are not necessarily incompatible. Indeed, the ability of IL-27 to induce IL-10 production helps to reconcile these two observations. T_{FH} cells have been reported to suppress the activation of conventional CD4(+) T-cells via a direct contact-dependent mechanism as well as by releasing soluble mediators including IL-10, while at the same time providing critical help signals for B-cell response. E. Marinova et al., J. Immunol. 178(8):5010-17 (2007). This observation is consistent with the fact that IL-27 induces IL-10 production and also with induction of IL-21, another factor known to enhance IL-10 production. Spolski, R., et al., J. Immunol. 182(5):2859-67 (2009). In fact, it might be fundamentally important that T-cell suppressive mechanisms exist in the GC since it is dedicated to maturation of the B-cell response. In addition, IL-27 production by B-cells might represent a previously unappreciated mechanism of immunosuppression, which would explain the observation that IL-27 is immunosuppressive in many scenarios. These data suggest that IL-27 is important for T-dependent antibody maturation by enhancing the survival of T_{FH} cells, possibly via upregulation of IL-21. In doing so, IL-27 enhances IL-10 production, perhaps explaining why IL-27 dampens immune responses in many autoimmune disease models where high affinity antibody is not critical to disease, such as EAE.

All references cited herein, including patent applications and publications, are hereby incorporated by reference in their entirety.

### SUMMARY OF THE INVENTION

The invention provides methods for treating or preventing lupus (such as systemic lupus erythematosus (“SLE”)) in an individual comprising administering to the individual an effective amount of an IL-27 antagonist. In certain embodiments, the individual is a human. In certain embodiments, the individual has lupus or is at risk of developing lupus.

In certain embodiments, the IL-27 antagonist inhibits IL-27 signal transduction. In certain embodiments, the IL-27 antagonist inhibits the production of IL-10 (for example, IL-27-induced IL-10 production). In certain embodiments, the IL-27 antagonist inhibits the production of IL-21 (for example, IL-27-induced IL-21 production). In certain embodiments, the IL-27 antagonist reduces the number of T follicular helper cells. In certain embodiments, the IL-27 antagonist reduces the amount of high affinity antigen-specific antibodies.

In certain embodiments, the IL-27 antagonist is an anti-IL-27 antibody that specifically binds to IL-27. In certain embodiments, the IL-27 antagonist is an antibody that specifically binds to the Epstein Barr virus induced protein 3 (“Ebi3”) subunit of IL-27 (“IL-27Ebi3”). In certain embodiments, the anti-IL-27Ebi3 antibody specifically binds to the Ebi3 subunit of IL-27 and blocks its dimerization with the p28 subunit of IL-27. In certain embodiments, the IL-27 antagonist is an antibody specifically binds to the p28 subunit of IL-27 (“IL-27p28”). In certain embodiments, the anti-IL-27p28 antibody specifically binds to the p28 subunit of IL-27 and blocks its dimerization with the Ebi3 subunit of IL-27.

In certain embodiments, the IL-27 antagonist is an anti-IL-27 receptor antibody that specifically binds to IL-27Ra.

In certain embodiments, the antibodies described herein are monoclonal antibodies. In certain other embodiments, the antibodies are antibody fragments selected from the group consisting of Fab, Fab′-SH, Fv, scFv, and (Fab′)₂ fragments. In certain embodiments, the antibodies are humanized antibodies. In certain embodiments, the antibodies are human antibodies.

In certain embodiments, the IL-27 antagonist is a small molecule that inhibits binding between IL-27 and its receptor. In certain embodiments, the IL-27 antagonist is a polypeptide that inhibits binding between IL-27 and its receptor. In certain embodiments, the IL-27 antagonist is a short interfering RNA (“siRNA”) that inhibits expression of one or both subunits of IL-27, or IL-27Ra. In certain embodiments, the IL-27 antagonist is an RNA or DNA aptamer that binds to IL-27, one or both subunits of IL-27, or to IL-27Ra.

In certain embodiments, the IL-27 antagonist is administered intravenously, intramuscularly, subcutaneously, topically, orally, transdermally, intraperitoneally, intraorbitally, by implantation, by inhalation, intrathecally, intraventricularly, or intranasally. In certain embodiments, the IL-27 antagonist is used for treating or preventing lupus (such as SLE).

In certain embodiments, the individual has increased expression of one or more marker genes shown in FIG. 19A in peripheral blood mononuclear cells (PBMCs) from the individual as compared to a reference level. In certain embodiments, the individual has increased expression of at least 2, 3, 4, 5, 6, 7, 8, 9, 10, 11, 12, 13, 14, 15, 16, 17, 18, 19, 20, 21 or any number up to all of the marker genes shown in FIG. 19A in PBMCs from the individual as compared to the reference level of the respective marker genes. In certain embodiments, the expression of any one or more marker genes is measured at the level of an RNA transcript or at the level of a protein expression. In certain embodiments, the reference level is determined based on the expression level of the marker gene in PBMCs from one or more healthy individuals. In certain embodiments, the individual with lupus has a mean z-score greater than a mean z-score plus two standard deviations of the healthy individuals. The mean z-score may be calculated from the expression level of at least 2, 3, 4, 5, 6, 7, 8, 9, 10, 11, 12, 13, 14, 15, 16, 17, 18, 19, 20, 21 or any number up to all of the marker genes shown in FIG. 19A in PBMCs from the individual with lupus and healthy individuals.

The invention also provides a pharmaceutical composition comprising an IL-27 antagonist for use in treating or preventing lupus (such as SLE). The invention also provides use of an IL-27 antagonist in the manufacture of a medicament for treating or preventing lupus (such as SLE).

The invention also provides an article of manufacture comprising an IL-27 antagonist. In certain embodiments, the article further comprises instructions for using the IL-27 antagonist to treat or prevent lupus (such as SLE). In certain embodiments, the article further comprises a label or a package insert indicating that the IL-27 antagonist is for treating patients with lupus having increased expression of one or more marker genes shown in FIG. 19A in peripheral blood mononuclear cells (PBMCs) from the patients as compared to a reference level.

The invention also provide's a method for determining if a patient having lupus is likely to respond to an IL-27 antagonist treatment, comprising the steps of: (a) measuring the expression level of a marker gene shown in FIG. 19A in a sample comprising peripheral blood mononuclear cells (PBMCs) obtained from the patient; and (b) comparing the expression level measured in step (a) to a reference level, wherein an increase in the expression level of the marker gene as compared to the reference level indicates that the individual is likely to respond to the IL-27 antagonist treatment. In certain embodiments, the expression level of at least 2, 3, 4, 5, 6, 7, 8, 9, 10, 11, 12, 13, 14, 15, 16, 17, 18, 19, 20, 21 or any number up to all of the marker genes shown in FIG. 19A is measured and compared to the reference level of the respective genes. In certain embodiments, the expression level of all marker genes shown in FIG. 19A is measured and compared to the reference level of the respective genes. In certain embodiments, the expression level is measured at the level of an RNA transcript or at the level of a protein expression. In certain embodiments, the reference level is determined based on the expression level of the marker gene in PBMCs from one or more healthy individuals.

The invention also provides a method of preparing an expression profile for a patient having lupus, comprising the steps of: (a) measuring the expression level of a marker gene shown in FIG. 19A in a sample comprising peripheral blood mononuclear cells (PBMCs) obtained from the patient; and (b) generating a report summarizing the expression level measured in step (a). In certain embodiments, the method further comprises comparing the expression level of the marker gene measured in step (a) to a reference level; and generating a report summarizing the comparison. In certain embodiments, the expression level of at least 2, 3, 4, 5, 6, 7, 8, 9, 10, 11, 12, 13, 14, 15, 16, 17, 18, 19, 20, 21 or any number up to all of the marker genes shown in FIG. 19A is measured and/or compared to the reference level of the respective genes. In certain embodiments, the expression level of all marker genes shown in FIG. 19A are measured and/or compared to the reference level of the respective genes. In certain embodiments, the expression level is measured at the level of an RNA transcript or at the level of a protein expression. In certain embodiments, the reference level is determined based on the expression level of the marker gene in PBMCs from one or more healthy individuals. In certain embodiments, the report includes a recommendation for an IL-27 antagonist treatment for the patient.

The invention also provides kits comprising reagents for measuring the expression level of at least one of the marker genes shown in FIG. 19A in a sample comprising PBMCs from an individual having lupus. In certain embodiments, the kit further comprises instructions for assessing if the individual having lupus is likely to respond to an IL-27 antagonist treatment. In certain embodiments, the reagents comprise polynucleotides capable of specifically hybridizing to one or more marker genes shown in FIG. 19A or complements of said genes. In certain embodiments, the polynucleotides are capable of specifically hybridizing to at least 2, 3, 4, 5, 6, 7, 8, 9, 10, 11, 12, 13, 14, 15, 16, 17, 18, 19, 20, 21 or any number up to all of the marker genes marker genes shown in FIG. 19A or complements of said genes. In certain embodiments, the polynucleotides are provided as an array, a gene chip, or gene set. In certain embodiments, the reagents comprise at least a pair of primers and a probe for determining the expression level of a marker gene by PCR.

### BRIEF DESCRIPTION OF THE DRAWINGS

FIG. 1 shows the expression level of IL-27p28 (A), IL-21 (B), and IL-27Ra and gp130 (C) in various cell types present in the spleen as determined by quantitative RT-PCR. The cell types tested are non-GC B cells (non GC B), GC B-cells (GC B), follicular dendritic cells (FDC), T follicular helper cells (TFH), CD11b+ cells (CD11b), CD11c+ cells (CD11c), CD11b+ and CD11c+ cells (CD11c+b), total splenocytes (splenos), and CD4+ cells (CD4+).

FIG. 2 shows that IL-27 induces expression of IL-21 and IL-10 protein by anti-CD3/anti-CD28 stimulated CD4+ T cells, that IL-21 levels rise before those of IL-10, and confirms that IL-21 is also highly expressed under T_{H}17 conditions. FIG. 2A shows the concentration of IL-21 in the supernatant of purified CD4+ T-cells in the presence or absence of IL-27 measured at various time points by ELISA. FIG. 2B shows the concentration of IL-10 in the supernatant of purified CD4+ T-cells in the presence or absence of IL-27. FIG. 2C shows the concentration of IL-21 in the supernatant of purified CD4+ T-cells in the presence (open bars) or absence (closed bars) of IL-27 measured at various time points by ELISA. Error bars indicate SD of duplicates.

FIG. 2D shows the IL-21 mRNA level in CD4+ T-cells isolated from spleens of IL-27Ra+/+ mice (closed symbols) or IL-27Ra−/− (open symbols) mice at 4 or 8 days post-immunization with 30 μg TNP-OVA emulsified in CFA. FIG. 2E shows IL-27 induced IL-10 production (first and second plot), and the induction was reduced in the presence of a soluble IL-21R-Fc which blocked IL-21 signaling (third FACS plot).

FIG. 3 shows that GC area and the production of high affinity antibodies are reduced in spleens from IL-27Ra−/− mice. FIG. 3A shows the GC area in IL-27Ra+/+ mice and IL-27Ra−/− mice stained with PNA. FIG. 3B shows the electronic quantitation of PNA+ GC area in the spleens of eight IL-27Ra+/+ mice (WT) and eight IL-27Ra−/− mice. FIG. 3C shows relative concentrations of high affinity IgG antibodies in IL-27Ra+/+ mice (closed symbols) and IL-27Ra−/− mice (open symbols) after immunization. FIG. 3D shows concentrations of high affinity antibody of different isotype antibodies, IgE, IgM, IgG1, IgG2a, and IgG2b in IL-27Ra+/+ mice (closed symbols) and IL-27Ra−/− mice (open symbols) after immunization.

FIG. 4 shows that mice deficient in IL-27Ra have fewer T_{FH} cells than wild-type mice. IL-27Ra+/+ and −/− mice were immunized with 30 μg TNP-OVA in CFA (T-dependent; FIG. 4A) or TNP-Ficoll (T-independent) or left unchallenged. (A) Spleen was isolated 7 days later and stained with antibodies against CD4, B220, CXCR5, and ICOS or PD1. Cd4+B220− cells are shown and the cells with a T_{FH} cell phenotype are gated. (B) Average percentage of CXCR5+ICOS+ cells in the CD4+B220− gate of unimmunized mice or mice immunized with T-dependent or T-independent antigens.

FIG. 5 shows that IL-27 supports survival of T_{FH} cells. (A) DO11.10 TCR Tg CD4+ T-cells were stimulated with increasing concentrations of OVA peptide in the presence or absence of 20 ng/ml rmIL-27 for 72 hours in culture. (B) Annexin V and 7AAD viability staining on IL-27Ra+/+ and −/−cells stimulated with 0.03 μM OVA peptide in the presence and absence of 20 ng/ml rmIL-27. Viable non-apoptotic cells are 7AAD and AnV negative. (C) AnV and 7AAD staining of CXCR5+PD1+CD4+ TFH cells from IL-27Ra+/+ and IL-27Ra−/− mice immunized with 30 μg TNP-OVA in CFA. (D) Average T_{FH} cell viability in 8 mice as in FIG. 5C are shown plus and minus SEM.

FIG. 6 shows that IL-27 is critical for supporting T_{FH} cells in vivo. Wild-type (WT) and IL-27Ra−/− mice (KO) were immunized with TNP-OVA in CFA and spleens were harvested four or eight days later. FIG. 6A shows the average percentage of CXCR5+/ICOS+CD4+ T-cells. FIG. 6B shows the average percentage of CXCR5+/PD1+CD4+ T-cells.

FIG. 7 shows the results of principal component analysis confirming an IL-27 gene signature that permits discrimination between healthy patients and patients with SLE in cohort 1 (FIG. 7A) and cohort 2 (FIG. 7B). FIG. 7C is a graph showing the mean IL-27 signature z-scores for healthy controls and patients with SLE. The dotted line indicates where a reasonable cutoff for expression in healthy controls (the mean plus 2× the standard deviation of the healthy controls).

FIG. 8 shows that IL-27 induces IL-21 expression in T cells in vitro. FACS purified CD4⁺CD25⁻ T cells isolated from either IL-27ra^{+/+} (circles) or IL-27^{-/-} (triangles) mice were stimulated with plate-bound anti-CD3 and soluble anti-CD28 under T_{H}0 polarizing conditions and in the presence (filled symbols) or absence (open symbols) of 20 ng/ml rmIL-27 for the times indicated. IL-21 mRNA was determined by real time RT-PCR and is given relative to Rpl19.

FIG. 9 shows that IL-27 regulates expression of IL-21. CD4⁺ T cells from STAT1^{+/+} (SvEv) or STAT1^{-/-} mice were stimulated with plate-bound anti-CD3 and soluble anti-CD28 under T_{H}0 polarizing conditions and in the presence (filled symbols) or absence (open symbols) of rmIL-27 for 72 h. IL-21 in the culture supernatant was measured by ELISA.

FIG. 10 shows that IL-27 is required for IL-21 expression in vivo. Groups of IL-27ra^{+/+} (filled circles) and IL-27ra^{-/-} (open squares) mice were immunized with OVA (30 μg/ml) in CFA. 4 and 8 days after immunization, CD4+ T cells were isolated from the spleens and IL-21 mRNA was determined by real time RT-PCR (relative to Rpl19). Data from individual animals is shown. Bars indicate mean of 5 animals+/−SEM. * p<0.05 (unpaired t-test). Each of these experiments has been repeated at least 3 times.

FIG. 11 shows that IL-27ra-deficient animals have reduced numbers of T_{FH} cells. Groups of IL-27ra^{+/+} and IL-27ra^{-/-} mice were immunized twice with TNP-OVA in adjuvant and 7 days after the second immunization, tissue was collected for analysis. (A) Representative flow cytometric analysis for T_{FH} marker expression in the spleen. For all plots the CD4⁺B220⁻ gate is shown. (B) The number of CXCR5⁺PD1⁺ cells in each spleen (upper panel) or pair of draining LN (lower panel) were calculated by multiplying the percentage obtained by flow cytometry by the total cell count per organ. The average of at least 6 animals per group is given and error bars indicate SEM. * p<0.05 (unpaired t-test). These data represent 4 individual experiments. (C) The ICOS mean of fluorescence (MFI) within the CD4⁺CXCR5⁺PD1⁺ gate is given for each animal. The average of at least 6 animals per group is given and error bars indicate SEM. * p<0.05 (unpaired t-test). These data are representative of 4 individual experiments.

FIG. 12 shows that IL-27ra-deficient mice have dysfunctional germinal centers. Groups of IL-27ra^{+/+} and IL-27ra^{-/-} mice were immunized twice with TNP-OVA in adjuvant and 7 days after the second immunization tissues and sera were collected for analysis. (A) Representative flow cytometric analysis for Fas and GL7 expression in the splenic B220⁺CD4⁻ cell gate. (B) The number of GL7⁺Fas⁺B220⁺CD4⁻GC B cells in the spleen of each mouse was calculated by multiplying the percentage obtained by flow cytometry by the total cell count per organ. The average of at least 6 animals per group is given and error bars indicate SEM. (C, D & E) ELISA using plates coated with 5 μg/ml BSA-TNP₂₈ (C) or BSA-TNP₂ (D) for analysis of total anti-TNP and high affinity anti-TNP antibodies, respectively, in the serum of mice immunized as above. Anti-TNP antibodies were detected with either anti-mouse Ig (C & D) or antibodies against specific mouse Ig isotypes (E). (F) Groups of Il27ra^{+/+} and Il27ra^{-/-} mice were immunized with 100 ug of TNP-Ficoll i.p. and sera collected 5 days later. Anti-TNP-IgM levels were assessed by ELISA as in (C) and detected using anti-mouse IgM antibodies. Relative anti-TNP antibody concentration is given for each mouse, bars indicate the group average where n=6-8. * p<0.05 (unpaired t-test). (G and H)C57BL6 mice were immunized with TNP-OVA in CFA and 5 days after immunization, the indicated splenic cell populations were isolated by FACS to a purity of >99% (see methods for sort strategy). Real time RT-PCR analysis for IL-27p28 (G) and ebi3 (H) were performed and data is expressed relative to Rpl19. These data are indicative of at least 3 individual experiments.

FIG. 13 shows that IL-27 does not promote T_{FH} differentiation as a sole agent. (A & B) DO11.10tg.rag2^{-/-} or DO11.10tg.rag2^{-/-}. Il27ra^{-/-} splenocytes were activated with various concentrations of OVA₃₂₃₋₃₃₉ in the presence or absence of rmIL27 (20 ng/ml) or anti-IL-27 (10 ug/ml) for 72 hours. (A) the percentage of PD1⁺ CXCR5⁺ cells in the CD4⁺ gate (B) the percentage of AnV-neg and 7AAD-neg (viable) cells in the CD4⁺ gate. (C) CD4⁺ cells from C57BL6 mice were stimulated with plate-bound anti-CD3 and soluble anti-CD28 for 72 hours in the presence (empty histogram) or absence (shaded histogram) of rmIL-27. ICOS levels were assessed by flow cytometry. (D) Bcl6 mRNA expression levels relative to Rpl19 in OTII TCR Tg CD4⁺ T cells stimulated with OVA₃₂₃₋₃₃₉ in the presence or absence of rmIL-27 for the times indicated. (E) Thy1.1⁺OTII TCR Tg CD4⁺ T cells were isolated by magnetic purification and cultured with irradiated splenic APC plus OVA₃₂₃₋₃₃₉ peptide under TH0 conditions alone (blocking antibodies against IFNγ and IL-4 and TGFBRII-Fc), or with the addition of rmIL-21 (50 ng/ml) or rmIL-27 (50 ng/ml) for 5 days. Cells were then adoptively transferred to naïve Thy1.2 congenic hosts (n=4-8 per group) before recipient mice were subcutaneously immunized with 100 ug OVA in IFA. Two additional control groups were included which did not receive cell transfers, one group was immunized as described while the other group remained unimmunized. Seven days after immunization, differentiation of GC B cells in the LN were assessed by flow cytometry. The graph shows the average percentage of GL7⁺Fas⁺B220⁺ cells in the DLN, error bars indicate SEM.

FIG. 14 shows that IL-27 does not promote CXCR5 and PD1 expression. DO11.10tg.rag2^{-/-} splenocytes were activated with 0.03 μM OVA₃₂₃₋₃₃₉ in the presence or absence of rmIL27 for 72 hours. CXCR5 and PD1 expression in the CD4⁺ gate in the absence (filled histograms) or presence (black line) of rmIL-27.

FIG. 15 shows that IL-27 signaling to both T and B cells contributes to GC function. WT (CD45.1): IL-27ra^{-/-} (CD45.2) bone marrow chimeric mice were immunized twice with TNP-OVA as described and tissue collected assessed 7 days after the second injection. (A) The ratio of CD45.1:CD45.2 cells is given for total CD4+ cells (filled circles) and CD4⁺CXCR5⁺PD1⁺ cells (open squares) for each of 10 chimeric animals. The grey line indicates equivalency of WT and Il27ra^{-/-} cells (i.e. Ratio of 1). Bars indicate the mean±SEM. * p<0.05 (unpaired t-test). (B) The ratio of CD45.1:CD45.2 cells is given for total B220⁺ cells (filled circles) and B220⁺GL7⁺Fas⁺IgD^{lo} cells (open squares) for each of 10 chimeric animals. Bars indicate the mean±SEM.

FIG. 16 shows that the survival effect of IL-27 is IL-21 independent. The reconstitution of WT and IL-27ra−/− cell in a mixed BM chimera is similar, and mice reconstituted with IL-27ra−/− cells have reduced antigen specific IgG1 production. (A & B) TCM mice (CD45.1, Thy1.1) were lethally irradiated and reconstituted with a 50:50% mix of BM from WT (CD45.1) and Il27ra^{-/-} (CD45.2) mice. 6 weeks after BM transfer, the mice were bled to assess reconstitution by flow cytometry. (A) The percentage of WT (filled circles), Il27ra^{-/-} (open squares) and host (filled triangles) CD4⁺ T cells (B) the percentage of WT (CD45.1⁺ host plus donor; filled circles) and Il27ra^{-/-} (open squares) in the B220⁺B cell gate. (C) High affinity IgG1 levels in recipient mice reconstituted either with a mixture of bone marrows (filled triangles), IL-27Ra−/− marrow (open squares), or WT marrow (filled circles).

FIG. 17 shows that B cell-specific deletion of IL-27ra affects antibody production but not T_{FH} number. BM chimeric mice reconstituted using μMT+IL-27ra^{+/+} bone marrow or μMT+IL-27ra^{-/-} bone marrow were immunized twice as described and tissue collected assessed 7 days after the second injection. (A) The proportion of CXCR5+PD1+ cells in the CD4+B220− gate of μMT+IL-27ra^{+/+} chimeras (filled circles) or μMT+IL-27ra^{-/-} chimeras (open squares). (B) anti-TNP antibodies of the isotypes as indicated were detected by ELISA after coating with TNP₂-BSA in order to detect high affinity antibody. (C) anti-TNP antibodies of the isotypes as indicated were detected by ELISA after coating with TNP₃₀-BSA in order to detect total anti-TNP antibody. * p<0.05 (unpaired t-test). Bars indicate average+/−SEM.

FIG. 18 shows the regulation of IL-21 by IL-27. (A) CD4⁺ T cells from C57BL6 mice were stimulated with plate-bound anti-CD3 and soluble anti-CD28 under TH0 polarizing conditions in the presence (filled symbols) or absence (open symbols) of rmIL-27 and in the presence or absence of cycloheximide for 5 hours. (B) CD4+ T cells enriched from C57BL6 splenocytes were stimulated with plate-bound anti-CD3 and soluble anti-CD28 in the presence of varying concentrations of rmIL-27, rmIL-12 or both rmIL-27 and rmIL-12 for 72 hours. IL-21 in the culture supernatant was measured by ELISA. * p<0.05 (unpaired t-test). Bars indicate average+/−SEM.

FIGS. 19A and 19B show IL-27 signature genes and expression levels. These genes and probes were further selected by comparing the RNA expression level in PBMC RNA samples from lupus patients to the level in PBMC RNA samples from healthy controls. Significantly up-regulated genes (at adjusted p-value<0.001) were selected as IL-27 signature genes and probes.

### DETAILED DESCRIPTION OF THE INVENTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N. Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

“Lupus” as used herein is an autoimmune disease or disorder involving antibodies that attack connective tissue. The principal form of lupus is a systemic one, systemic lupus erythematosus (SLE), including cutaneous SLE and subacute cutaneous SLE, as well as other types of lupus (including nephritis, extrarenal, cerebritis, pediatric, non-renal, discoid, and alopecia).

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with an autoimmune disorder (e.g., lupus) are mitigated or eliminated.

As used herein, the term “prevention” includes providing prophylaxis with respect to occurrence or recurrence of a disease in an individual. An individual may be predisposed to or at risk of developing the disease but has not yet been diagnosed with the disease.

As used herein, an individual “at risk” of developing lupus may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of lupus, as known in the art. An individual having one or more of these risk factors has a higher probability of developing lupus than an individual without one or more of these risk factors.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum concentration required to effect a measurable improvement of a particular disorder (e.g., lupus). A therapeutically effective amount herein may vary according to factors such as the disease state, age, sex, and weight of the patient, and the ability of the IL-27 antagonist to elicit a desired response in the individual. A therapeutically effective amount is also one in which any toxic or detrimental effects of the IL-27 antagonist are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in subjects prior to or at an earlier stage of disease, a prophylactically effective amount may be less than a therapeutically effective amount.

“Chronic” administration refers to administration of the medicament(s) in a continuous as opposed to acute mode, so as to maintain the initial therapeutic effect (activity) for an extended period of time. “Intermittent” administration refers to treatment that is not consecutively done without interruption, but rather is cyclic in nature.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

An “individual” for purposes of treatment or prevention refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Preferably, the individual is human.

As used herein, the term “cytokine” refers generically to proteins released by one cell population that act on another cell as intercellular mediators. Examples of such cytokines include lymphokines, monokines; interleukins (“ILs”) such as IL-1, IL-1α, IL-2, IL-3, IL-4, IL-5, IL-6, IL-7, IL-8, IL-9, IL-11, IL-12, IL-13, IL-15, IL-17A-F, IL-18 to IL-29, IL-31, including PROLEUKIN® rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the term “IL-27” encompasses native sequence IL-27 heterodimer, native sequence IL-27 components Ebi3 and p28, naturally occurring variants of IL-27 heterodimer, and naturally occurring variants of IL-27 components Ebi3 and p28. IL-27 heterodimer and components thereof may be isolated from a variety of sources, such as from mammalian (including human) tissue types or from another source, or prepared by recombinant and/or synthetic methods.

As used herein, the term “IL-27 receptor” encompasses native sequence IL-27 receptor heterodimer, native sequence IL-27 receptor components IL-27Ra (also known as “WSX-1” or “TCCR”) and gp130, naturally occurring variants of IL-27 receptor heterodimer, and naturally occurring variants of IL-27 receptor components IL-27Ra and gp130. IL-27 receptor heterodimer and components thereof may be isolated from a variety of sources, such as from mammalian (including human) tissue types or from another source, or prepared by recombinant and/or synthetic methods.

As used herein, the term “IL-27 antagonist” refers to a molecule that blocks, inhibits, reduces (including significantly), or interferes with IL-27 (mammalian, such as human IL-27) biological activity in vitro, in situ, and/or in vivo, including downstream pathways mediated by IL-27 signaling, such as receptor binding and/or elicitation of a cellular response to IL-27. The term “antagonist” implies no specific mechanism of biological action whatsoever, and expressly includes and encompasses all possible pharmacological, physiological, and biochemical interactions with IL-27 whether direct or indirect, and whether interacting with IL-27, its receptors, or through another mechanism, and its consequences which can be achieved by a variety of different, and chemically divergent, compositions. Exemplary IL-27 antagonists include, but are not limited to, an anti-IL-27 antibody that specifically binds to IL-27 or one or both subunits of IL-27, an anti-sense molecule directed to a nucleic acid encoding a subunit of IL-27, a short interfering RNA (“siRNA”) molecule directed to a nucleic acid encoding one or both subunits of IL-27 (i.e., IL-27p28 or IL-27Ebi3) or IL-27Ra, an IL-27 inhibitory compound, an RNA or DNA aptamer that binds to IL-27, one or both subunits of IL-27, or to IL-27Ra, an IL-27 structural analog, a soluble IL-27Ra protein and fusion polypeptide thereof, and an anti-IL-27Ra antibody. In some embodiments, an IL-27 antagonist (e.g., an antibody) binds (physically interacts with) IL-27, binds to an IL-27Ra, reduces (impedes and/or blocks) downstream IL-27Ra signaling, and/or inhibits (reduces) IL-27 synthesis, production or release. In other embodiments, an IL-27 antagonist binds IL-27 and prevents its binding to its receptor. In still other embodiments, an IL-27 antagonist reduces or eliminates expression (i.e., transcription or translation) of IL-27, an IL-27 subunit, or IL-27Ra. Examples of types of IL-27 antagonists are provided herein.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Ten and Tristram G. Parslow (eds.), Appleton & Lange, Norwalk, Conn., 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1 and IgA2. The subunit structures and three dimensional configurations of different classes of immunoglobulins are well known and described generally in, for example, Abbas et al., Cellular and Molecular Immunology, 4^{th} ed. (W. B. Saunders Co., 2000).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” antibody is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N. Y., 1981)), recombinant DNA methods (see, e.g., U. S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U. S. Pat. Nos. 5,545,807; 5,545,806; 5,569,825; 5,625,126; 5,633,425; and 5,661,016; Marks et al., BiolTechnology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U. S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies of the invention comprise a portion of an intact antibody, generally including the antigen binding or variable region of the intact antibody or the F region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Nat'l Acad. Sci. USA 90:6444-48 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U. S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U. S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U. S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH(H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N. J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The phrase “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see U. S. Provisional Application No. 60/640,323, Figures for EU numbering).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup DI as in Kabat et al., supra.

A “VH subgroup III consensus framework” comprises the consensus sequence obtained from the amino acid sequences in variable heavy subgroup DI of Kabat et al., supra.

A “VL subgroup I consensus framework” comprises the consensus sequence obtained from the amino acid sequences in variable light kappa subgroup I of Kabat et al., supra.

An “amino-acid modification” at a specified position, e.g., of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Biotechnology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically binds to” or is “specific for” refers to measurable and reproducible interactions such as binding between a target and an antibody, that is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≦1 μM, ≦100 nM, ≦10 nM, ≦1 nM, or ≦0.1 nM. In certain embodiments, an antibody specifically binds to an epitope on a protein that is conserved among the protein from different species. In another embodiment, specific binding can include, but does not require exclusive binding.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen.

The term “solid phase” describes a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U. S. Pat. No. 4,275,149.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent-cell-mediated cytotoxicity (ADCC); phagocytosis; down regulation of cell surface receptors (e.g., B-cell receptors); and B-cell activation.

“Antibody-dependent-cell-mediated cytotoxicity” or “ADCC” refers to a form of cytotoxicity in which secreted Ig bound onto Fc receptors (“FcRs”) present on certain cytotoxic cells (e.g., natural killer (“NK”) cells, neutrophils and macrophages) enable these cytotoxic effector cells to bind specifically to an antigen-bearing target-cell and subsequently kill the target-cell with cytotoxins. The antibodies “arm” the cytotoxic cells and are required for killing of the target-cell by this mechanism. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U. S. Pat. No. 5,500,362, 5,821,337 or 6,737,056 may be performed. Useful effector cells for such assays include peripheral blood mononuclear cells (“PBMC”) and NK cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2, IgG3 and IgG4.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (see, e.g., M. Dadron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, “FcRn,” which is responsible for the transfer of maternal IgGs to the fetus. Guyer et al., J. Immunol. 117:587 (1976); and Kim et al., J. Immunol. 24:249 (1994). Methods of measuring binding to FcRn are known (see, e.g., Ghetie and Ward, Immunol. Today 18: (12):592-98 (1997); Ghetie et al., Nature Biotechnology 15(7):637-40 (1997); Hinton et al., J. Biol. Chem. 279(8):6213-16 (2004); WO 2004/92219 (Hinton et al.).

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

“Human effector cells” are leukocytes which express one or more FcRs and perform effector functions. Preferably, the cells express at least FcγRIII and perform ADCC effector function. Examples of human leukocytes which mediate ADCC include PBMCs, NK cells, monocytes, cytotoxic T-cells and neutrophils, with PBMCs and MNK cells being preferred. The effector cells may be isolated from a native source, e.g., blood.

“Complement dependent cytotoxicity” or “CDC” refers to the lysis of a target-cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed.

Polypeptide variants with altered Fc region amino acid sequences and increased or decreased C1q binding capability are described in U. S. Pat. No. 6,194,551B1 and WO99/51642. The contents of those patent publications are specifically incorporated herein by reference. See, also, Idusogie et al. J. Immunol. 164: 4178-4184 (2000).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (“Kd,” see below). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

In one embodiment, the “Kd” or “Kd value” according to this invention is measured by a radiolabeled antigen-binding assay (RIA) performed with the Fab version of an antibody of interest and its antigen as described by the following assay. Solution-binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al. J. Mol. Biol. 293:865-881 (1999)). To establish conditions for the assay, microtiter plates (DYNEX Technologies, Inc., Chantilly, Va.) are coated overnight with 5 μg/ml of a capturing anti-Fab antibody (Cappel Labs, Cochranville, Pa.) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620, Nalge Nunc International, Rochester, N. Y.), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% TWEEN-20™ surfactant in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, the Kd is measured by using surface-plasmon resonance assays using a BIACORE®-2000 or a BIACORE®-3000 instrument (BIAcore, Inc., Piscataway, N. J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20 (PBST) at 25° C. at a flow rate of approximately 25 μl/min. Association rates (kₒₙ) and dissociation rates (k_{off}) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic, Madison, Wis.) with a stirred cuvette.

An “on-rate,” “rate of association,” “association rate,” or “kₒₙ” according to this invention can also be determined as described above using a BIACORE®-2000 or a BIACORE®-3000 system (BIAcore, Inc., Piscataway, N. J.).

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, authored by Genentech, Inc. The source code of ALIGN-2 has been filed with user documentation in the U. S. Copyright Office, Washington D. C., 20559, where it is registered under U. S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Unless specifically stated otherwise, all % amino acid sequence identity values used herein are obtained as described in the immediately preceding paragraph using the ALIGN-2 computer program.

An “isolated” nucleic acid molecule encoding the antibodies herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single-stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

The term “epitope tagged” when used herein refers to a chimeric polypeptide comprising a polypeptide or antibody described herein fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM. The Ig fusions preferably include the substitution of a domain of a polypeptide or antibody described herein in the place of at least one variable region within an Ig molecule. In a particularly preferred embodiment, the immunoglobulin fusion includes the hinge, C_{H}2 and C_{H}3, or the hinge, C_{H}1, C_{H}2 and C_{H}3 regions of an IgG1 molecule. For the production of immunoglobulin fusions see also U. S. Pat. No. 5,428,130 issued Jun. 27, 1995. For example, useful immunoadhesins as medicaments include polypeptides that comprise a ligand binding subunit of IL-27 receptor or a receptor binding subunit of IL-27 is fused to a constant domain of an immunoglobulin sequence.

A “fusion protein” and a “fusion polypeptide” refer to a polypeptide having two portions covalently linked together, where each of the portions is a polypeptide having a different properly. The property may be a biological property, such as activity in vitro or in vivo. The property may also be simple chemical or physical property, such as binding to a target molecule, catalysis of a reaction, etc. The two portions may be linked directly by a single peptide bond or through a peptide linker will be in reading frame with each other.

As used herein, the term “RNA interference” or “RNAi” refers generally to a process in which a double-stranded RNA molecule or a short hairpin RNA molecule reducing or inhibiting the expression of a nucleic acid sequence with which the double-stranded or short hairpin RNA molecule shares substantial or total homology. The term “short interfering RNA” or “siRNA” or “RNAi agent” refers to an RNA sequence that elicits RNA interference. See Kreutzer et al., WO 00/44895; Zernicka-Goetz et al., WO 01/36646; Fire, WO 99/32619; Mello and Fire, WO 01/29058. As used herein, siRNA molecules include RNA molecules encompassing chemically modified nucleotides and non-nucleotides. The term “ddRNAi agent” refers to a DNA-directed RNAi agent that is transcribed from an exogenous vector. The terms “short hairpin RNA” or “shRNA” refer to an RNA structure having a duplex region and a loop region. In certain embodiments, ddRNAi agents are expressed initially as shRNAs.

As used herein, the term “aptamer” refers to a heterologous oligonucleotide capable of binding tightly and specifically to a desired molecular target, such as, for example, common metabolic cofactors (e.g., Coenzyme A, S-adenosyl methionine, and the like), proteins (e.g., complement protein C5, antibodies, and the like), or conserved structural elements in nucleic acid molecules (e.g., structures important for binding of transcription factors and the like). Aptamers typically comprise DNA or RNA nucleotide sequences ranging from about 10 to about 100 nucleotides in length, from about 10 to about 75 nucleotides in length, from about 10 to about 50 nucleotides in length, from about 10 to about 35 nucleotides in length, and from about 10 to about 25 nucleotides in length. Synthetic DNA or RNA oligonucleotides can be made using standard solid phase phosphoramidite methods and equipment, such as by using a 3900 High Throughput DNA Synthesizer™, available from Applied Biosystems (Foster City, Calif.). Aptamers frequently incorporate derivatives or analogs of the commonly occurring nucleotides found in DNA and RNA (e.g., A, G, C, and T/U), including backbone or linkage modifications (e.g., peptide nucleic acid (PNA) or phosphothioate linkages) to increase resistance to nucleases, binding avidity, or to otherwise alter their pharmacokinetic properties. Exemplary modifications are set forth in U. S. Pat. Nos. 6,455,308; 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; and in WIPO publications WO 00/56746 and WO 01/14398. Methods for synthesizing oligonucleotides comprising such analogs or derivatives are disclosed, for example, in the patent publications cited above, and in U. S. Pat. Nos. 6,455,308; 5,614,622; 5,739,314; 5,955,599; 5,962,674; 6,117,992; and in WO 00/75372.

A “stable” formulation is one in which the protein therein essentially retains its physical and chemical stability and integrity upon storage. Various analytical techniques for measuring protein stability are available in the art and are reviewed in Peptide and Protein Drug Delivery, pp. 247-301, Vincent Lee Ed., Marcel Dekker, Inc., New York, N. Y., Pubs. (1991) and Jones, A. Adv. Drug Delivery Rev. 10:29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. For example, the extent of aggregation during storage can be used as an indicator of protein stability. Thus, a “stable” formulation may be one wherein less than about 10% and preferably less than about 5% of the protein are present as an aggregate in the formulation. In other embodiments, any increase in aggregate formation during storage of the formulation can be determined.

A “reconstituted” formulation is one which has been prepared by dissolving a lyophilized protein or antibody formulation in a diluent such that the protein is dispersed throughout. The reconstituted formulation is suitable for administration (e.g. parenteral administration) to a patient to be treated with the protein of interest and, in certain embodiments of the invention, may be one which is suitable for subcutaneous administration.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example. The formulations of the present invention are hypertonic as a result of the addition of salt and/or buffer.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

A “pharmaceutically acceptable acid” includes inorganic and organic acids which are non toxic at the concentration and manner in which they are formulated. For example, suitable inorganic acids include hydrochloric, perchloric, hydrobromic, hydroiodic, nitric, sulfuric, sulfonic, sulfinic, sulfanilic, phosphoric, carbonic, etc. Suitable organic acids include straight and branched-chain alkyl, aromatic, cyclic, cycloaliphatic, arylaliphatic, heterocyclic, saturated, unsaturated, mono, di- and tri-carboxylic, including for example, formic, acetic, 2-hydroxyacetic, trifluoroacetic, phenylacetic, trimethylacetic, t-butyl acetic, anthranilic, propanoic, 2-hydroxypropanoic, 2-oxopropanoic, propandioic, cyclopentanepropionic, cyclopentane propionic, 3-phenylpropionic, butanoic, butandioic, benzoic, 3-(4-hydroxybenzoyl)benzoic, 2-acetoxy-benzoic, ascorbic, cinnamic, lauryl sulfuric, stearic, muconic, mandelic, succinic, embonic, fumaric, malic, maleic, hydroxymaleic, malonic, lactic, citric, tartaric, glycolic, glyconic, gluconic, pyruvic, glyoxalic, oxalic, mesylic, succinic, salicylic, phthalic, palmoic, palmeic, thiocyanic, methanesulphonic, ethanesulphonic, 1,2-ethanedisulfonic, 2-hydroxyethanesulfonic, benzenesulphonic, 4-chorobenzenesulfonic, napthalene-2-sulphonic, p-toluenesulphonic, camphorsulphonic, 4-methylbicyclo[2.2.2]-oct-2-ene-1-carboxylic, glucoheptonic, 4,4′-methylenebis-3-(hydroxy-2-ene-1-carboxylic acid), hydroxynapthoic.

“Pharmaceutically-acceptable bases” include inorganic and organic bases which are non-toxic at the concentration and manner in which they are formulated. For example, suitable bases include those formed from inorganic base forming metals such as lithium, sodium, potassium, magnesium, calcium, ammonium, iron, zinc, copper, manganese, aluminum, N-methylglucamine, morpholine, piperidine and organic nontoxic bases including, primary, secondary and tertiary amines, substituted amines, cyclic amines and basic ion exchange resins, [e.g., N(R′)₄⁺ (where R′ is independently H or C₁₋₄ alkyl, e.g., ammonium, Tris)], for example, isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic non-toxic bases are isopropylamine, diethylamine, ethanolamine, trimethamine, dicyclohexylamine, choline, and caffeine.

Additional pharmaceutically acceptable acids and bases useable with the present invention include those which are derived from the amino acids, for example, histidine, glycine, phenylalanine, aspartic acid, glutamic acid, lysine and asparagine.

“Pharmaceutically acceptable” buffers and salts include those derived from both acid and base addition salts of the above indicated acids and bases. Specific buffers and/or salts include histidine, succinate and acetate.

A “pharmaceutically acceptable sugar” is a molecule which, when combined with a protein of interest, significantly prevents or reduces chemical and/or physical instability of the protein upon storage. When the formulation is intended to be lyophilized and then reconstituted, “pharmaceutically acceptable sugars” may also be known as a “lyoprotectant”. Exemplary sugars and their corresponding sugar alcohols include: an amino acid such as monosodium glutamate or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; PLURONICS®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred pharmaceutically-acceptable sugars are the non-reducing sugars trehalose or sucrose. Pharmaceutically acceptable sugars are added to the formulation in a “protecting amount” (e.g. pre-lyophilization) which means that the protein essentially retains its physical and chemical stability and integrity during storage (e.g., after reconstitution and storage).

The “diluent” of interest herein is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

A “preservative” is a compound which can be added to the formulations herein to reduce bacterial activity. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

The term “pharmaceutical formulation” refers to a preparation that is in such form as to permit the biological activity of the active ingredient to be effective, and that contains no additional components that are unacceptably toxic to a subject to which the formulation would be administered. Such formulations are sterile.

A “sterile” formulation is aseptic or free from all living microorganisms and their spores.

The term “biomarker” or “marker” as used herein refers generally to a molecule, including a gene, protein, carbohydrate structure, or glycolipid, the expression of which in or on a mammalian tissue or cell or secreted can be detected by known methods (or methods disclosed herein) and is predictive or can be used to predict (or aid prediction) for a mammalian cell's or tissue's sensitivity to, and in some embodiments, to predict (or aid prediction) an individual's responsiveness to treatment regimes (such as treatments with IL-27 antagonists).

The term “sample”, as used herein, refers to a composition that is obtained or derived from an individual of interest that contains a cellular and/or other molecular entity that is to be characterized and/or identified, for example based on physical, biochemical, chemical and/or physiological characteristics. For example, the phrase “disease sample” and variations thereof refer to any sample obtained from an individual of interest that would be expected or is known to contain the cellular and/or molecular entity that is to be characterized.

By “tissue or cell sample” is meant a collection of cells obtained from a tissue of an individual or patient. The source of the tissue or cell sample may be solid tissue as from afresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. A “reference sample”, “reference cell”, or “reference tissue”, as used herein, refers to a sample, cell or tissue obtained from a source known, or believed, not to be afflicted with the disease or condition for which a method or composition of the invention is being used to identify. In one embodiment, a reference sample, reference cell or reference tissue is obtained from a healthy part of the body of the same subject or patient in whom a disease or condition is being identified using a composition or method of the invention. In one embodiment, a reference sample, reference cell or reference tissue is obtained from a healthy part of the body of an individual who is not the subject or patient in whom a disease or condition is being identified using a composition or method of the invention.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

As used herein, method for “aiding assessment” refers to methods that assist in making a clinical determination (e.g., responsiveness of lupus to treatment with IL-27 antagonists), and may or may not be conclusive with respect to the definitive assessment.

As used herein, a “reference value” can be an absolute value; a relative value; a value that has an upper and/or lower limit; a range of values; an average value; a median value; a mean value; or a value as compared to a particular control or baseline value.

The term “array” or “microarray”, as used herein refers to an ordered arrangement of hybridizable array elements, such as polynucleotide probes (e.g., oligonucleotides) and antibodies, on a substrate. The substrate can be a solid substrate, such as a glass slide, or a semi-solid substrate, such as nitrocellulose membrane. The nucleotide sequences can be DNA, RNA, or any permutations thereof.

“Amplification,” as used herein, generally refers to the process of producing multiple copies of a desired sequence. “Multiple copies” means at least 2 copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

Expression/amount of a gene or biomarker in a first sample is at a level “greater than” the level in a second sample if the expression level/amount of the gene or biomarker in the first sample is at least about 1.2×, 1.3×, 1.4×, 1.5×, 1.75×, 2×, 3×, 4×, 5×, 6×, 7×, 8×, 9× or 10× the expression level/amount of the gene or biomarker in the second sample. Expression levels/amounts can be determined based on any suitable criterion known in the art, including but not limited to mRNA, cDNA, proteins, protein fragments and/or gene copy. Expression levels/amounts can be determined qualitatively and/or quantitatively.

A “primer” is generally a short single stranded polynucleotide, generally with a free 3′-OH group, that binds to a target potentially present in a sample of interest by hybridizing with a target sequence, and thereafter promotes polymerization of a polynucleotide complementary to the target. A “pair of primers” refer to a 5′ primer and a 3′ primer that can be used to amplify a portion of a specific target gene.

The term “3” generally refers to a region or position in a polynucleotide or oligonucleotide 3′ (downstream) from another region or position in the same polynucleotide or oligonucleotide. The term “5” generally refers to a region or position in a polynucleotide or oligonucleotide 5′ (upstream) from another region or position in the same polynucleotide or oligonucleotide.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

“Detection” includes any means of detecting, including direct and indirect detection.

The term “prediction” is used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen.

“Patient response” can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e. reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the invention described herein include “comprising,” “consisting,” and “consisting essentially of” aspects and embodiments.

### III. Modes for Carrying out the Invention

The invention provides methods for treating or preventing lupus (such as systemic lupus erythematosus) in an individual comprising administering to the individual an effective amount of an IL-27 antagonist.

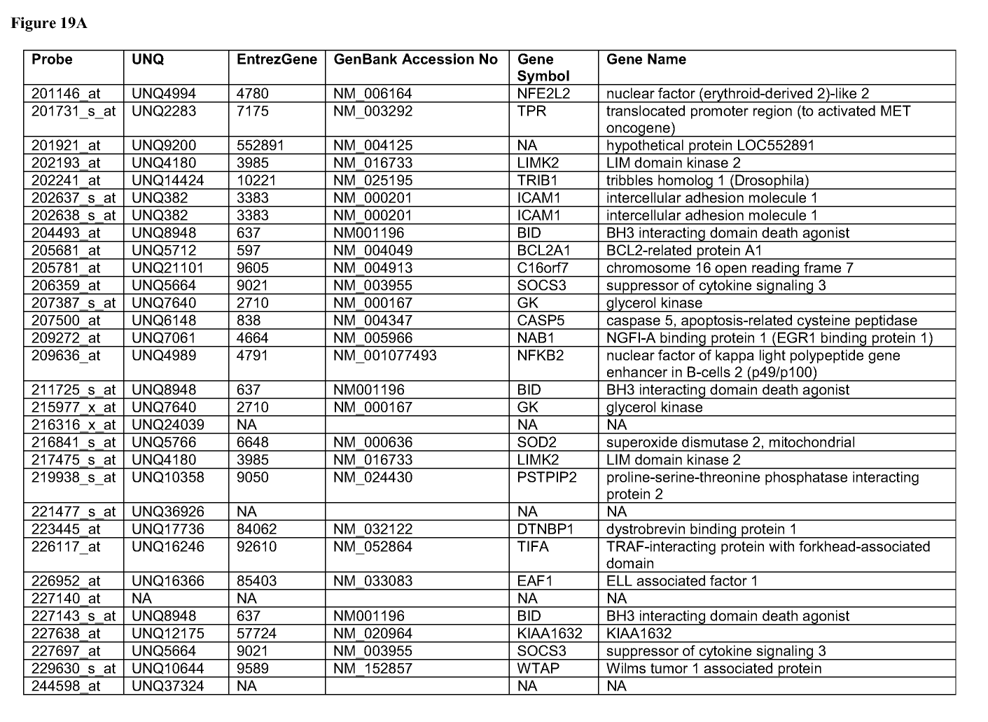

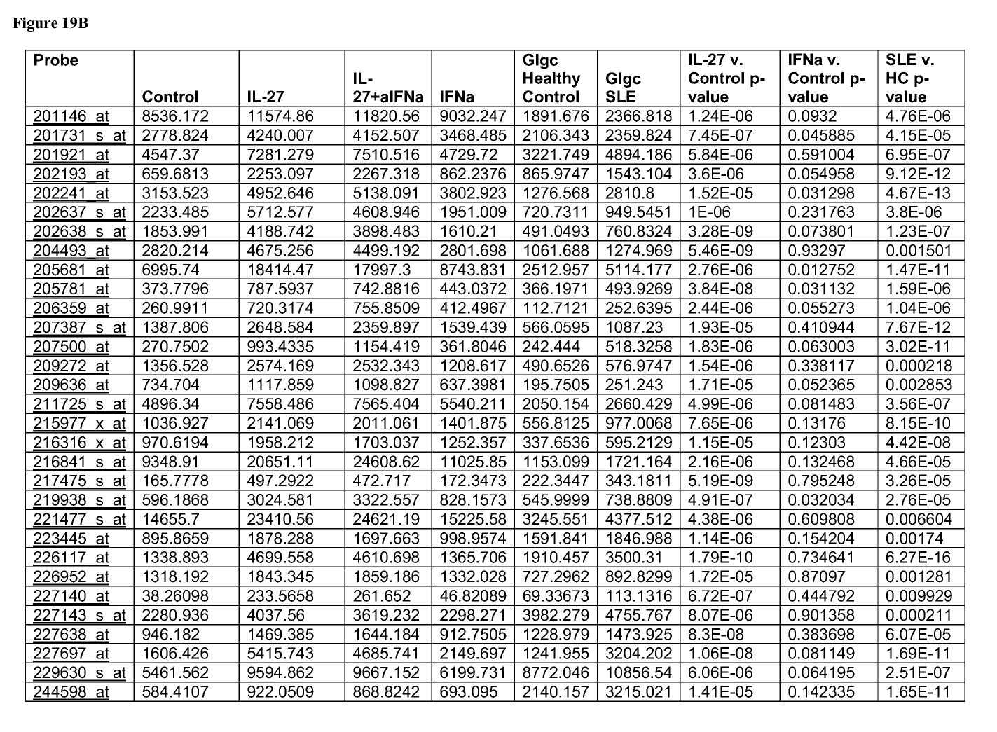

In some embodiments, the individual is selected for the IL-27 antagonist treatment based on the expression level of one or more marker genes shown in FIG. 19A in PBMCs from the individual as compared to a reference value. Methods of determining and comparing expression levels are known in the art and described herein.

With respect to all methods described herein, reference to an IL-27 antagonist also includes compositions comprising one or more of those agents. Such compositions may further comprise suitable excipients, such as pharmaceutically acceptable excipients (carriers) including buffers, acids, bases, sugars, diluents, preservatives, and the like, which are well known in the art and are described herein. The present methods can be used alone or in combination with other conventional methods of treatment.

A. IL-27 Antagonists

The methods of the invention use IL-27 antagonists, which term refers to any molecule that blocks, inhibits, reduces (including significantly), or interferes with IL-27 biological activity in vitro, in situ, and/or in vivo, including downstream pathways mediated by IL-27 signaling, such as receptor binding and/or elicitation of a cellular response to IL-27. An IL-27 antagonist should exhibit one or more of the following characteristics: (1) the ability to inhibit IL-27 biological activity and/or activity of downstream pathways mediated by IL-27 signaling; (2) the ability to block or reduce IL-27 receptor activation; (3) the ability to increase clearance of IL-27; (4) the ability to inhibit or reduce IL-27 synthesis, production or release; (5) the ability to reduce the number of T_{FH} cells; (6) the ability to reduce IL-21 expression (such as at mRNA level and/or at protein level) in T_{FH} cells; (7) the ability to reduce the amount of high affinity antibodies; and (8) the ability to treat, ameliorate, or prevent any aspect of lupus (such as SLE).

Exemplary IL-27 antagonists include, but are not limited to, anti-IL-27 antibodies that specifically bind to a subunit of IL-27 (IL-27p28 or IL-27Ebi3), or heterodimeric IL-27, anti-IL-27 receptor antibodies that specifically bind to a component of IL-27 receptor (such as IL-27Ra) or the heterodimeric IL-27 receptor, antisense molecules directed to a subunit of IL-27 (i.e., IL-27 p28 or IL-27Ebi3) or IL-27Ra, a short interfering RNA (“siRNA”) molecule directed to a nucleic acid a subunit of IL-27 (i.e., IL-27p28 or IL-27Ebi3) or IL-27Ra, an IL-27 inhibitory compound, an RNA or DNA aptamer that binds to IL-27, IL-27p28, IL-27 Ebi3, the heterodimeric IL-27 receptor, or IL-27Ra, an IL-27 structural analog, an IL-27Ra structural analog, a soluble receptor IL-27Ra and fusion polypeptide thereof, a subunit of IL-27 that binds to IL-27 receptor and a fusion polypeptide thereof, an IL-27 binding polypeptide, compounds that specifically inhibit IL-27 synthesis and/or release, and compounds that specifically inhibit IL-27Ra signal transduction.

In certain embodiments, the IL-27 antagonist inhibits IL-27 signal transduction. In certain embodiments, the IL-27 antagonist inhibits the production of IL-10 (for example, IL-27-induced IL-10 production). In certain embodiments, the IL-27 antagonist inhibits the production of IL-21 (for example, IL-27-induced IL-21 production). In certain embodiments, the IL-27 antagonist reduces the number of Tⱼ cells. In certain embodiments, the IL-27 antagonist reduces the amount of high affinity antibodies.

In certain embodiments, the IL-27 antagonist is an antibody that binds or physically interacts with a subunit of IL-27 (IL-27p28 or IL-27Ebi3). In certain embodiments, the antibody binds to IL-27p28 or IL-27Ebi3 and blocks and/or prevents formation of the heterodimeric IL-27. In certain embodiments, the antibody binds to IL-27p28 and blocks and/or prevents formation of the heterodimeric IL-27. In certain embodiments, the antibody binds to IL-27Ebi3 and blocks and/or prevents formation of the heterodimeric IL-27. In certain embodiments, the antibody is an anti-IL-27p28 antibody.

In certain embodiments, the IL-27 antagonist is an antibody that binds or physically interacts with heterodimeric IL-27, and blocks interactions between IL-27 and its receptor. In certain embodiments, the antibody binds to an epitope on the p28 subunit of IL-27. In certain embodiments, the antibody binds to an epitope on the Ebi3 subunit of IL-27. In certain embodiments, the antibody binds to both subunits of IL-27.

In certain embodiments, the IL-27 antagonist is an antibody that binds or physically interacts with IL-27Ra. In certain embodiments, the antibody binds IL-27Ra and inhibits and/or prevents formation of heterodimeric IL-27 receptor. In certain embodiments, the antibody binds IL-27Ra and inhibits and/or prevents binding between IL-27 and IL-27Ra.

In certain embodiments, the IL-27 antagonist is an antibody that binds or physically interacts with the heterodimeric IL-27 receptor, and reduces, impedes, or blocks downstream IL-27 signaling.

The antibody may have nanomolar or even picomolar affinities for the target antigen (e.g., IL-27, IL-27p28, IL-27Ebi3, IL-27 receptor, or IL-27Ra). In certain embodiments, the Kd of the antibody is about 0.05 to about 100 nM. For example, Kd of the antibody is any of about 100 nM, about 50 nM, about 10 nM, about 1 nM, about 500 pM, about 100 pM, or about 50 pM to any of about 2 pM, about 5 pM, about 10 pM, about 15 pM, about 20 pM, or about 40 pM.

In certain embodiments, the IL-27 antagonist is a small molecule antagonist, including, but is not limited to, small peptides or peptide-like molecules, soluble peptides, and synthetic non-peptidyl organic or inorganic compounds. A small molecule antagonist may have a molecular weight of any of about 100 to about 20,000 daltons (Da), about 500 to about 15,000 Da, about 1000 to about 10,000 Da. In certain embodiments, an IL-27 antagonist comprises a small molecule that binds IL-27. Exemplary sites of small molecule binding include, but are not limited to, the portion of IL-27 that binds to the IL-27 receptor, to IL-27Ra or to the portions of IL-27 adjacent to the IL-27 receptor binding region and which are responsible in whole or in part for establishing and/or maintaining the correct three-dimensional conformation of the receptor binding portion of IL-27. In certain other embodiments, an IL-27 antagonist comprises a small molecule that binds to the IL-27 receptor or to IL-27Ra and inhibits an IL-27 biological activity. Exemplary sites of small molecule binding include, but are not limited to, those portions of the IL-27 receptor and/or IL-27Ra that bind to IL-27.

In certain embodiments, the IL-27 antagonist is an RNA or DNA aptamer that binds or physically interacts with IL-27, and blocks interactions between IL-27 and its receptor. In certain embodiments, the aptamer comprises at least one RNA or DNA aptamer that binds to the p28 subunit of IL-27. In certain embodiments, the aptamer comprises at least one RNA or DNA aptamer that binds to the Ebi3 subunit of IL-27. In certain embodiments, the IL-27 antagonist comprises at least one RNA or DNA aptamer that binds to both subunits of IL-27.

In certain embodiments, the IL-27 antagonist is an RNA or DNA aptamer that binds or physically interacts with the heterodimeric IL-27 receptor or the IL-27Ra subunit, and reduces, impedes, or blocks downstream IL-27 signaling.

In certain embodiments, the IL-27 antagonist comprises at least one IL-27 or IL-27 receptor structural analog. The terms IL-27 structural analogs and IL-27 receptor structural analogs refer to compounds that have a similar three dimensional structure as part of that of IL-27 or IL-27 receptor, or IL-27Ra and which bind to IL-27 (e.g., IL-27 receptor or IL-27Ra structural analogs) or to IL-27 receptor (e.g., IL-27, IL-27p28, and IL-27Ebi3 structural analogs) under physiological conditions in vitro or in vivo, wherein the binding at least partially inhibits an IL-27 biological activity or an IL-27 receptor biological activity. Suitable IL-27 structural analogs and IL-27 receptor structural analogs can be designed and synthesized through molecular modeling of IL-27 receptor binding. The IL-27 structural analogs and IL-27 receptor structural analogs can be monomers, dimers, or higher order multimers in any desired combination of the same or different structures to obtain improved affinities and biological effects.

In certain embodiments, an IL-27 antagonist comprising at least one soluble IL-27 receptor (e.g., IL-27Ra) or fusion polypeptide thereof is provided. In certain embodiments, the soluble IL-27Ra is fused to an immunoglobulin constant domain, such as an Fc domain.

In certain embodiments, the IL-27 antagonist comprises at least one antisense molecule capable of blocking or decreasing the expression of functional IL-27 or IL-27 receptor by targeting nucleic acids encoding a subunit of IL-27 (i.e., IL-27p28 or IL-27Ebi3), or IL-27Ra. Nucleotide sequences of IL-27 and IL-27 receptor are known. See, e.g., GenBank Accession Nos. NM 005755 (human IL-27Ebi3 mRNA); NM 145659 (human IL-27p28 mRNA); and NM 004843 (human IL-27Ra mRNA). Methods are known for the preparation of antisense oligonucleotide molecules that will specifically bind one or more of IL-27p28, IL-27Ebi3, and IL-27Ra mRNA without cross-reacting with other polynucleotides. Exemplary sites of targeting include, but are not limited to, the initiation codon, the 5′ regulatory regions, including promoters or enhancers, the coding sequence, including any conserved consensus regions, and the 3′ untranslated region. In certain embodiments, the antisense oligonucleotides are about 10 to about 100 nucleotides in length, about 15 to about 50 nucleotides in length, about 18 to about 25 nucleotides in length, or more. In certain embodiments, the oligonucleotides further comprise chemical modifications to increase nuclease resistance and the like, such as, for example, phosphorothioate linkages and 2′-O-sugar modifications known to those of ordinary skill in the art.

In certain embodiments, the IL-27 antagonist comprises at least one siRNA molecule capable of blocking or decreasing the expression of functional IL-27 or IL-27 receptor by targeting nucleic acids encoding IL-27, a subunit of IL-27 (i.e., IL-27p28 or IL-27Ebi3), or IL-27Ra. It is routine to prepare siRNA molecules that will specifically target one or more of IL-27p28, IL-27Ebi3, and IL-27Ra mRNA without cross-reacting with other polynucleotides.

siRNA molecules may be generated by methods known in the art such as by typical solid phase oligonucleotide synthesis, and often will incorporate chemical modifications to increase half life and/or efficacy of the siRNA agent, and/or to allow for a more robust delivery formulation. Alternatively, siRNA molecules are delivered using a vector encoding an expression cassette for intracellular transcription of siRNA.

IL-27 antagonists can be identified or characterized using methods known in the art, such as protein-protein binding assays, biochemical screening assays, immunoassays, and cell-based assays, which are well known in the art.

To identify a molecule that inhibits interaction between IL-27 and its receptor, binding assays may be used. For example, IL-27 or receptor polypeptide is immobilized on a microtiter plate by covalent or non-covalent attachment. The assay is performed by adding the non-immobilized component (ligand or receptor polypeptide), which may be labeled by a detectable label, to the immobilized component, in the presence or absence of the testing molecule. When the reaction is complete, the non-reacted components are removed and binding complexes are detected. If formation of binding complexes is inhibited by the presence of the testing molecule, the testing molecule may be a candidate antagonist that inhibits binding between IL-27 and its receptor.

A cell-based assay may also be used to identify IL-27 antagonists. For example, IL-27 may be added to a cell along with the testing molecule to be screened for a particular activity (e.g., expression of IL-10 or IL-21), and the ability of the testing molecule to inhibit the activity of interest indicates that the testing molecule is an IL-27 antagonist.

By detecting and/or measuring levels of IL-27 gene expression, antagonist molecules that inhibit IL-27 gene expression may be tested. IL-27 gene expression can be detected and/or measured by a variety of methods, such as real time RT-PCR, enzyme-linked immunosorbent assay (“ELISA”), Northern blotting, or flow cytometry.

B. Recombinant Preparation of IL-27 Antagonists

The invention also provides methods of producing IL-27 polypeptide antagonists (such as antibodies) using recombinant techniques. For example, polypeptides can be prepared using isolated nucleic acids encoding such polypeptides (for example, anti-IL-27, anti-IL-27p28, anti-IL-27Ebi3, anti-IL-27 receptor and anti-IL-27Ra antibodies) or fragments thereof, vectors and host-cells comprising such nucleic acids. Although the methods described under Section B generally refer to production of antibodies, these methods may also be used to produce any polypeptides described herein.

For recombinant production of antibodies or fragments thereof, nucleic acids encoding the desired antibodies or antibody fragments are isolated and inserted into a replicable vector for further cloning (amplification of the DNA) or for expression. DNA encoding the polyclonal or monoclonal antibodies is readily isolated (e.g., with oligonucleotide probes that specifically bind to genes encoding the heavy and light chains of the antibody) and sequenced using conventional procedures. Many cloning and/or expression vectors are commercially available. Vector components generally include, but are not limited to, one or more of the following, a signal sequence, an origin of replication, one or more marker genes, a multiple cloning site containing recognition sequences for numerous restriction endonucleases, an enhancer element, a promoter, and a transcription termination sequence.

(1) Signal Sequence Component

The antibodies or fragments thereof may be produced recombinantly not only directly, but also as a fusion protein, where the antibody is fused to a heterologous polypeptide, preferably a signal sequence or other polypeptide having a specific cleavage site at the N-terminus of the mature protein or polypeptide. The heterologous signal sequence selected preferably is one that is recognized and processed (i.e., cleaved by a signal peptidase) by eukaryotic host-cells. For prokaryotic host-cells that do not recognize and process native mammalian signal sequences, the eukaryotic (i.e., mammalian) signal sequence is replaced by a prokaryotic signal sequence selected, for example, from the group consisting of leader sequences from alkaline phosphatase, penicillinase, 1 pp, or heat-stable enterotoxin II genes. For yeast secretion the native signal sequence may be substituted by, e.g., the yeast invertase leader, factor leader (including Saccharomyces and Kluyveromyces-factor leaders), or acid phosphatase leader, the C. albicans glucoamylase leader, or the signal described in WO 90/13646. In mammalian cell expression, mammalian signal sequences as well as viral secretory leaders, for example, the herpes simplex virus gD signal, are available.

The DNA for such precursor region is ligated in reading frame to the DNA encoding the antibodies or fragments thereof.

(2) Origin of Replication

Both expression and cloning vectors contain a nucleic acid sequence that enables the vector to replicate in one or more selected host-cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, vesicular stomatitis virus (“VSV”) or bovine papilloma virus (“BPV”) are useful for cloning vectors in mammalian cells. Generally, the origin of replication component is not needed for mammalian expression vectors (the SV40 origin may typically be used only because it contains the early promoter).

(3) Selection Gene Component

Expression and cloning vectors may also contain a selection gene, known as a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host-cell. Those cells that are successfully transformed with a heterologous gene produce a protein conferring drug resistance and thus survive the selection regimen. Examples of such dominant selection strategies use the drugs neomycin, mycophenolic acid and hygromycin.

Another example of suitable selectable markers for mammalian cells are those that enable the identification of cells competent to take up the antibody- or antibody fragment-encoding nucleic acids, such as dihydrofolate reductase (“DHFR”), thymidine kinase, metallothionein-I and preferably primate metallothionein genes, adenosine deaminase, ornithine decarboxylase, and the like.

For example, cells transformed with the DHFR selection gene are first identified by culturing all of the transformants in a culture medium that contains methotrexate (Mtx), a competitive antagonist of DHFR. An exemplary host-cell strain for use with wild-type DHFR is the Chinese hamster ovary (“CHO”) cell line lacking DHFR activity (e.g., ATCC CRL-9096).

Another example of suitable selectable markers for mammalian cells are those that enable the identification of cells competent to take up the antibody- or antibody fragment-encoding nucleic acids, such as dihydrofolate reductase (“DHFR”), glutamine synthetase (GS), thymidine kinase, metallothionein-I and -II, preferably primate metallothionein genes, adenosine deaminase, ornithine decarboxylase, and the like.

Alternatively, cells transformed with the GS (glutamine synthetase) gene are identified by culturing the transformants in a culture medium containing L-methionine sulfoximine (Msx), an inhibitor of GS. Under these conditions, the GS gene is amplified along with any other co-transformed nucleic acid. The GS selection/amplification system may be used in combination with the DHFR selection/amplification system described above.

Alternatively, host-cells (particularly wild-type hosts that contain endogenous DHFR) transformed or co-transformed with DNA sequences encoding antibodies (e.g., antibodies directed to IL-27, IL-27p28, IL-27Ebi3, IL-27 receptor or IL-27Ra) or fragments thereof, wild-type DHFR protein, and another selectable marker such as aminoglycoside 3′-phosphotransferase (“APH”) can be selected by cell growth in medium containing a selection agent for the appropriate selectable marker, such as an aminoglycosidic antibiotic, e.g., kanamycin, neomycin, or G418. See U. S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow medium containing tryptophan (e.g., ATCC No. 44076 or PEP4-1). Jones, Genetics, 85:12 (1977). The presence of the trp1 lesion in the yeast host-cell genome then provides an effective environment for detecting transformation by growth in the absence of tryptophan. Similarly, Leu2-deficient yeast strains (e.g., ATCC 20,622 or 38,626) can be complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

(4) Promoter Component

Expression and cloning vectors usually contain a promoter that is recognized by the host organism and is operably linked to the nucleic acid encoding the antibodies (e.g., antibodies directed to IL-27, IL-27p28, IL-27Ebi3, IL-27 receptor and IL-27Ra) or fragments thereof. Promoters suitable for use with prokaryotic hosts include the phoA promoter, lactamase and lactose promoter systems, alkaline phosphatase promoter, a tryptophan promoter system, and hybrid promoters such as the tac promoter, although other known bacterial promoters are also suitable. Promoters for use in bacterial systems also will contain a Shine-Dalgarno (S. D.) sequence operably linked to the DNA encoding the antibodies and antibody fragments.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the polyA tail to the 3′ end of the coding sequence. All of these sequences may be inserted into eukaryotic expression vectors.

Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, and glucokinase.

Inducible promoters in yeast have the additional advantage of permitting transcription controlled by growth conditions. Exemplary inducible promoters include the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Transcription of nucleic acids encoding antibodies or fragments thereof from vectors in mammalian host-cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), by heterologous mammalian promoters, e.g., the actin promoter or an immunoglobulin promoter, and by heat-shock gene promoters, provided such promoters are compatible with the desired host-cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U. S. Pat. No. 4,419,446. A modification of this system is described in U. S. Pat. No. 4,601,978. See also Reyes et al., Nature 297:598-601 (1982), regarding methods for expression of human interferon cDNA in mouse cells under the control of a thymidine kinase promoter from herpes simplex virus. Alternatively, the Rous Sarcoma Virus long terminal repeat can be used as the promoter.

(5) Enhancer Element Component

Transcription of a DNA encoding the antibodies or fragments thereof by higher eukaryotes is often increased by inserting an enhancer sequence into the vector. Many enhancer sequences are now known from mammalian genes (globin, elastase, albumin, α-fetoprotein, and insulin). Typically, however, one of ordinary skill in the art will use an enhancer from a eukaryotic virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody- or antibody-fragment encoding sequences, but is preferably located at a site 5′ of the promoter.

(6) Transcription Termination Component

Expression vectors used in eukaryotic host-cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding antibodies or fragments thereof. One useful transcription termination component is the bovine growth hormone polyadenylation region. See WO94/11026 and the expression vector disclosed therein.

(7) Selection and Transformation of Host-Cells

Suitable host-cells for cloning or expressing the DNA encoding antibodies (e.g., antibodies directed to IL-27, IL-27p28, IL-27Ebi3, IL-27 receptor and IL-27Ra) or fragments thereof in the vectors described herein include the prokaryotic, yeast, or higher eukaryotic cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr., 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are also suitable. These examples are illustrative rather than limiting.

Full length antibodies, antibody fragments, and antibody fusion proteins can be produced in bacteria, in particular when glycosylation and Fc effector function are not needed, such as when the therapeutic antibody is conjugated to a cytotoxic agent (e.g., a toxin). Full length antibodies have greater half life in circulation. Production in E. coli is faster and more cost efficient. For expression of antibody fragments and polypeptides in bacteria, see, e.g., U. S. Pat. No. 5,648,237 (Carter et. al.), U. S. Pat. No. 5,789,199 (Joly et al.), and U. S. Pat. No. 5,840,523 (Simmons et al.) which describes translation initiation region (TIR) and signal sequences for optimizing expression and secretion. After expression, antibodies or antibody fragments are isolated from the E. coli cell paste in a soluble fraction and can be purified through, e.g., a protein A or G column depending on the isotype. Final purification can be carried out by the same process used to purify antibodies or antibody fragments expressed, e.g., in CHO cells.

In addition to prokaryotes, eukaryotic microbes such as filamentous fungi or yeast are also suitable cloning or expression hosts for antibody- or antibody-fragment encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces spp., such as K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger. For a review discussing the use of yeasts and filamentous fungi for the production of therapeutic proteins, see, e.g., Gemgross, Nat. Biotech. 22: 1409-1414 (2004).

Certain fungi and yeast strains may be selected in which glycosylation pathways have been “humanized,” resulting in the production of an antibody with a partially or fully human glycosylation pattern. See, e.g., Li et al., Nat. Biotech. 24:210-215 (2006) (describing humanization of the glycosylation pathway in Pichia pastoris); and Gerngross et al., supra.

Suitable host-cells for the expression of glycosylated antibodies or antibody fragments are derived from multicellular organisms. Examples of invertebrate cells include plant and insect-cells. Numerous baculoviral strains and variants and corresponding permissive insect host-cells from hosts such as Spodoptera frugiperda (caterpillar), Aedes aegypti (mosquito), Aedes albopictus (mosquito), Drosophila melanogaster (fruitfly), and Bombyx mori (moth) have been identified. A variety of viral strains for transfection are publicly available, e.g., the L-1 variant of Autographa californica NPV and the Bm-5 strain of Bombyx mori NPV. Such viruses may be used as the virus herein according to the present invention, particularly for transfection of Spodoptera frugiperda cells.

Plant-cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host-cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Nat'l Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N. Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as NSO and Sp2/0. For a review of certain mammalian host cell lines suitable for antibody production, see, e.g., Yazaki and Wu, Methods in Molecular Biology, Vol. 248 (B. K.C. Lo, ed., Humana Press, Totowa, N. J., 2003), pp. 255-268.

Host-cells are transformed with the above-described expression or cloning vectors for antibody or antibody fragment production and cultured in conventional nutrient media modified as appropriate for inducing promoters, selecting transformants, or amplifying the genes encoding the desired sequences.

(8) Culturing the Host-Cells

The host-cells used to produce the antibodies (e.g., antibodies directed to IL-27, IL-27p28, IL-27Ebi3, IL-27 receptor and IL-27Ra) or antibody fragments described herein may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host-cells. In addition, any of the media described in Ham et al., Meth. Enz. 58:44 (1979), Barnes et al., Anal. Biochem. 102:255 (1980), U. S. Pat. No. 4,767,704; 4,657,866; 4,927,762; 4,560,655; or 5,122,469; WIPO Publication Nos. WO 90/03430; WO 87/00195; or U. S. Patent Re. 30,985 may be used as culture media for the host-cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host-cell selected for expression, and will be apparent to the ordinarily skilled artisan.

(9) Purification of Antibody

When using recombinant techniques, the antibodies (e.g., antibodies directed to IL-27, IL-27p28, IL-27Ebi3, IL-27 receptor or IL-27Ra) or antibody fragments can be produced intracellularly, in the periplasmic space, or secreted directly into the medium. If the antibodies are produced intracellularly, as a first step, the particulate debris from either host-cells or lysed fragments is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody or antibody fragment compositions prepared from such cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies or antibody fragments that are based on human 1, 2, or 4 heavy chains (Lindmark et al., J. Immunol. Meth. 62:1-13 (1983)). Protein G is recommended for all mouse isotypes and for human 3 heavy chain antibodies or antibody fragments (Guss et al., EMBO J. 5:15671575 (1986)). The matrix to which the affinity ligand is attached is most often agarose, but other matrices are available. Mechanically stable matrices such as controlled pore glass or poly(styrene-divinyl)benzene allow for faster flow rates and shorter processing times than can be achieved with agarose. Where the antibodies or antibody fragments comprise a C_{H}3 domain, the Bakerbond ABX™ resin (J. T. Baker, Phillipsburg, N. J.) is useful for purification. Other techniques for protein purification, such as fractionation on an ion-exchange column, ethanol precipitation, Reverse Phase HPLC, chromatography on silica, heparin, SEPHAROSE™, or anion or cation exchange resins (such as a polyaspartic acid column), as well as chromatofocusing, SDS-PAGE, and ammonium sulfate precipitation are also available depending on the antibody or antibody fragment to be recovered.

Following any preliminary purification step or steps, the mixture comprising the antibody or antibody fragment of interest and contaminants may be subjected to low pH hydrophobic interaction chromatography using an elution buffer at a pH between about 2.5-4.5, preferably performed at low salt concentrations (e.g., from about 0-0.25 M salt).

In general, various methodologies for preparing antibodies for use in research, testing, and clinical applications are well-established in the art, consistent with the above-described methodologies and/or as deemed appropriate by one skilled in the art for a particular antibody of interest.

C. Antibody Preparation

The antibodies useful in the present invention can encompass monoclonal antibodies, polyclonal antibodies, antibody fragments (e.g., Fab, Fab′-SH, Fv, scFv, and F(ab′)₂), chimeric antibodies, bispecific antibodies, multivalent antibodies, heteroconjugate antibodies, fusion proteins comprising an antibody portion, humanized antibodies, and any other modified configuration of the immunoglobulin molecule that comprises an antigen recognition site of the required specificity (e.g., for IL-27, IL-27p28, IL-27Ebi3, IL-27 receptor, or IL-27Ra), including glycosylation variants of antibodies, amino acid sequence variants of antibodies, and covalently modified antibodies. The antibodies may be murine, rat, human, or of any other origin (including chimeric or humanized antibodies).

(1) Polyclonal Antibodies

Polyclonal antibodies are generally raised in animals by multiple subcutaneous (sc) or intraperitoneal (ip) injections of the relevant antigen and an adjuvant. It may be useful to conjugate the relevant antigen (e.g., purified or recombinant IL-27, IL-27p28, IL-27Ebi3, IL-27 receptor, or IL-27Ra) to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C=NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the desired antigen, immunogenic conjugates, or derivatives by combining, e.g., 100 μg (for rabbits) or 5 μg (for mice) of the protein or conjugate with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant-cell culture as protein fusions. Also, aggregating agents such as alum are suitable to enhance the immune response.

(2) Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U. S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization (e.g., purified or recombinant IL-27, IL-27p28, IL-27Ebi3, IL-27 receptor, or IL-27Ra). Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The immunizing agent will typically include the antigenic protein (e.g., purified or recombinant IL-27, IL-27p28, IL-27Ebi3, IL-27 receptor, or IL-27Ra) or a fusion variant thereof. Generally peripheral blood lymphocytes (“PBLs”) are used if cells of human origin are desired, while spleen or lymph node cells are used if non-human mammalian sources are desired. The lymphoctyes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine or human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient-cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors (available from the Salk Institute Cell Distribution Center, San Diego, Calif. USA), as well as SP-2 cells and derivatives thereof (e.g., X63-Ag8-653) (available from the American Type Culture Collection, Manassas, Va. USA). Human myeloma and mouse-human heteromyeloma cell lines have also been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen (e.g., IL-27, IL-27p28, IL-27Ebi3, IL-27 receptor, or IL-27Ra). Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen (e.g., IL-27, IL-27p28, IL-27Ebi3, IL-27 receptor, or IL-27Ra). Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose chromatography, hydroxylapatite chromatography, gel electrophoresis, dialysis, affinity chromatography, and other methods as described above.

Monoclonal antibodies may also be made by recombinant DNA methods, such as those disclosed in U. S. Pat. No. 4,816,567, and as described above. DNA encoding the monoclonal antibodies is readily isolated and sequenced using conventional procedures (e.g., by using oligonucleotide probes that specifically bind to genes encoding the heavy and light chains of murine antibodies). The hybridoma cells serve as a preferred source of such DNA. Once isolated, the DNA may be placed into expression vectors, which are then transfected into host-cells such as E. coli cells, simian COS cells, Chinese hamster ovary (CHO) cells, or myeloma cells that do not otherwise produce immunoglobulin protein, in order to synthesize monoclonal antibodies in such recombinant host-cells. Review articles on recombinant expression in bacteria of DNA encoding the antibody include Skerra et al., Curr. Opin. Immunol., 5:256-262 (1993) and Plückthun, Immunol. Rev. 130:151-188 (1992).

In certain embodiments, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) described the isolation of murine and human antibodies, respectively, from phage libraries. Subsequent publications describe the production of high affinity (nanomolar (“nM”) range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies of desired specificity (e.g., those that bind IL-27, IL-27p28, IL-27Ebi3, IL-27 receptor, or IL-27Ra).

The DNA encoding antibodies or fragments thereof may also be modified, for example, by substituting the coding sequence for human heavy- and light-chain constant domains in place of the homologous murine sequences (U. S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein (e.g., IL-27, IL-27p28, IL-27Ebi3, IL-27 receptor, or IL-27Ra antibodies or fragments thereof) may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

(3) Humanized Antibodies.

The antibodies (such as IL-27, IL-27p28, IL-27Ebi3, IL-27 receptor, or IL-27Ra antibodies) or antibody fragments of the invention may further comprise humanized or human antibodies. Humanized forms of non-human (e.g., murine) antibodies are chimeric immunoglobulins, immunoglobulin chains or fragments thereof (such as Fab, Fab′-SH, Fv, scFv, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally will also comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U. S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Nat'l Acad. Sci. USA 89:4285 (1992); Presta et al., J. Immunol. 151:2623 (1993).

Furthermore, it is important that antibodies be humanized with retention of high affinity for the antigen and other favorable biological properties. To achieve this goal, according to a preferred method, humanized antibodies are prepared by a process of analyzing the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen or antigens (e.g., IL-27, IL-2′7p28, IL-27Ebi3, IL-27 receptor, or IL-27Ra), is achieved. In general, the CDR residues are directly and most substantially involved in influencing antigen binding.

Various forms of the humanized antibody are contemplated. For example, the humanized antibody may be an antibody fragment, such as an Fab, which is optionally conjugated with one or more cytotoxic agent(s) in order to generate an immunoconjugate. Alternatively, the humanized antibody may be an intact antibody, such as an intact IgG1 antibody.

(4) Human Antibodies

Alternatively, human antibodies can be generated. For example, it is now possible to produce transgenic animals (e.g., mice) that are capable, upon immunization, of producing a full repertoire of human antibodies in the absence of endogenous immunoglobulin production. The homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Nat'l Acad. Sci. USA, 90:2551 (1993); Jakobovits et al., Nature, 362:255-258 (1993); Bruggermann et al., Year in Immunol., 7:33 (1993); U. S. Pat. Nos. 5,591,669 and WO 97/17852.

Alternatively, phage display technology can be used to produce human antibodies and antibody fragments in vitro, from immunoglobulin variable (V) domain gene repertoires from unimmunized donors. McCafferty et al., Nature 348:552-553 (1990); Hoogenboom and Winter, J. Mol. Biol. 227: 381 (1991). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Curr. Opin Struct. Biol. 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also U. S. Pat. Nos. 5,565,332 and 5,573,905.

The techniques of Cole et al., and Boerner et al., are also available for the preparation of human monoclonal antibodies (Cole et al., Monoclonal Antibodies and Cancer Therapy, Alan R. Liss, p. 77 (1985) and Boerner et al., J. Immunol. 147(1): 86-95 (1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that sen in humans in all respects, including gene rearrangement, assembly and antibody repertoire. This approach is described, for example, in U. S. Pat. Nos. 5,545,807; 5,545,806, 5,569,825, 5,625,126, 5,633,425, 5,661,016 and in the following scientific publications: Marks et al., Bio/Technology 10: 779-783 (1992); Lonberg et al., Nature 368: 856-859 (1994); Morrison, Nature 368: 812-13 (1994), Fishwild et al., Nature Biotechnology 14: 845-51 (1996), Neuberger, Nature Biotechnology 14: 826 (1996) and Lonberg and Huszar, Intern. Rev. Immunol. 13: 65-93 (1995).

Finally, human antibodies may also be generated in vitro by activated B-cells (see U. S. Pat. Nos. 5,567,610 and 5,229,275).

(5) Antibody Fragments

In certain circumstances there are advantages to using antibody fragments, rather than whole antibodies. Smaller fragment sizes allow for rapid clearance, and may lead to improved access to solid tumors.

Various techniques have been developed for the production of antibody fragments. Traditionally, these fragments were derived via proteolytic digestion of intact antibodies (see, e.g., Morimoto et al., J. Biochem. Biophys. Method. 24:107-117 (1992); and Brennan et al., Science 229:81 (1985)). However, these fragments can now be produced directly by recombinant host-cells, for example, using nucleic acids encoding antibodies to IL-27, IL-27p28, IL-27Ebi3, IL-27 receptor, or IL-27Ra as discussed above. Fab, Fv and scFv antibody fragments can all be expressed in and secreted from E. coli, thus allowing the straightforward production of large amounts of these fragments. Antibody fragments can also be isolated from the antibody phage libraries as discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host-cell culture. Production of Fab and F(ab′)₂ antibody fragments with increased in vivo half-lives are described in U. S. Pat. No. 5,869,046. In other embodiments, the antibody of choice is a single chain Fv fragment (scFv). See WO 93/16185; U. S. Pat. No. 5,571,894 and U. S. Pat. No. 5,587,458. The antibody fragment may also be a “linear antibody,” e.g., as described in U. S. Pat. No. 5,641,870. Such linear antibody fragments may be monospecific or bispecific.

(6) Bispecific and Polyspecific Antibodies

Bispecific antibodies (BsAbs) are antibodies that have binding specificities for at least two different epitopes, including those on the same or another protein (e.g., IL-27, IL-27p28, IL-27Ebi3, IL-27 receptor, or IL-27Ra). Alternatively, one part of a BsAb can be armed to bind to the target antigen, and another can be combined with an arm that binds to a triggering molecule on a leukocyte such as a T-cell receptor molecule (e.g., CD3), or Fc receptors for IgG (FcγR) such as FcγR1 (CD64), FcγRII (CD32) and FcγRIII (CD16), in order to focus and localize cellular defense mechanisms to the target antigen-expressing cell. Such antibodies can be derived from full length antibodies or antibody fragments (e.g., F(ab′)₂ bispecific antibodies).

Bispecific antibodies may also be used to localize cytotoxic agents to cells which express the target antigen. Such antibodies possess one arm that binds the desired antigen and another arm that binds the cytotoxic agent (e.g., saporin, anti-interferon-a, vinca alkoloid, ricin A chain, methotrexate or radioactive isotope hapten). Examples of known bispecific antibodies include anti-ErbB2/anti-FcγRIII (WO 96/16673), anti-ErbB2/anti-FcgRI (U. S. Pat. No. 5,837,234), anti-ErbB2/anti-CD3 (U. S. Pat. No. 5,821,337).

Methods for making bispecific antibodies are known in the art. Traditional production of full length bispecific antibodies is based on the coexpression of two immunoglobulin heavy-chain/light chain pairs, where the two chains have different specificities. Millstein et al., Nature, 305:537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low, Similar procedures are disclosed in WO 93/08829 and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only half of the bispecific molecules provides for an easy way of separation. This approach is disclosed in WO 94/04690. For further details of generating bispecific antibodies, see, for example, Suresh et al., Methods in Enzymology 121: 210 (1986).

According to another approach described in WO 96/27011 or U. S. Pat. No. 5,731,168, the interface between a pair of antibody molecules can be engineered to maximize the percentage of heterodimers which are recovered from recombinant-cell culture. The preferred interface comprises at least a part of the C_{H}3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chains(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175: 217-225 (1992) describes the production of fully humanized bispecific antibody F(ab′)₂ molecules. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T-cells, as well as trigger the lytic activity of human cytotoxic lymphocytes against human breast tumor targets.

Various techniques for making and isolating bivalent antibody fragments directly from recombinant-cell culture have also been described. For example, bivalent heterodimers have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Nat'l Acad. Sci. USA, 90: 6444-6448 (1993) has provided an alternative mechanism for making bispecific/bivalent antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific/bivalent antibody fragments by the use of single-chain Fv (sFv) dimers has also been reported. See Gruber et al., J. Immunol., 152:5368 (1994).

Antibodies with more than two valencies are also contemplated. For example, trispecific antibodies can be prepared. Tutt et al., J. Immunol. 147:60 (1991).

Exemplary bispecific antibodies may bind to two different epitopes on a given molecule (e.g., IL-27, IL-27p28, IL-27Ebi3, IL-27 receptor, or IL-27Ra). Alternatively, an arm targeting an IL-27 signaling component may be combined with an arm which binds to a triggering molecule on a leukocyte such as a T-cell receptor molecule (e.g., CD2, CD3, CD28 or B7), or Fc receptors for IgG (FcγR), such as FcγRI (CD64), FcγRII (CD32) and FcγRIII (CD16) so as to focus cellular defense mechanisms to the cell expressing the particular protein. Bispecific antibodies may also be used to localize cytotoxic agents to cells which express a particular protein. Such antibodies possess a protein-binding arm and an arm which binds a cytotoxic agent or a radionuclide chelator, such as EOTUBE, DPTA, DOTA or TETA. Another bispecific antibody of interest binds the protein of interest and further binds tissue factor (TF).

(7) Multivalent Antibodies

A multivalent antibody may be internalized (and/or catabolized) faster than a bivalent antibody by a cell expressing an antigen to which the antibodies bind. The antibodies (e.g., IL-27, IL-27p28, IL-27Ebi3, IL-27 receptor, or IL-27Ra antibodies) or antibody fragments of the present invention can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises an Fc region or a hinge region. In this scenario, the antibody will comprise an Fc region and three or more antigen binding sites amino-terminal to the Fc region. The preferred multivalent antibody herein comprises three to about eight, but preferably four, antigen binding sites. The multivalent antibody comprises at least one polypeptide chain (and preferably two polypeptide chains), wherein the polypeptide chain or chains comprise two or more variable domains. For instance, the polypeptide chain or chains may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise V_{H}-C_{H}1-flexible linker-V_{H}-C_{H}1-Fc region chain; or V_{H}-C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

(8) Heteroconjugate Antibodies

Heteroconjugate antibodies are also within the scope of the present invention. Heteroconjugate antibodies are composed of two covalently joined antibodies (e.g., IL-27, IL-27p28, IL-27Ebi3, IL-27 receptor, or IL-27Ra antibodies or antibody fragments). For example, one of the antibodies in the heteroconjugate can be coupled to avidin, the other to biotin. Such antibodies have, for example, been proposed to target immune system cells to unwanted cells, U. S. Pat. No. 4,676,980, and have been used to treat HIV infection. International Publication Nos. WO 91/00360, WO 92/200373 and EP 0308936. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U. S. Pat. No. 4,676,980. Heteroconjugate antibodies may be made using any convenient cross-linking methods. Suitable cross-linking agents are well known in the art, and are disclosed in U. S. Pat. No. 4,676,980, along with a number of cross-linking techniques.

(9) Effector Function Engineering

It may be desirable to modify the antibody of the invention to modify effector function and/or to increase serum half life of the antibody. For example, the Fc receptor binding site on the constant region may be modified or mutated to remove or reduce binding affinity to certain Fc receptors, such as FcγRI, FcγRII, and/or FcγRIII. In some embodiments, the effector function is impaired by removing N-glycosylation of the Fc region (e.g., in the CH 2 domain of IgG) of the antibody. In some embodiments, the effector function is impaired by modifying regions such as 233-236, 297, and/or 327-331 of human IgG as described in PCT WO 99/58572 and Armour et al., Molecular Immunology 40: 585-593 (2003); Reddy et al., J. Immunology 164:1925-1933 (2000).

To increase the serum half-life of the antibody, one may incorporate a salvage receptor binding epitope into the antibody (especially an antibody fragment) as described in U. S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

(10) Other Amino Acid Sequence Modifications

Amino acid sequence modifications of the antibodies described herein (e.g., IL-27, IL-27p28, IL-27Ebi3, IL-27 receptor, or IL-27Ra antibodies or antibody fragments) are contemplated. For example, it may be desirable to improve the binding affinity and/or other biological properties of the antibodies or antibody fragments. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics (i.e., the ability to bind or physically interact with IL-27, IL-27p28, IL-27Ebi3, IL-27 receptor, or IL-27Ra). The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the target antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino-(“N”) and/or carboxy-(“C”) terminal fusions ranging in length from one residue to polypeptides containing a hundred or more residues, as well as intrasequence insertions of single or multiple amino acid residues. Examples of terminal insertions include an antibody with an N-terminal methionyl residue, or the antibody fused to a cytotoxic polypeptide. Other insertional variants of the antibody molecule include the fusion to the N- or C-terminus of the antibody to an enzyme or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table A below under the heading of “preferred substitutions”. If such.substitutions result in a change in biological activity, then more substantial changes, denominated “exemplary substitutions” in Table A, or as further described below in reference to amino acid classes, may be introduced and the products screened.

TABLE A
Amino Acid Substitutions

| | Exemplary | Preferred |
|---|---|---|
| Original Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | Ala | ala |
| Ser (S) | Thr | thr |
| Thr (T) | Ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen (e.g., IL-27, IL-27p28, IL-27Ebi3, IL-27 receptor, or IL-27Ra). Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-IgE antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibodies (e.g., IL-27, IL-27p28, IL-27Ebi3, IL-27 receptor, or IL-27Ra antibodies) or antibody fragments.

(10) Other Antibody Modifications

The antibodies (e.g., IL-27, IL-27p28, IL-27Ebi3, IL-27 receptor, or IL-27Ra antibodies) or antibody fragments of the present invention can be further modified to contain additional nonproteinaceous moieties that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

D. Pharmaceutical Formulations

Therapeutic formulations of IL-27 antagonists are prepared for storage by mixing the active ingredient having the desired degree of purity with optional pharmaceutically acceptable carriers, excipients or stabilizers (Remington: The Science and Practice of Pharmacy, 20th Ed., (Gennaro, A. R., ed., Lippincott Williams & Wilkins, Publishers, Philadelphia, Pa. 2000). Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g., Zn-protein complexes), chelating agents such as EDTA and/or non-ionic surfactants, and the like.

When the therapeutic agent is an antibody fragment, the smallest inhibitory fragment which specifically binds to the binding domain of the target protein (e.g., IL-27, IL-27p28, IL-27, Ebi3, IL-27 receptor, or IL-27Ra) is preferred. For example, based upon the variable region sequences of an antibody, antibody fragments or even peptide molecules can be designed which retain the ability to bind the target protein sequence. Such peptides can be synthesized chemically and/or produced by recombinant DNA technology (see, e.g., Marasco et al., Proc. Nat'l Acad. Sci. USA 90: 7889-7893 (1993)).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (listed above); amino acids such as alanine, glycine, glutamine, asparagine, histidine, arginine, lysine, ornithine, leucine, 2-phenylalanine, glutamic acid, threonine, and the like; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for pharmaceutical formulations comprising IL-27 antagonists to be used for in vivo administration, they must be sterile. The formulation may be rendered sterile by filtration through sterile filtration membranes. The therapeutic compositions herein generally are placed into a container having a sterile access'port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The IL-27 antagonist formulations herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 20th Edition, supra.

Stability of the proteins and antibodies described herein may be enhanced through the use of non-toxic “water-soluble polyvalent metal salts”. Examples include Ca²⁺, Mg²⁺, Zn²⁺, Fe²⁺, Fe³⁺, Cu²⁺, Sn²⁺, Sn⁴⁺, Al²⁺ and Al³⁺. Exemplary anions that can form water soluble salts with the above polyvalent metal cations include those formed from inorganic acids and/or organic acids. Such water-soluble salts have are soluble in water (at 20° C.) to at least about 20 mg/ml, alternatively at least about 100 mg/ml, alternatively at least about 200 mg/ml.

Suitable inorganic acids that can be used to form the “water soluble polyvalent metal salts” include hydrochloric, acetic, sulfuric, nitric, thiocyanic and phosphoric acid. Suitable organic acids that can be used include aliphatic carboxylic acid and aromatic acids. Aliphatic acids within this definition may be defined as saturated or unsaturated C₂₋₉ carboxylic acids (e.g., aliphatic mono-, di- and tri-carboxylic acids). For example, exemplary monocarboxylic acids within this definition include the saturated C₂₋₉ monocarboxylic acids acetic, proprionic, butyric, valeric, caproic, enanthic, caprylic pelargonic and capryonic, and the unsaturated C₂₋₉ monocarboxylic acids acrylic, propriolic methacrylic, crotonic and isocrotonic acids. Exemplary dicarboxylic acids include the saturated C₂₋₉ dicarboxylic acids malonic, succinic, glutaric, adipic and pimelic, while unsaturated C₂₋₉ dicarboxylic acids include maleic, fumaric, citraconic and mesaconic acids. Exemplary tricarboxylic acids include the saturated C₂₋₉ tricarboxylic acids tricarballylic and 1,2,3-butanetricarboxylic acid. Additionally, the carboxylic acids of this definition may also contain one or two hydroxyl groups to form hydroxy carboxylic acids. Exemplary hydroxy carboxylic acids include glycolic, lactic, glyceric, tartronic, malic, tartaric and citric acid. Aromatic acids within this definition include benzoic and salicylic acid.

Commonly employed water soluble polyvalent metal salts which may be used to help stabilize the encapsulated polypeptides of this invention include, for example: (1) the inorganic acid metal salts of halides (e.g., zinc chloride, calcium chloride), sulfates, nitrates, phosphates and thiocyanates; (2) the aliphatic carboxylic acid metal salts (e.g., calcium acetate, zinc acetate, calcium proprionate, zinc glycolate, calcium lactate, zinc lactate and zinc tartrate); and (3) the aromatic carboxylic acid metal salts of benzoates (e.g., zinc benzoate) and salicylates.

Pharmaceutical formulations of IL-27 antagonists, such as those comprising small molecules, aptamers or polypeptides other than antibodies or antibody fragments, can be designed to immediately release an IL-27 antagonist (“immediate-release” formulations), to gradually release the IL-27 antagonist over an extended period of time (“sustained-release,” “controlled-release,” or “extended-release” formulations), or with alternative release profiles. The additional materials used to prepare a pharmaceutical formulation can vary depending on the therapeutic form of the formulation (e.g., whether the system is designed for immediate-release or sustained-, controlled-, or extended-release). In certain variations, a sustained-release formulation can further comprise an immediate-release component to quickly deliver a priming dose following drug delivery, as well as a sustained-release component. Thus, sustained-release formulations can be combined with immediate-release formulations to provide a rapid “burst” of drug into the system as well as a longer, gradual release. For example, a core sustained-release formulation may be coated with a highly soluble layer incorporating the drug. Alternatively, a sustained-release formulation and an immediate-release formulation may be included as alternate layers in a tablet or as separate granule types in a capsule. Other combinations of different types of drug formulations can be used to achieve the desired therapeutic plasma profile.

Exemplary sustained-release dosage formulations (discussed in Remington's Pharmaceutical Sciences 20th Edition, supra) can include a wide variety of drug delivery systems, including those that employ: (a) a reservoir system in which the drug is encapsulated in a polymeric membrane, permitting water to diffuse through the membrane to dissolve the drug, which then diffuses out of device; (b) a matrix system (gradient or monolithic) in which the drug is suspended in a polymeric matrix and gradually diffuses out as the matrix dissolves or disintegrates; (c) micro-encapsulation and coated granule systems in which particles of drug (or particles of drug and polymer) as small as 1 micrometer (“μm”; 10⁻⁶ m) in diameter are coated in a polymeric membrane, including embodiments in which particles coated with polymers having different release characteristics (e.g., pH-dependent or non-pH-dependent polymers, compounds with different degrees of water solubility, and the like) are delivered together in a single capsule; (d) solvent-activated systems, including (i) osmotically controlled devices (e.g., OROS®, Alza Corp., Mountain View, Calif.) in which an osmotic agent and a drug are encapsulated in a semi-permeable membrane, such that an osmotic gradient pulls water into the device, and increased pressure drives drug out of device via pores in the membrane; (ii) a hydrogel swelling system in which drug is dispersed in a polymer and/or a polymer is coated onto a particle of drug, wherein the polymer swells on contact with water (in certain embodiments, swelling can be pH-dependent, pH-independent, or dependent on other physical or chemical characteristics), allowing diffusion of drug out of the device; (iii) a microporous membrane system in which drug is encapsulated in a membrane that has a component that dissolves on contact with water (in certain embodiments, swelling can be pH-dependent, pH-independent, or dependent on other physical or chemical characteristics), producing pores in the membrane through which the drug diffuses; and (iv) a wax matrix system in which the drug and an additional soluble component are dispersed in wax, such that, when water dissolves the soluble component, diffusion of drug from the system is allowed; and (e) polymeric degradation systems, including (i) bulk degradation, in which drug is dispersed in a polymeric matrix, and degradation occurs throughout the polymeric structure in a random fashion, allowing drug release; and (ii) surface erosion, in which drug is dispersed in a polymeric matrix and delivered as the surface of the polymer erodes.

E. Methods of Treatment

The invention provides methods for treating or preventing lupus (such as SLE or lupus nephritis) in an individual comprising administering to the individual an effective amount of an IL-27 antagonist. In some embodiments, the individual is a human. In some embodiments, the individual has lupus or is at risk of developing lupus.

In some embodiments, an individual having lupus is one that is experiencing or has experienced one or more signs, symptoms, or other indicators of lupus or has been diagnosed with lupus, whether, for example, newly diagnosed, previously diagnosed with a new flare, or is chronically steroid dependent with a new flare. An individual having lupus may optionally be identified as one who has been screened for elevated levels of infiltrating CD20 cells or is screened using an assay to detect auto-antibodies, such as those noted below, wherein autoantibody production is assessed qualitatively, and preferably quantitatively. Exemplary such auto-antibodies associated with SLE are anti-nuclear Ab (ANA), anti-double-stranded DNA (dsDNA) Ab, anti-Sm Ab, anti-nuclear ribonucleoprotein Ab, anti-phospholipid Ab, anti-ribosomal P Ab, anti-Ro/SS-A Ab, anti-Ro Ab, and anti-La Ab.

Diagnosis of SLE may be according to current American College of Rheumatology (ACR) criteria. Active disease may be defined by one British Isles Lupus Activity Group's (BILAG) “A” criteria or two BILAG “B” criteria. Some signs, symptoms, or other indicators used to diagnose SLE adapted from: Tan et al. “The Revised Criteria for the Classification of SLE” Arth Rheum 25 (1982) may be malar rash such as rash over the cheeks, discoid rash, or red raised patches, photosensitivity such as reaction to sunlight, resulting in the development of or increase in skin rash, oral ulcers such as ulcers in the nose or mouth, usually painless, arthritis, such as non-erosive arthritis involving two or more peripheral joints (arthritis in which the bones around the joints do not become destroyed), serositis, pleuritis or pericarditis, renal disorder such as excessive protein in the urine (greater than 0.5 gm/day or 3+ on test sticks) and/or cellular casts (abnormal elements derived from the urine and/or white cells and/or kidney tubule cells), neurologic signs, symptoms, or other indicators, seizures (convulsions), and/or psychosis in the absence of drugs or metabolic disturbances that are known to cause such effects, and hematologic signs, symptoms, or other indicators such as hemolytic anemia or leukopenia (white bloodcount below 4,000 cells per cubic millimeter) or lymphopenia (less than 1,500 lymphocytes per cubic millimeter) or thrombocytopenia (less than 100,000 platelets per cubic millimeter). The leukopenia and lymphopenia must be detected on two or more occasions. The thrombocytopenia must be detected in the absence of drugs known to induce it. The invention is not limited to these signs, symptoms, or other indicators of lupus.

For the prevention or treatment of disease, the appropriate dosage of an active agent (i.e., an IL-27 antagonist), will depend on the type of disease to be treated, the severity and course of the disease, whether the agent is administered for preventive or therapeutic purposes, previous therapy, the patient's clinical history and response to the agent, and the discretion of the attending physician. The particular dosage regimen, i.e., dose, timing, and repetition, will depend on the particular individual and that individual's medical history as assessed by a physician. Typically the clinician will administer an IL-27 antagonist, such as an anti-IL-27 antibody, an anti-IL-27p28 antibody, an anti-IL-27Ebi3 antibody, an anti-IL-27 receptor antibody, or an anti-IL-27Ra antibody, until a dosage is reached that achieves the desired result.

Methods of the present invention are useful for treating, ameliorating or palliating the symptoms of lupus (such as SLE) in an individual, or for improving the prognosis of an individual suffering from lupus. The quality of life in individuals suffering from lupus may be improved, and the symptoms of lupus may be reduced or eliminated following treatment with IL-27 antagonists. Methods of the present invention are also useful for delaying development of or preventing lupus in an individual at risk of developing lupus.

Any IL-27 antagonists described herein₅ may be administered to the individual. In certain embodiments, the IL-27 antagonist is an anti-IL-27 antibody. In certain embodiments, the IL-27 antagonist is an anti-IL-27p28 antibody. In certain embodiments, the IL-27 antagonist is an anti-IL-27Ebi3 antibody. In certain embodiments, the IL-27 antagonist is an anti-IL-27 receptor antibody. In certain embodiments, the IL-27 antagonist is an anti-IL-27Ra antibody.

F. Combination Therapies

The methods of the invention can be combined with known methods of treatment for lupus (such as systemic lupus erythematosus), either as combined or additional treatment steps or as additional components of a therapeutic formulation. Alternatively, different IL-27 antagonists may be administered in combination (e.g., an anti-IL-27Ra antibody may be administered with an IL-27-specific aptamer, or an anti-IL-27 antibody may be administered with an siRNA directed to IL-27Ra). The type of combination therapy selected will depend on the clinical manifestations of the disease.

Lupus (such as systemic lupus erythematosus) can be treated by combination therapy comprising administration of IL-27 antagonists in conjunction with other standard therapies for lupus, such as immunosuppressive drugs (e.g., methotrexate, azathioprine, cyclophosphamide, chlorambucil, mycophenolate mofetil, cyclosporine, and the like), or with treatments for clinical symptoms of lupus, such as fever, headaches, or inflammation (e.g., non-opioid analgesics, non-steroidal anti-inflammatory drugs (NSA1Ds), corticosteroids, anti-malarial drugs and the like). Exemplary NSAIDs include, for example, aspirin, ibuprofen, naproxen, and sulindac. Exemplary corticosteroids include hydrocortisone, hydrocortisone acetate, cortisone acetate, tixocortol pivalate, prednisolone, methyprednisolone, prednisone, budesonide, desonide, fluocinonide, fluocinolone acetonide, halcinonide, betamethasone sodium phosphate, dexamethasone, dexamethasone sodium phosphate, and fluocortolone. Exemplary anti-malarial drugs include hydroxylchloroquine, chloroquine, and quinacrine.

G. Pharmaceutical Dosages

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of the polypeptides or antibodies described herein, normal dosage amounts may vary from about 10 ng/kg up to about 100 mg/kg of an individual's body weight or more per day, preferably about 1 mg/kg/day to 10 mg/kg/day, depending upon the route of administration. For repeated administrations over several days or longer, depending on the severity of the disease or disorder to be treated, the treatment is sustained until a desired suppression of symptoms is achieved.

An exemplary dosing regimen comprises administering an initial dose of IL-27 antagonist, such as an antagonist antibody, of about 2 mg/kg, followed by a weekly maintenance dose of about 1 mg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, and about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the IL-27 antagonist administered, can vary over time independently of the dose used.

Generally, a non-antibody IL-27 antagonist may be administered at a dose of about 0.1 mg/kg to about 300 mg/kg, in one to three doses per day. In certain embodiments, for an adult individual of normal weight, doses ranging from about 0.3 mg/kg to about 5.00 mg/kg may be administered. The particular dosage regimen, e.g., dose, timing, and repetition, will depend on the particular individual being treated, that individual's medical history, and the properties of the IL-27 antagonist being administered (e.g., the half-life of the antagonist, and other considerations known in the art).

Dosages for a particular IL-27 antagonist may be determined empirically in individuals who have been given one or more administrations of IL-27 antagonist. Individuals are given incremental doses of an IL-27 antagonist. To assess efficacy of an IL-27 antagonist, a clinical symptom of lupus (such as SLE) can be monitored.

Administration of an IL-27 antagonist according to the methods of the invention can be continuous or intermittent, depending, for example, on the recipient's physiological condition, whether the purpose of the administration is therapeutic or prophylactic, and other factors known to skilled practitioners. The administration of an IL-27 antagonist (e.g., an IL-27 antibody, an IL-27-p28 antibody, an IL-27Ebi3 antibody, an IL-27 receptor antibody, or an IL-27Ra antibody) may be essentially continuous over a preselected period of time or may be in a series of spaced doses, e.g., either during or after development of lupus (such as SLE).

Guidance regarding particular dosages and methods of delivery is provided in the literature; see, for example, U. S. Pat. No. 4,657,760; 5,206,344; or 5,225,212. It is within the scope of the invention that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

H. Administration of the Formulations

The formulations of the present invention (e.g., formulations of IL-27 antagonists), including, but are not limited to reconstituted formulations, are administered to an individual in need of treatment with the IL-27 antagonist, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes.

In preferred embodiments, the formulations are administered to the individual by subcutaneous (i.e. beneath the skin) administration. For such purposes, the formulation may be injected using a syringe. However, other devices for administration of the formulation are available such as injection devices (e.g. the INJECT-EASE™ and GENJECT™ devices); injector pens (such as the GENPEN™); auto-injector devices, needleless devices (e.g. MEDIJECTOR™ and BIOJECTOR™); and subcutaneous patch delivery systems.

The appropriate dosage (an “effective amount”) of the IL-27 antagonist will depend, for example, on the condition to be treated, the severity and course of the condition, whether the IL-27 antagonist is administered for preventive or therapeutic purposes, previous therapy, the patient's clinical history and response to the IL-27 antagonist, the type of IL-27 antagonist used, and the discretion of the attending physician. The IL-27 antagonist is suitably administered to the patient at one time or over a series of treatments and may be administered to the patient at any time from diagnosis onwards. The IL-27 antagonist may be administered as the sole treatment or as part of a combination therapy in conjunction with other drugs or therapies useful in treating lupus (such as systemic lupus erythematosus).

Where the IL-27 antagonist of choice is an antibody, from about 0.1 mg/kg to about 20 mg/kg is an initial candidate dosage for administration to an individual, whether, for example, by one or more separate administrations. However, other dosage regimens may be useful. The progress of this therapy is easily monitored by conventional techniques.

Uses for an IL-27 antagonist formulation include the treatment or prophylaxis of lupus, for example. Depending on the severity of the disease to be treated, a therapeutically effective amount (e.g., from about 1 mg/kg to about 15 mg/kg) of the IL-27 antagonist is administered to the individual.

### Nucleic Acid Formulations

Targeted delivery of therapeutic compositions containing an antisense polynucleotide, an siRNA or other RNAi agent, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods and Applications of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Nat'l Acad. Sci. USA (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. In certain embodiments, concentration ranges of about 500 ng to about 50 mg, about 1 μg to about 2 mg, about 5 μg to about 500 μg, and about 20 μg to about 100 μg/of DNA or more can also be used during a gene therapy protocol. The therapeutic polynucleotides and polypeptides of the present invention can be delivered using gene delivery vehicles. The gene delivery vehicle can be of viral or non-viral origin (see generally Jolly, Cancer Gene Therapy (1994) 1:51; Kimura, Human Gene Therapy (1994) 5:845; Connelly, Human Gene Therapy (1995) 1:185; and Kaplitt, Nature Genet. (1994) 6:148). Expression of such coding sequences can be induced using endogenous mammalian or heterologous promoters and/or enhancers, such as those discussed above. Expression of the coding sequence can be either constitutive or regulated.

Viral-based vectors for delivery of a desired polynucleotide and expression in a desired cell are well-known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U. S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) Venezuelan equine encephalitis virus (ATCC VR-923; ATCC VR-1250; ATCC VR-1249; ATCC VR-532), and adeno-associated virus (“AAV”) vectors (see, e.g., PCT Publication Nos. WO 94/12649; WO 93/03769; WO 93/19191; WO 94/28938; WO 95/11984; and WO 95/00655). Administration of DNA linked to killed adenovirus as described in Curiel, Hum. Gene Ther. (1992) 3:147, can also be used.

Non-viral delivery vehicles and methods can also be used, including, but not limited to, polycationic condensed DNA linked or unlinked to killed adenovirus alone (see, e.g., Curiel, 1992), ligand-linked DNA (see, e.g., Wu, J. Biol. Chem. (1989) 264:16985), eukaryotic cell delivery vehicles (see, e.g., U. S. Pat. No. 5,814,482; PCT Publication Nos. WO 95/07994; WO 96/17072; WO 95/30763; and WO 97/42338), and nucleic acid neutralization or fusion with cell membranes. Naked DNA can also be used. Exemplary methods using naked DNA are described in PCT Publication No. WO 90/11092 and U. S. Pat. No. 5,580,859. Liposomes that can be used as gene delivery vehicles are described in U. S. Pat. No. 5,422,120; PCT Publication Nos. WO 95/13796; WO 94/23697; WO 91/14445; and EP Patent No. 0 524 968. Additional approaches are described in Philip, Mol. Cell. Biol. (1994) 14:2411, and in Woffendin, Proc. Nat'l Acad. Sci. USA (1994) 92:1581.

I. Articles of Manufacture

In another aspect, an article of manufacture is provided which contains an IL-27 antagonist formulation and preferably provides instructions for its use in the methods of the invention. Thus, in certain embodiments, the article of manufacture comprises instructions for the use of an IL-27 antagonist in methods for treating or preventing lupus (such as systemic lupus erythematosus) in an individual comprising administering to the individual an effective amount of an IL-27 antagonist. In certain embodiments, the individual is a human.

The article of manufacture further comprises a container. Suitable containers include, for example, bottles, vials (e.g., dual chamber vials), syringes (such as single or dual chamber syringes) and test tubes. The container may be formed from a variety of materials such as glass or plastic. The container holds the formulation. The label, which is on or associated with the container, may indicate directions for reconstitution and/or use of the formulation. The label may further indicate that the formulation is useful or intended for subcutaneous or other modes of administration. The container holding the formulation may be a single-use vial or a multi-use vial, which allows for repeat administrations (e.g. from 2-6 administrations) of the reconstituted formulation. The article of manufacture may further comprise a second container comprising a suitable diluent (e.g., BWFI). Upon mixing the diluent and the lyophilized formulation, the final protein, polypeptide, or small molecule concentration in the reconstituted formulation will generally be at least 50 mg/ml. The article of manufacture may further include other materials desirable from a commercial, therapeutic, and user standpoint, including other buffers, diluents, filters, needles, syringes, and package inserts with instructions for use.

In another embodiment, the invention provides for an article of manufacture comprising the formulations described herein for administration in an auto-injector device. An auto-injector can be described as an injection device that upon activation, will deliver its contents without additional necessary action from the patient or administrator. They are particularly suited for self-medication of therapeutic formulations when the delivery rate must be constant and the time of delivery is greater than a few moments.

J. Methods and Kits for Identifying Patients for an IL-27 Antagonist Treatment

The invention provides marker genes that are expressed at significantly higher level in peripheral blood mononuclear cells (PBMCs) from lupus patients as compared to a reference level (such as a level in PBMCs from healthy controls). The invention also provides methods for selecting individuals with lupus for treatment with an IL-27 antagonist, for aiding in patient selection during the course of development of an IL-27 antagonist therapy, for preparing an expression profile for an individual having lupus, for assessing or aiding assessment of responsiveness of an individual having lupus to treatment with an IL-27 antagonist, and for predicting responsiveness or monitoring treatment/responsiveness to an IL-27 antagonist treatment in an individual having lupus. In some embodiments, the methods comprise measuring the expression level of one or more marker genes shown in FIG. 19A in a sample comprising PBMCs obtained from an individual having lupus; and comparing the measured expression level of one or more marker genes to a reference level for the respective marker gene. In some embodiments, an increase in the expression level of one or more marker genes as compared to the reference level is used for predicting, assessing, or aiding assessment of responsiveness of the individual to an IL-27 antagonist treatment, or for determining if the individual should be treated with an IL-27 antagonist treatment. In some embodiments, the methods may further comprise administering an effective amount of an IL-27 antagonist to the individual. In some embodiments, the methods comprise measuring the expression level of one or more marker genes shown in FIG. 19A in a sample comprising PBMCs obtained from an individual having lupus; and comparing the measured expression level of one or more marker genes in PBMC sample from the individual to a reference level for the respective marker gene, wherein an increase in the expression level of one or more marker genes as compared to the reference level indicates that the individual is likely to respond to an IL-27 antagonist treatment.

Marker Genes

The expression level of one or more of the marker genes in a PBMC sample relative a reference level may be used in the methods of the invention, such as to predict, assess or aid assessment of responsiveness of patients with lupus to treatment with an IL-27 antagonist, to identify patients with lupus for treatment with an IL-27 antagonist, and for preparing an expression profile for a patient with lupus.

The IL-27 signature genes refer to one or more of the genes, and corresponding gene products, listed in FIG. 19A. These genes were identified as described in Example 7. Expression levels of one or more of these genes are used in the methods of the invention. In some embodiments, expression levels of at least 2, 3, 4, 5, 6, 7, 8, 9, 10, 11, 12, 13, 14, 15, 16, 17, 18, 19, 20, 21 or any number up to all of the marker genes shown in FIG. 19A are measured and/or used in the methods of the invention.

Reference Levels

The measured expression level of one or more marker genes in a PBMC sample from a patient is compared to a reference level. In some embodiments, the reference level is determined based on the expression level of the corresponding marker gene in PBMC samples from one or more healthy individuals (such as individuals without lupus and/or other autoimmune diseases). A reference level can be an absolute value; a relative value; a value that has an upper and/or lower limit; a range of values; an average value; a median value; a mean value; or a value as compared to a particular control or baseline value calculated based on the expression level of the marker genes from one or more healthy individuals. In some embodiments, the same method (e.g., microarray, or qRT-PCR) is used for measuring expression levels of the marker genes in the samples and measuring expression levels of the corresponding marker genes in the reference samples.

Measuring Expression Levels

The invention provides methods to examine the expression level of one or more of these marker genes in a PBMC sample relative to a reference level. The methods and assays include those which examine expression of marker genes such as one or more of those listed in FIG. 19A. Expression levels may be measured at the mRNA level and/or the protein level. In some embodiments, the measured expression level of the marker gene is normalized. For example, expression level is normalized against a gene the expression level of which does not change (or does not change significantly) among different samples. In some embodiments, expression level of one or more housekeeping genes are used for normalization. The term “housekeeping gene” refers to a group of genes that codes for proteins whose activities are essential for the maintenance of cell function. These genes are typically similarly expressed in all cell types. Housekeeping genes include, without limitation, ribosomal protein L19 (NP_{—}000972), glyceraldehyde-3-phosphate dehydrogenase (GAPDH), Cypl, albumin, actins(e.g. β-actin), tubulins, cyclophilin, hypoxantine phosphoribosyltransferase (HRPT), ribosomal protein L32 (NP_{—}001007075), and ribosomal protein/genes 28S (e.g., Q9Y399) and 18S.

The invention provides methods for measuring levels of expression from a mammalian sample containing peripheral blood mononuclear cells (PBMCs). Methods of isolating PBMCs from patients and obtaining gene expression profiles are known in the art. See, e.g., Bouwens et al., Am. J. Clin. Nutr. 91:208-17, 2010; Sims et al., Methods Mol. Biol. 517:425-40, 2009. For example, PBMCs may be isolated from whole blood by standard Ficoll gradient centrifugation. The samples may be fresh or frozen. In some embodiments, the sample is fixed and embedded in paraffin or the like. The methods for measuring gene expression levels may be conducted in a variety of assay formats, including assays detecting mRNA expression, enzymatic assays detecting presence of enzymatic activity, and immunohistochemistry assays. For measuring mRNA expression levels, microarrays (gene array analysis), in situ hybridization, Northern analysis, and PCR analysis of mRNAs may be used. For measuring protein expression levels, immunohistochemical and/or Western analysis, quantitative blood based assays (as for example Serum ELISA) (e.g., to examine levels of protein expression), and/or biochemical enzymatic activity assays. Typical protocols for evaluating the status of genes and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis).

In some embodiments, the methods of the invention further include protocols which examine the expression of mRNAs, such as mRNAs of genes listed in FIG. 19A, in a tissue or cell sample. In some embodiments, expression of various biomarkers in a sample may be analyzed by microarray technologies, which examine or detect mRNAs. For example, using nucleic acid microarrays, test and control mRNA samples from test and control PBMC samples are reverse transcribed and labeled to generate cDNAs. The cDNAs are then hybridized to an array of nucleic acids immobilized on a solid support. The array is configured such that the sequence and position of each member of the array is known. For example, probes that identify a selection of genes shown in FIG. 19A may be arrayed on a solid support. Hybridization of a labeled cDNA with a particular array member indicates that the sample from which the cDNA was derived expresses that gene. Differential gene expression analysis of disease tissue or cells can provide valuable information. Microarray technology utilizes nucleic acid hybridization techniques and computing technology to evaluate the mRNA expression profile of thousands of genes within a single experiment. (See, e.g., WO 01/75166 published Oct. 11, 2001; see also, for example, U. S. Pat. No. 5,700,637, U. S. Pat. No. 5,445,934, and U. S. Pat. No. 5,807,522, Lockart, Nature Biotechnology, 14:1675-1680 (1996); Cheung, V. G. et al., Nature Genetics 21(Suppl):15-19 (1999) for a discussion of array fabrication). DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Currently two main types of DNA microarrays are being used: oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ). The Affymetrix GeneChip® system (e.g., GeneChip® Human Genome U133 Plus 2.0 array from Affymetrix, Inc. (catalog no. 900470)) is commercially available and may be used for measuring gene expression levels.

In some embodiments, expression of various marker genes in a sample may be assessed by hybridization assays using complementary DNA probes (such as in situ hybridization using labeled riboprobes, Northern blot and related techniques) and various nucleic acid amplification assays (such as RT-PCR using complementary primers, including primers specific for one or more genes listed in FIG. 19A, and other amplification type detection methods, such as, for example, branched DNA, SISBA, TMA and the like). In some embodiments, expression of one or more biomarkers may be assayed by RT-PCR. In some embodiments, the RT-PCR may be quantitative RT-PCR (qRT-PCR). In some embodiments, the RT-PCR is real-time RT-PCR. In some embodiments, the RT-PCR is quantitative real-time RT-PCR. RT-PCR assays such as quantitative PCR assays are well known in the art. In an illustrative embodiment of the invention, a method for detecting a mRNA in a biological sample comprises producing cDNA from the sample by reverse transcription using at least one primer; amplifying the cDNA so produced using a polynucleotide as sense and antisense primers to amplify cDNAs therein; and detecting the presence of the amplified cDNA of interest. In some embodiments, the real-time RT-PCR may be performed using TaqMan® chemistry (Applied Biosystems). In some embodiments, the real-time RT-PCR may be performed using TaqMan® chemistry (Applied Biosystems) and the ABI Prism® 7700 Sequence Detection System (Applied Biosystems). See, e.g., Overbergh, L. et al., J. Biomolecular Techniques 14(1): 33-43 (2003). Such methods can include one or more steps that allow one to determine the levels of mRNA, such as a mRNA of genes listed in FIG. 19A, in a biological sample. Based on the gene sequences, primers and probes may be designed for conducting qRT-PCR.

In some embodiments, the expression of proteins encoded by the genes listed in FIG. 19A in a PBMC sample is examined using immunohistochemistry and staining protocols. Immunohistochemical staining has been shown to be a reliable method of assessing or detecting presence of proteins in a sample. Immunohistochemistry (“IHC”) techniques utilize an antibody to probe and visualize cellular antigens in situ, generally by chromogenic or fluorescent methods.

In alternative methods, the sample may be contacted with an antibody specific for said biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U. S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g., 2-40 minutes or overnight if more convenient) and under suitable conditions (e.g., from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

In some embodiments, the methods involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

In some embodiments, expression of a selected marker in a cell sample may be examined by way of functional or activity-based assays. For instance, if the biomarker is an enzyme, one may conduct assays known in the art to determine or detect the presence of the given enzymatic activity in the tissue or cell sample.

Comparing Expression Levels, Identifying Patients for an IL-27 Antagonist Treatment, and Predicting, Assessing or Aiding Assessment of Responsiveness of Patients to an IL-27 Antagonist Treatment

The methods described herein comprise a process of comparing a measured expression level of a marker gene to a reference level. The reference level may be a measured expression level of the same marker gene in a different sample (e.g., one or more healthy controls). In some embodiments, the ratio of the measured expression level of the marker gene to the measured expression level of the reference is calculated, and the ratio may be used for assessing or aiding assessment of responsiveness of patients with lupus to an IL-27 antagonist treatment, or identifying patients for an IL-27 antagonist treatment. In some embodiments, the comparison is performed to determine the magnitude of the difference between the measured expression level of the marker gene in the sample from the individual and in the reference sample (e.g., comparing the fold or percentage difference between the expression levels of the marker gene in the sample from the individual and the reference sample). An increased expression of a marker gene in the sample from the individual with lupus as compared to the expression of the marker gene in the reference sample (such as healthy controls) suggests or indicates that the patient is likely to respond to an IL-27 antagonist treatment. See marker genes in FIG. 19A. In some embodiments, a fold of increase in the expression level of the sample from the individual can be at least about any of 1.2×, 1.3×, 1.4×, 1.5×, 1.75×, 2×, 3×, 4×, 5×, 6×, 7×, 8×, 9×, or 10× the expression level of the reference level.

In some embodiments, the reference level is a value or a range determined by expression levels of the corresponding marker gene in samples from healthy controls.

The comparison can be carried out in any convenient manner appropriate to the type of measured value and reference value for the gene markers at issue. The process of comparing may be manual or it may be automatic (such as using a computer or any other computation means to perform the comparison including an algorithm to determine if a patient is likely to response to an IL-27 antagonist treatment). In some embodiments, measured expression levels are normalized values. As will be apparent to those of skill in the art, replicate measurements may be taken for the expression levels of marker genes and/or reference genes. In some embodiments, replicate measurements are taking into account for the measured values. The replicate measurements may be taken into account by using either the mean or median of the measured values as the “measured value”. Statistical analysis known in the art may be used to verify the significance of the difference between the two values compared.

In some embodiments, expression levels of at least 2, 3, 4, 5, 6, 7, 8, 9, 10, 11, 12, 13, 14, 15, 16, 17, 18, 19, 20, 21 or any number up to all of the marker genes in FIG. 19A are measured, and a z-score for each gene across a SLE and healthy control gene expression data is calculated. The z-scores from a set of genes shown in FIG. 19A are averaged to create an aggregated gene expression statistics. A cutoff (e.g., at the mean plus two standard deviations of the normal patient value) is selected to stratify lupus patients into sub-populations with high expression or low expression of IL-27 signature genes. The information may be used for predicting, assessing, or aiding assessment of responsiveness of patients to an IL-27 antagonist treatment. A lupus patient with high expression of IL-27 signature genes may be treated by administering an effective amount of an IL-27 antagonist. Any of the IL-27 antagonist described herein may be used for the treatment

Kits

The invention also provides kits for measuring expression levels of one or more of the marker genes shown in FIG. 19A. Such kits may comprise at least one reagent specific for detecting the expression level of a marker gene described herein, and may further include instructions for carrying out a method described herein. In some embodiments, the kits may further comprise an IL-27 antagonist described herein for treating an individual with lupus.

In some embodiments, the kits comprise reagents for detecting the expression level of one, or more marker genes described herein. In some embodiments, the kits comprise reagents for detecting the expression level of at least 2, 3, 4, 5, 6, 7, 8, 9, 10, 11, 12, 13, 14, 15, 16, 17, 18, 19, 20, 21 or any number up to all of the marker genes shown in FIG. 19A. In some embodiments, the reagents comprise one or more polynucleotides capable of specifically hybridizing to one or more maker genes shown in FIG. 19A or complements of said genes. In some embodiments, the reagents comprise primers and primer pairs, which allow the specific amplification of the polynucleotides corresponding to the marker genes or of any specific parts thereof, and/or probes that selectively or specifically hybridize to nucleic acid molecules or to any part thereof. Probes may be labeled with a detectable marker, such as, for example, a radioisotope, fluorescent compound, bioluminescent compound, a chemiluminescent compound, metal chelator or enzyme. Such probes and primers can be used to detect the presence of polynucleotides, such as the polynucleotides corresponding to genes listed in FIG. 19A, in a sample and as a means for detecting a cell expressing of the polynucleotides corresponding to the marker genes. As will be understood by the skilled artisan, a great many different primers and probes may be prepared based on the sequences provided herein and used effectively to amplify, clone and/or determine the presence and/or levels of mRNAs. In some embodiments, the kits comprise at least one pair of primers and a probe specific for detecting one marker gene expression level using qRT-PCR. The invention provides a variety of compositions suitable for use in performing methods of the invention, which may be used in kits. For example, the kits may comprise surfaces, such as arrays that can be used in such methods. In some embodiments, an array comprises individual or collections of nucleic acid molecules useful for detecting expression level of the marker genes. For instance, an array may comprises a series of discretely placed individual nucleic acid oligonucleotides or sets of nucleic acid oligonucleotide combinations that are hybridizable to a sample comprising target nucleic acids. The reagents for detecting protein expression level of a marker gene may comprise an antibody that specifically binds to the protein encoded by the marker gene. The kit can further comprise a set of instructions and materials for preparing a PBMC sample and preparing nucleic acid (such as mRNA) from a sample.

The kits may further comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be an antibody or polynucleotide specific for a marker gene. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

The kit of the invention may typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

The invention will be more fully understood by reference to the following examples. They should not, however, be construed as limiting the scope of the invention. All citations throughout the disclosure are hereby expressly incorporated by reference.

### EXAMPLES

### Example 1

### Germinal Center B-Cells Produce High Levels of IL-27 mRNA and Protein

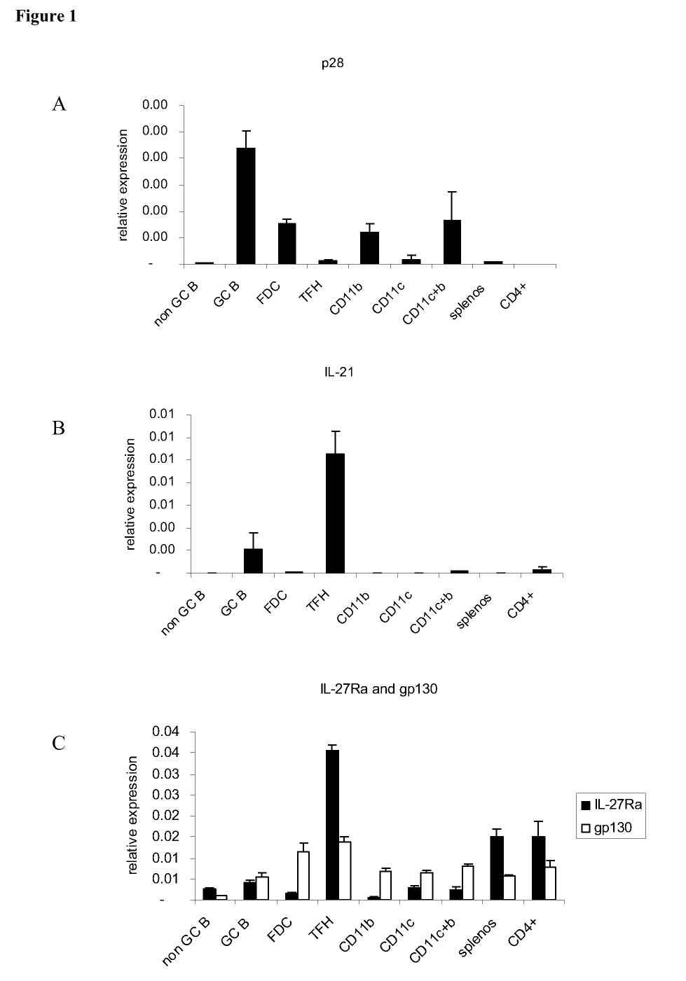

The distribution of IL-27, IL-21, IL-27Ra, and gp130 expression in various cell types present in the germinal center was assessed by quantitative RT-PCR. C57BL6 mice were immunized with 30 ug TNP-OVA in CFA and spleens were obtained 5 days later. Splenocytes were stained with various antibody cocktails and the following populations FACS sorted: non GC B cells (B220+CD38+); GC B cells (B220+CD38lo); follicular dendritic cells (FDC; CD32+FCD-M1+CD21/35+B220−); T follicular helper cells (T_{FH}; CD4+B220−CXCR5+PD1+), CD11b+ cells, CD11c+ cells, CD11b+CD11c+ cells (CD11c+b), and CD4+ cells (B220neg). Gene expression was measured by quantitative RT-PCR. The data showed that IL-21 is specifically expressed in T₁ cells, as expected (FIG. 1B). IL-27p28 is most prominently expressed in GC B-cells and FDCs, though it is also expressed in CD11b+monocytes/macrophages (FIG. 1A). The two subunits of the IL-27R (IL-27Ra and the shared subunit gp130) are expressed everywhere, although the expression is highest on T_{FH} cells, suggesting an important biological function (FIG. 1C).

IL-27 production by GC B-cells is confirmed at the level of protein expression. First, histological and flow cytometric methods for IL-27 detection using specific antibodies are optimized. Antibodies include the anti-IL-27p28 antibody mAb 4066. IL-27 staining is then used to examine specific sites of IL-27 expression within the lymphoid tissue structure after immunization. Intracellular staining and flow cytometry is performed in conjunction with the surface markers outlined in Table 1.

TABLE 1
Flow cytometric analysis of lymphocyte populations

| Cell Type | Cell surface | Intracellular |
|---|---|---|
| GC B-cells | GL7+ Fas+ IgD^{lo} CD38^{lo} B220+ | |
| T_{FH} cells | CXCR5+ ICOS+ PD1+ | IL-10+, IL-21+ |
| | CD4+B220− | |
| Viable T_{FH} cells | CXCR5+ ICOS+ PD1+ | |
| | CD4+B220−7AAD−AnV− | |
| Memory CD4 or | (CD4+ or CD8+) | |
| CD8 T-cells | CD44+CD45RB#CD62L− | |
| | CD69+/− | |
| Identification of | Thy1.1+ (CD90.1) Ab added to | |
| Thy1.1+ | above stains | |
| Proliferation | CFSE in addition to the above | |
| | stains | |
| Cytokine | CD4, CD8, B220, CD44 | IL-10, IFNγ, IL-21, |
| expression profile | | IL-4, IL-13, IL-17 |

Protein expression is also analyzed by Western blotting to show expression in FACS sorted splenic subsets. Factors involved in induction of IL-27 expression are identified by monitoring IL-27 expression by B-cells in vitro following challenge with a panel of stimuli including TLR agonists, TNF family costimulatory molecules such as BAFF and anti-CD40 crosslinking antibodies as well as BCR stimuli (anti-μ and specific antigen, HEL on SW_{HEL} BCR transgenic B-cells described in more detail below). Preliminary data shows that IL-27 expression is upregulated by CD40L stimulation suggesting that IL-27 production by B-cells is modulated by T helper cells. These experiments confirm that IL-27 protein is expressed by activated B-cells within the GC.

### Example 2

### IL-27 Induces Expression of IL-21

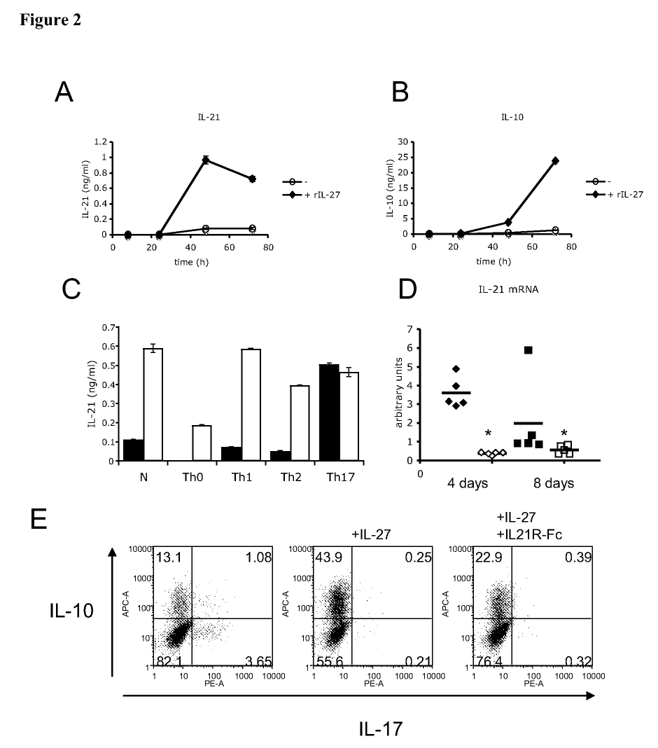

The ability of IL-27 to induce expression of IL-21 and IL-10 was confirmed at the level of mRNA and protein expression. First, purified CD4+ T-cells were stimulated with anti-CD3 and anti-CD28 in the presence of rIL-2 and antibodies blocking 1FNγ and IL-4 (T_{H}0 condition) in the presence or absence of rIL-27. The concentration of IL-21 and IL-10 in the supernatants was measured at various timepoints by ELISA (FIGS. 2A and 2B).

Next, purified CD4+ T-cells were stimulated with anti-CD3 and anti-CD28 in the presence of IL-2 and various polarization cocktails for 72 hours in the absence or presence of IL-27. In FIG. 2C, open bars represent the amount of IL-21 in the culture supernatant in the presence of IL-27; and closed bars represent the amount of IL-21 in the culture supernatant in the absence of IL-27. These experiments were repeated in the absence of IL-2, and produced the same result. The concentration of IL-21 in the culture supernatants was measured by ELISA (FIG. 2C).

Finally, IL-27Ra+/+ and IL-27Ra−/− mice were immunized with 30 μg TNP-OVA emulsified in CFA. At 4 and 8 days post-immunization, spleens were harvested and CD4+ T-cells isolated by magnetic separation. IL-21 mRNA levels were detected by RT-PCR and normalized to expression of the housekeeping gene, RPL-19 (FIG. 2D). In FIG. 2D, open symbols represent the expression of IL-21 mRNA (arbitrary units) in IL-27Ra−/− mice, and closed symbols represent the expression of IL-21 mRNA (arbitrary units) in wildtype (IL-27R1+/+) mice. Bars indicate the average value for each group. An asterisk (*) indicates a p<0.05 by the Wilcoxon test.

The experiment shown in FIG. 2E demonstrated that IL-27 induced IL-10 production was dependent on the presence of IL-21. If IL-21 signaling is blocked with a soluble IL-21R-Fc (third FACS plot), the frequency of IL-10 producing cells was diminished.

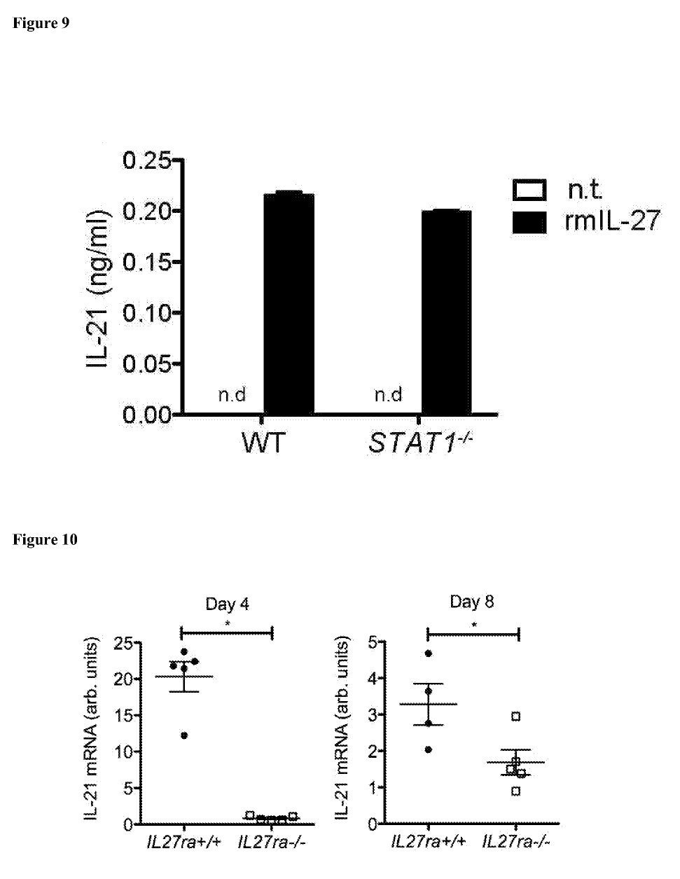

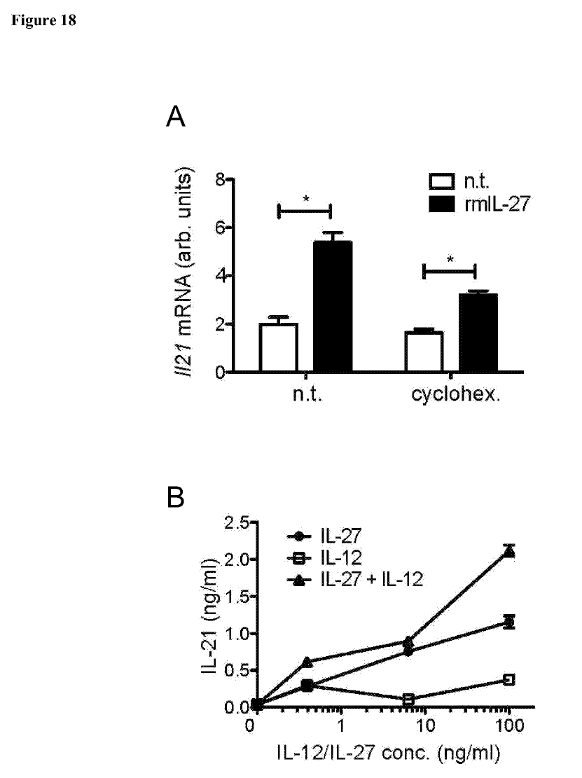

IL-27 induces IL-21 expression in vitro and in vivo. The effect of IL-27 on FACS purified naïve T cells during anti-CD3/anti-CD28 stimulation was tested. The addition of IL-27 resulted in greatly elevated IL-21 mRNA expression (FIG. 8), peaking at around 48 hours. This response was specific to rmIL-27 since no effect was observed on IL-27rd T cells (FIG. 8). IL-27 was able to enhance IL-21 mRNA expression even in the presence of the translational repressor cycloheximide, albeit to a lesser extent (FIG. 18A), suggesting that IL-27 directly enhances IL-21 expression while a feed-forward effect such as autocrine IL-21 signalling may still contribute (Nurieva et al., 2008). ELISA of the culture supernatants confirmed that IL-27 induced IL-21 protein production with similar kinetics. Furthermore, IL-27 induced IL-21 under all T helper in vitro polarizing conditions except in T_{H}17 stimulating conditions, which have previously been reported to elicit high levels of IL-21 production (FIG. 2C) (Korn et al., 2007; Nurieva et al., 2007; Wei et al., 2007; Zhou et al., 2007). Since IL-6 can induce IL-21 expression (Nurieva et al., 2007; Zhou et al., 2007), it is likely that STAT3 activation by IL-27 is unable to further enhance IL-21 expression when a strong IL-6 signal is given, as is the case in the T_{H}17 condition. Interestingly, while IL-27 suppresses T_{H}17 differentiation and IL-17 expression under these conditions (Batten et al., 2006), the IL-21 expression remained elevated, but was not further enhanced, by rmIL-27. Thus, IL-21 expression is not exclusive to the T_{H}17 phenotype but rather is subject to its own STAT3 dependent regulatory mechanism (Wei et al., 2007). A prominent feature of IL-27 signaling is activation of STAT1, although induction of IL-21 protein expression is not dependent on activation of this transcription factor (FIG. 9). IL-12 and IL-27 had an additive effect on IL-21 secretion by murine CD4⁺ T cells (FIG. 18B).

Having established that IL-27 is sufficient to induce IL-21 in vitro, it was determined whether IL-27 signaling is required for IL-21 induction in vivo. To this end, WT and IL-27Ra deficient (IL-27ra^{-/-}) mice were immunized with OVA/CFA and measured IL-21 mRNA expression in splenic CD4⁺ T cells 4 and 8 days after immunization. CD4⁺ T cells from IL-27ra^{-/-} mice contained significantly diminished levels of IL-21 mRNA, demonstrating that IL-27 signals are non-redundant for IL-21 expression in vivo (FIG. 10).

### Example 3

### IL-27 is Essential for the Formation and/or Maintenance of GC Reactions

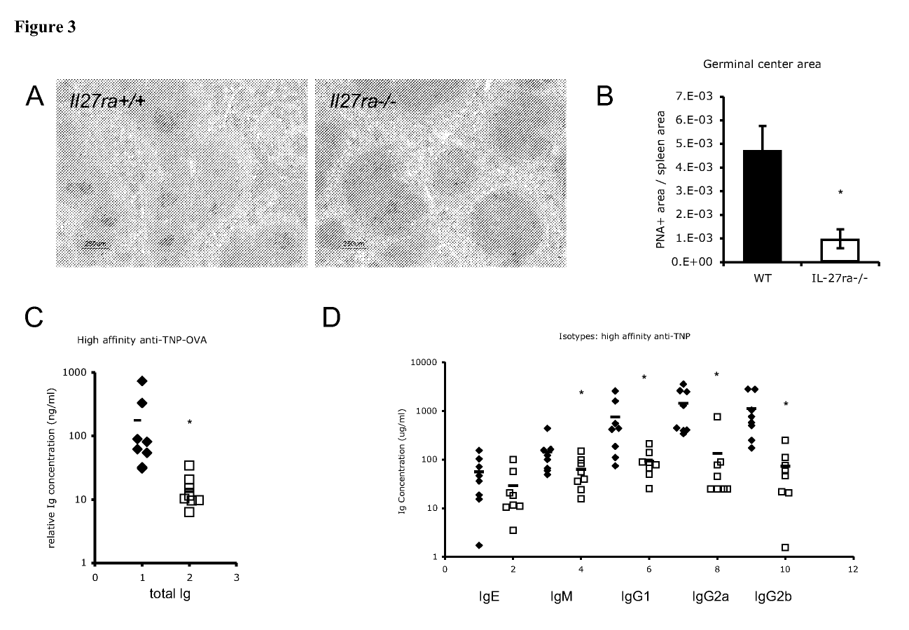

The T-dependent antigen response was examined in IL-27R^{-/-} mice. To study GC formation and function, mice were immunized with CFA+OVA followed by IFA+TNP−OVA 21 days later. Seven days after the second immunization spleens and lymph nodes were harvested, and GCs were stained with PNA and visualized histologically. The number and size of GC were reduced in IL-27Ra deficient mice (FIG. 3A) and electronic quantitation of PNA+GC area in the spleens of 8 mice per group revealed that this reduction was statistically significant (FIG. 3B). Next, BSA was used to capture high affinity antibodies, which were detected with anti-mouse IgG; IL-27R^{-/-} animals produced fewer high affinity IgG antibodies. The results are shown in FIG. 3C. The P-value is 0.00044, if the T-test is done with the log₁₀ of the Ig concentration. The analysis shown in FIG. 3C was repeated but detection was carried out with Ig isotype-specific detection antibodies, showing that all isotypes are affected (FIG. 3D). In FIGS. 3C and 3D, open symbols represent IL-27Ra−/− mice, and closed symbols represent wildtype (IL-27Ra+/+) mice.

Published data indicates that GC activity in response to TNP-OVA in CFA peaks at 6-10 days post immunization. Garside et al., Science 281:96-99 (1998). Therefore, IL-27Ra^{-/-} and IL-27Ra^{+/+} mice are immunized with 30 μg TNP-OVA in CFA. The progression of GCs is followed over several timepoints post-immunization (2, 4, 6, 8, 10 and 14 days). At those times GC activity is investigated using immunohistochemistry to visualize GC structures (e.g., PNA and FDC-M1), flow cytometry to quantitate the number of GC B and T_{FH} cells, and ELISA to detect high and low affinity anti-TNP antibodies and assess isotype switching.

Those experiments are performed as follows: at each time point after immunization, the mice are sacrificed and the blood is collected for preparation of serum. Draining inguinal lymph nodes and the spleen are also harvested. The spleen is bisected and weighed to allocate the portions for histology and flow cytometry. Spleen sections are stained using PNA to detect GC B-cells, FDC-M1 or CR1 to detect FDC networks or other specific markers relevant to the particular experiment. Spleen and lymph node tissue for flow cytometry is disrupted mechanically, red blood cells are lysed and the total cell number from each organ is counted. Cell suspensions are stained with fluorescent antibody cocktails to determine the proportion of GC B-cells and T_{FH} cells by flow cytometry (see Table 1 above) and the total number of each population is calculated from the cell counts. If intracellular staining of cytokines is to be investigated, the cell suspensions are first stimulated in vitro with PMA/ionomycin for 4 hours in the presence of brefeldin A to prevent cytokine secretion before performing intracellular staining. Antigen specific antibody production, isotype switching and the emergence of high affinity antibodies in the serum are measured by ELISA via standard procedures.

Next, BrdU incorporation experiments are performed to assess the proliferation and turnover of GC B and T_{FH} T-cells. BrdU is given intraperitoneally (0.8 mg per mouse in 200 μl of PBS) at the time of immunization and then supplied in the drinking water at 0.8 mg/ml for the following 6 days. Samples obtained at 2, 4 and 6 days are analyzed to estimate proliferation of GC B and T_{FH} cells in the early response. Samples taken at subsequent timepoints at 6, 8, 10, 14 and 21 days are analyzed to assess the survival of the responding cells. At each time point BrdU is detected by flow cytometry in combination with GC B and T_{FH} cell staining combinations indicated in Table 1.

IL-27Ra^{-/-} and IL-27Ra^{+/+} mice are re-immunized 21 days after priming to examine the secondary response. Since this is a memory response, timepoints early after re-immunization are examined (2, 4 and 6 days) using the parameters outlined above. To ensure that a reservoir of antigen and CFA does not persist at the injection site, LPS-matured antigen-loaded BM derived DC is used to immunize the mice via intravenous injection.

Multiple cytokine deficiencies have been shown to influence the GC response. Since infections and immunization strategies leading to T_{H}1-, T_{H}17- or T_{H}2-biased responses are all associated with GC formation, different cytokines may be important for GC function in different types of responses. For instance, IL-27 has been associated with the T_{H}1 response, and appears to be essential for GC activity during immunization with CFA, which contains mycobacterium. To assess the role IL-27 plays in responses than other T_{H}1, mice are immunized with TNP-OVA emulsified in CFA (T_{H}1 and T_{H}17 skewing) or alum (T_{H}2 skewing), sheep red blood cells (unknown) and antigen loaded DCs activated by exposure to LPS (T_{H}1 skewing). The GC response is assessed 7 days post-immunization in IL-27Ra^{-/-} and ^{+/+} mice using flow cytometry and histology and ELISA assessment of serum antibody isotype as described above.

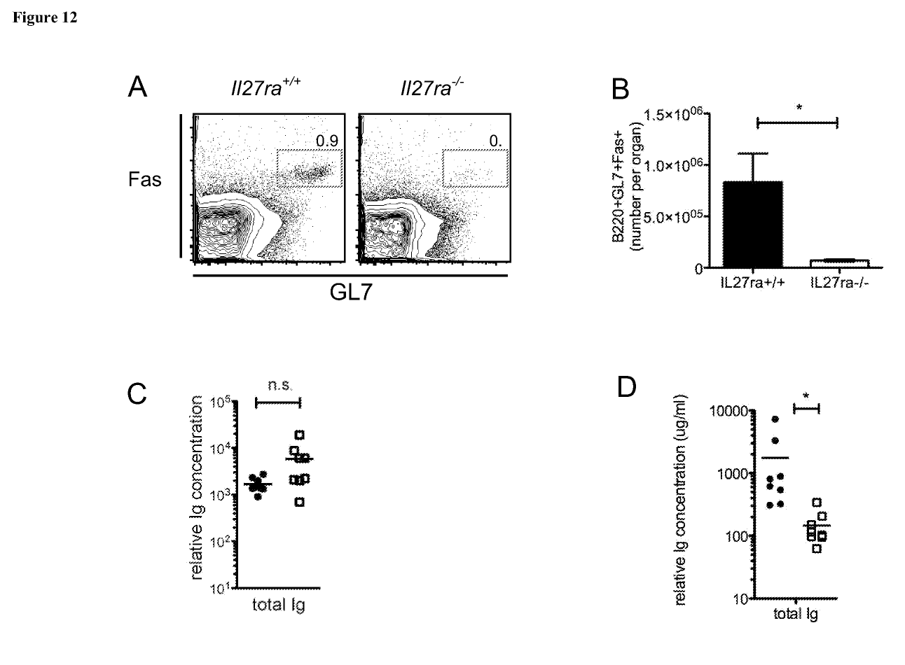

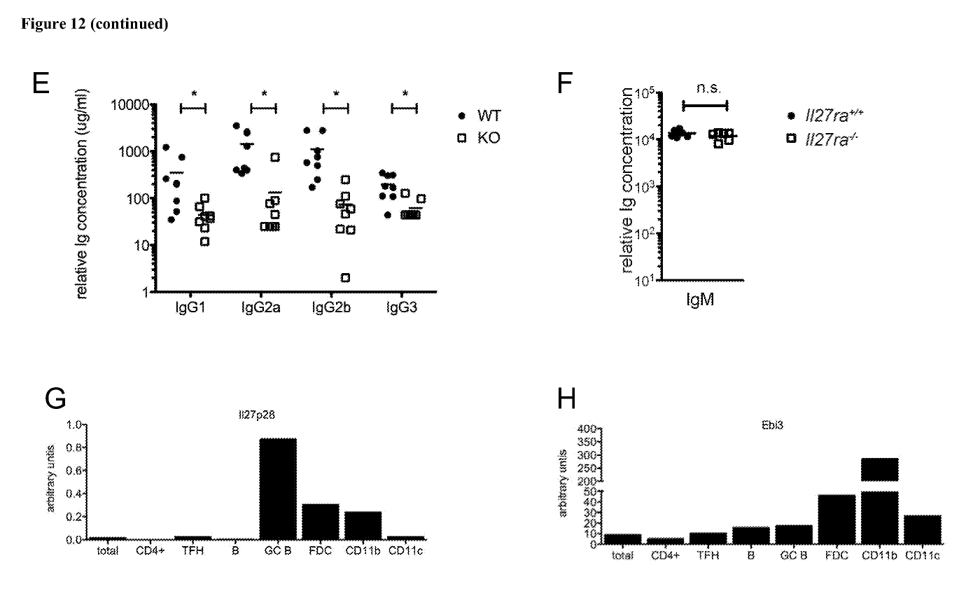

Reduced GC activity in the absence of IL-27Ra signaling. Mouse strains that have a deficiency of T_{FH} cells also display abortive GC reactions (de Vinuesa et al., 2000; Nurieva et al., 2008; Vogelzang et al., 2008). Thus, to confirm that T_{FH} function was diminished, the formation of GC in the spleens of immunized IL-27ra^{-/-} mice was examined. Flow cytometric analysis showed that, significantly fewer Fas⁺GL7⁺GC B cells were present in the absence of IL-27ra signaling (FIGS. 12A and B). Histological examination showed that while IL-27ra^{-/-} mice did develop some PNA⁺ GC structures, these were reduced in size and/or frequency compared to WT controls. The PNA positive area per spleen was objectively quantitated using image analysis software for each of 8 spleens per genotype and was found to be significantly reduced in IL-27ra^{-/-} mice.

To examine affinity maturation in IL-27ra^{-/-} mice, serum concentrations of high affinity antibodies to the immunizing hapten trinitrophenylated bovine serum albumen (TNP-BSA) were measured according to an established model (Roes and Rajewsky, 1993). Concentrations of antibodies to the immunizing hapten can be measured in the serum by coating ELISA plates with sparsely haptenated BSA molecules (TNP₂-BSA), to which only high affinity anti-TNP antibodies can bind. The level of total anti-TNP antibody (as detected using TNP₂₈-BSA) was similar in Il27ra^{-/-} and Il27ra^{+/+} sera (FIG. 12C). However, the level of high affinity anti-TNP antibodies was reduced in Il27ra^{-/-} mice compared to WT mice (FIG. 12D), indicating that affinity maturation is compromised in the absence of IL-27 signaling. IL-27ra-deficient mice had reduced levels of class switched high affinity antibodies including IgG1, IgG2a, IgG2b and IgG3 (FIG. 12E) but not IgE, an isotype that is actually inhibited by GC transcription factor Bcl-6 (Harris et al., 1999). In general, extrafollicularly derived antibody appeared to be unaffected by the loss of IL-27 signaling. Previous reports showed that IL-27ra^{-/-} mice displayed normal levels of total serum Ig, with the exception of IgG2a (Chen et al., 2000; Miyazaki et al., 2005). In line with these observations, it was found that the early IgM response to the T-independent antigen, TNP-Ficoll, was similar in Il27ra^{+/+} and Il27ra^{-/-} mice (FIG. 12F). This suggests that the GC response and resultant affinity maturation are selectively affected while extrafollicular Ig production is normal and the defect in Il27ra deficient mice is only illuminated when high affinity Ag-specific Ig is examined. Together, these data show that GC function along with T_{FH} cell number, are significantly reduced in the absence of IL-27 signaling.

It has been reported that IL-27 is expressed by activated monocytes, macrophages and dendritic cells in response to activation of TLRs or type 1 IFN and through the transcription factors NF-κB, IRF1 and 3 and PU.1 (Batten and Ghilardi, 2007; Nurieva et al., 2008). Since IL-27 is important for GC function, expression of IL-27 in cells that participate in the GC response, such as antigen-presenting follicular dendritic cells (FDC) which are central to the GC was examined. Various cell populations were sorted from TNP-OVA+CFA immunized mouse spleens and expression of IL-27p28 and IL-27ebi3 mRNA measured. The relevant entity in this context is IL-2′7p28, because EBI3 was recently reported to be shared with another cytokine, IL-35 (Collison et al., 2007; Niedbala et al., 2007), making its expression a less reliable indicator of IL-27 bioactivity. In agreement with previous reports (for review, see Batten and Ghilardi 2007), the two subunits are not co-ordinately regulated (FIGS. 12G & H). Both subunits of IL-27 were expressed by FDC as well as other CD11b⁺ cells present in CFA-activated spleens. However, it was surprised to note that the highest levels of IL-27p28 mRNA were observed in GC B cells (FIG. 12G). While expression of IL-27 subunits by B cells has been noted previously (Hasan et al., 2008), the physiological relevance of this has not yet been explored. This data strongly suggests that GC B cells induce the capacity to produce IL-21 in T_{FH} cells by secreting IL-27, but conclusive proof of this hypothesis will depend on the availability of a conditional IL-27p28 allele that can be specifically deleted in GC B cells. The expression of IL-27 in differentiated GC B cells, as well as FDC, may suggest that its ongoing expression within the GC structure is important for the activity of the T_{FH} cells.

### Example 4

### Effects of IL-27 on the Survival of T Cells Via Production of IL-21by CD4+ T-Cells

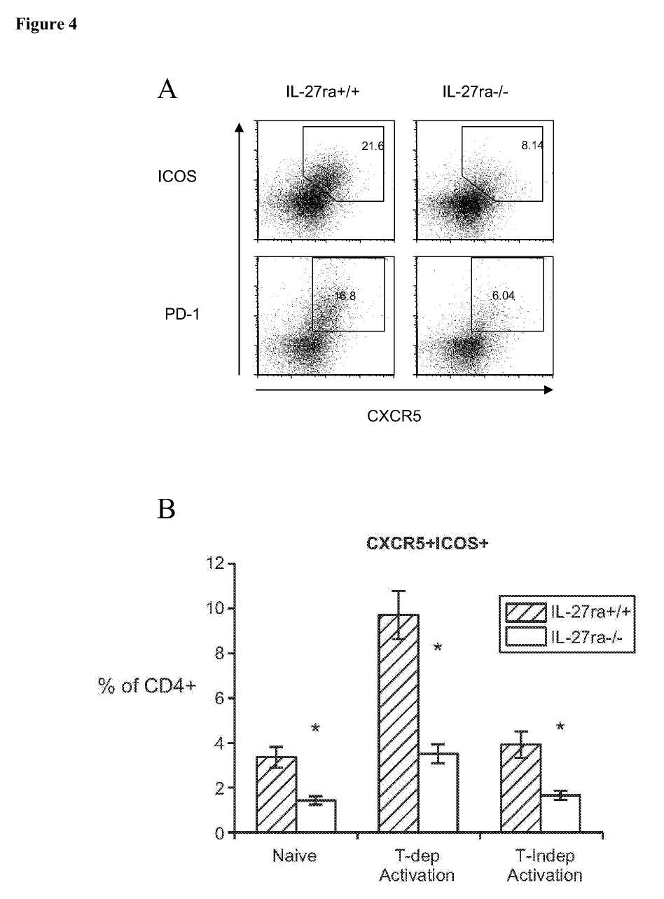

Flow cytometry of spleen and lymph node cells from immunized IL-27Ra^{-/-} mice revealed that they contain significantly reduced numbers of CXCR5+ICOS+ or CXCR5+PD1+ T_{FH} cells (FIGS. 4A and 4B), which could explain the reduction in GC number and size described above. IL-21 and IL-27 signaling deficient animals appear to have a similar defect in the T_{FH} and GC response. In vitro stimulation of CD4+ T-cells in the presence of rIL-27 results in induction of IL-21 mRNA and protein expression. Moreover, splenocytes from immunized IL-27Ra^{-/-} mice (KO) expressed significantly lower levels of IL-21 mRNA compared to wild-type mice, with CD4+ cells being the major source of IL-21. Interestingly, IL-27 shares a receptor with IL-6, a known potentiator of IL-21, and like IL-6, IL-27 activates STAT3. Because IL-21 is a TFH promoting factor, induction of IL-21 by IL-27 may be the mechanism by which IL-27 supports T_{FH} and GC activity. However, the development of T_{FH} cells depends on cues from B-cells. Therefore the reduction of T_{FH} number could be T-cell intrinsic or due to defects in the IL-27Ra−/− B-cells.

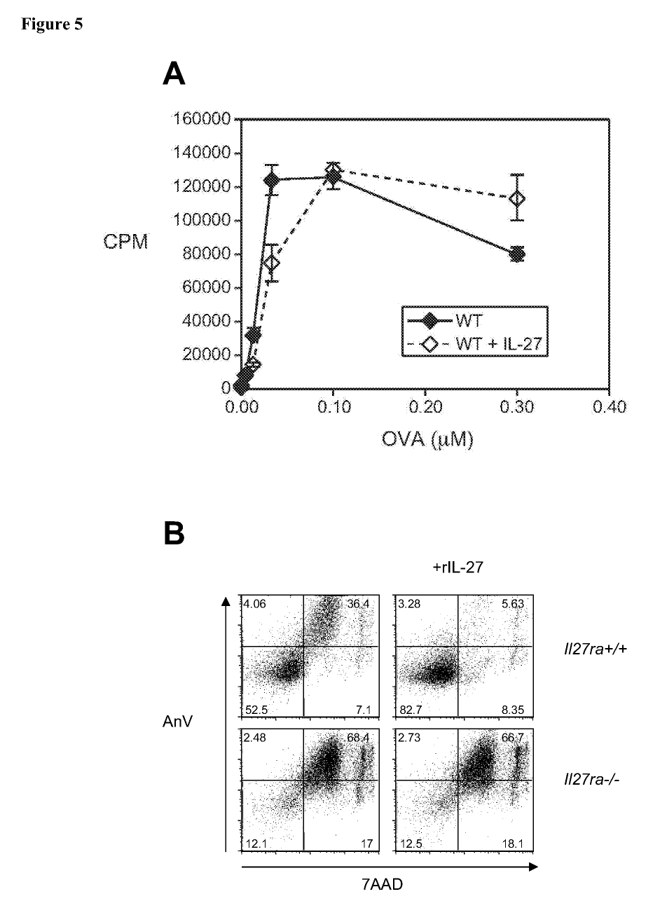

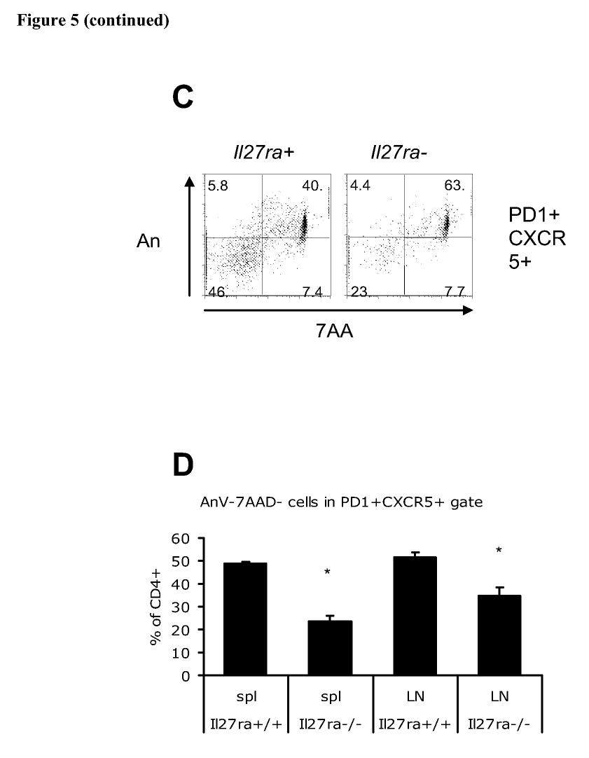

The effect of IL-27 on proliferation is highly dependent on the avidity of the TCR signal. At low doses of antigen, IL-27 suppresses proliferation of CD4+ cells. However, at higher concentrations the response was enhanced due not to increased cell divisions, as assessed by CFSE, but rather to enhanced survival of activated T-cells (FIGS. 5A and 5B). When the survival of CD4+ T-cells was investigated in vivo, no overall difference in viability between WT and IL-27Ra^{-/-} mice was observed. However, if the cells with a surface phenotype resembling T_{FH} cells (CXCR5+, PD1+, CD4+) were gated, a significant reduction in viable cells was noted in the absence of IL-27 signaling (FIGS. 5C and 5D). When CD4+ T-cells were stimulated in the presence of rIL-27 and various combinations of other cytokines and blocking antibodies, induction of CXCR5, ICOS or PD1 was not observed. Together, these data suggest that IL-27 supports the survival of T_{FH} cells rather than their differentiation:

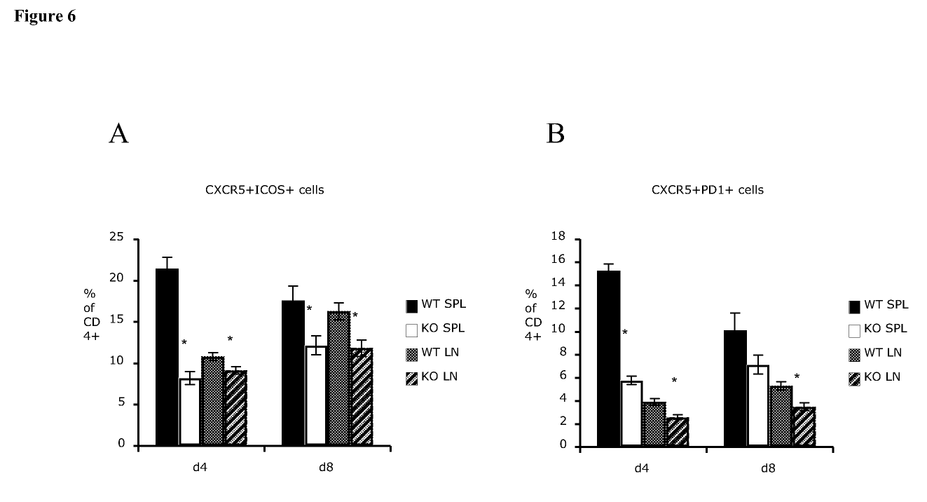

In a similar experiment, wild-type and IL-27Ra^{-/-} mice were immunized with TNP-OVA in CFA and spleens were harvested four days later. FACS plots were gated on CD4+/B220− cells and show the T_{FH} subset of T_{H} cells inside the gated region. FIG. 6A shows the average percentage of CXCR5+/ICOS+CD4+ T-cells. FIG. 6B shows the average percentage of CXCR5+/PD1+CD4+ T-cells.

The following experiment showed that IL-27 protects against activation-induced cell death. Wild-type or IL-27Ra^{-/-} T-cells were stimulated for three days in the presence of anti-CD3 (10 μg/ml) and anti-CD28 (1 μg/ml), in the presence or absence of murine IL-27. Cells were then rested for an additional three days. Subsequently recall proliferation was measured in response to increasing doses of plate-bound anti-CD3. Cells that were exposed to IL-27 for the preceding 6 days proliferated vastly better than those that were not. Next, microarray experiments were performed to identify those genes significantly affected on day six. IL-27 suppressed almost all granzymes as well as perforin, an important class of genes through which T-cells can kill target cells or each other in a tight tissue culture dish.

To determine whether IL-27 supported survival of highly stimulated cells, cells were first stimulated in vitro with anti-CD3/anti-CD28, then stained with Annexin V/7AAD to detect apoptotic cells. In this experiment, stimulation did not change the levels of PD1, CXCR5, or ICOS levels in vitro, and did not strongly induce the T_{FH} phenotype, as assessed by FACS. Stimulated wild-type mice and stimulated IL-27Ra−/− mice were immunized with antigen in CFA. Spleen and lymph nodes were harvested 4 days later. Apoptosis in the total T-cell gate (bar graph) and in the T_{FH} gate (FACS plots) was measured by Annexin V/7AAD staining. Those data indicate that IL-27Ra−/− tissues harbor more apoptotic cells.

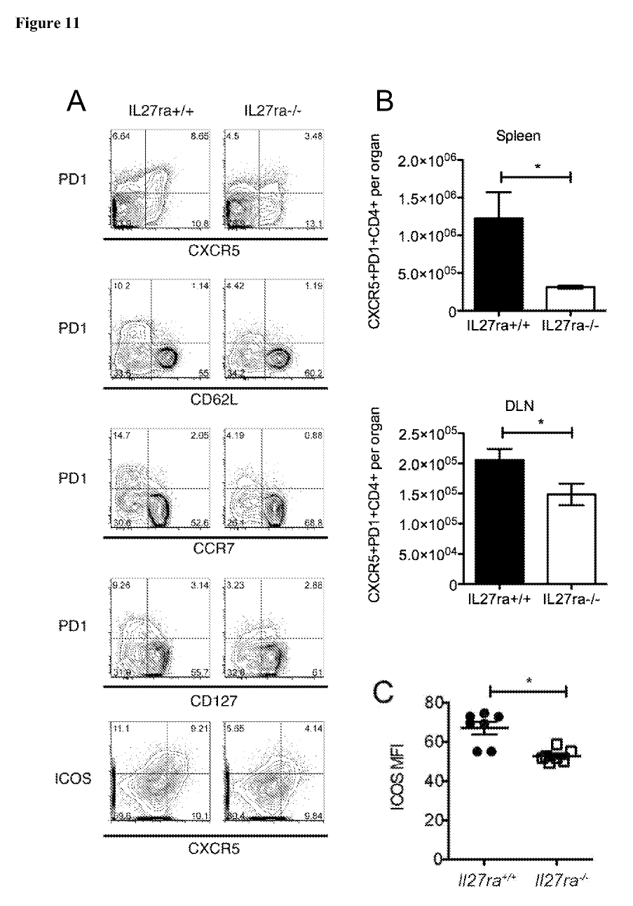

Reduced T_{FH} cell number in the absence of IL-27Ra signaling. Since IL-27ra^{-/-} mice had reduced IL-21 expression, and because IL-21 is both a differentiation factor for, and hallmark cytokine of, T_{FH} cells (King, 2009; Nurieva et al., 2008; Vogelzang et al., 2008), the size of the T_{FH} cell population in IL-27Ra knockout mice was examined. To this end, mice were immunized twice with TNP-OVA in Freund's complete adjuvant. Cell phenotypes in spleen and lymph nodes were analyzed 7 days after the second immunization. A statistically significant reduction in both the percentage and absolute number of PD1⁺CXCR5⁺CD4⁺ T cells was observed in the spleens and draining LN of IL-27ra^{-/-} mice (FIGS. 11A and B). To ensure proper discrimination between T_{FH} and activated T cells, cells were stained with additional markers, showing that IL-27ra^{-/-} mice have a reduction in CXCR5⁺, PD1⁺, ICOS⁺, CCR7^{lo}, CD62L^{lo}, CD127^{lo} cells (FIG. 11A), a population matching the published phenotype of T_{FH} cells. In addition to having diminished T_{FH} cell numbers, the Il27ra^{-/-} mice displayed diminished ICOS levels on the remaining cells within the PD1⁺CXCR5⁺ gate (FIG. 11C), which may reflect reduced B cell helper function in the few cells with a T_{FH} phenotype. Mice were immunized as described above.

Transfers of TCR transgenic T-cells (OT-II TCR Tg.Thy1.1+ congenic) into IL-27Ra^{-/-} and WT recipients are performed, and the number of TFH cells present after immunization with 30 μg OVA in CFA is quantitated by flow cytometry as described in Table 1 and using Thy1.1+ antibody to detect transferred T-cells. IL-27Ra-sufficient OT-II T-cells elicit equally potent GCs in IL-27Ra^{-/-} and WT hosts, suggesting that the remainder of the immune response, including B-cells, is intact in IL-27Ra^{-/-} mice, pointing to a T_{FH} intrinsic defect being responsible for the reduced GC responses in these mice.

Second, bone marrow (“BM”) chimera experiments are performed in IL-27Ra^{-/-}.TCRa/b^{-/-} and IL-27Ra^{-/-}.muMT^{-/-} mouse lines. Using BM from these mice in different combinations mice with T or B-cell specific deletions of IL-27Ra are generated as indicated in Table 2 below. Those animals are then immunized with TNP-OVA as described above and the efficiency of GC reactions tested after 7 and 14 days by flow cytometric analysis, histology and detection of high affinity anti-TNP antibodies in the serum by ELISA. The production of cytokines (IL-21, IL-10, IFNg, IL-4 and IL-17) by CD4+ T-cells is assessed by intracellular staining and flow cytometry after 4 hours of restimulation with PMA/ionomycin in the presence of BFA.

GC development is investigated in IL-21R^{-/-}.IL-27Ra^{-/-} double knockout mice. Neither IL-21R^{-/-} mice nor IL-27Ra^{-/-} mice have a complete lack of T_{FH} cells or GC. Therefore, if IL-21 and IL-27 work in the same pathway, the phenotype of the mice should be similar to either of the single knockouts. However, if IL-21 and IL-27 independently support the GC response then the double knockout should develop a more severe defect. Next, mixed BM chimeras are constructed. BM chimeras reconstituted with IL-27Ra^{-/-} cells alone have a paucity of GC after immunization. However, mixed WT and IL-27Ra^{-/-} BM reconstitution results in GC responses comparable to WT, suggesting that a factor produced by WT-cells compensates for the defect in IL-27Ra^{-/-} cells. To determine whether this factor is IL-21, BM chimeras are generated using IL-27Ra^{-/-} cells mixed with IL-21^{-/-} cells. Groups of mice are reconstituted with: (i) WT only; (ii) IL-27Ra^{-/-} only; (iii) IL-27Ra″+WT; and (iv) IL-27Ra^{-/-}+IL-21^{-/-} bone marrow. In the IL-27Ra^{-/-}+IL-21^{-/-} chimera, cells that do express IL-27Ra will not be able to produce IL-21 and will only be able to respond to the minimal amounts of IL-21 produced by IL-27Ra−/− T-cells (FIG. 6). If the IL-21^{-/-} cells can no longer provide a compensatory signal, GC development is reduced compared to WT reconstituted mice. Finally, IL-21 production is reconstituted in IL-27Ra^{-/-} mice using a retroviral IL-21 expression vector and the GC response is compared to mice infected with control retroviral vectors.

The GC is a highly antigen rich and stimulatory environment that may be conducive to activation induced cell death (AICD) of CD4+ T-cells. Indeed, flow cytometry shows that a large proportion of T_{FH} cells recovered from immunized mice take up dead cell stains such as 7AAD and PI (FIG. 6B). Therefore, the ability of rIL-27 to reduce apoptosis levels in in vitro death assays in which cells are treated with anti-Fas crosslinking antibodies or with anti-CD3 in the absence of costimulation is tested.

IL-21 activates Pi3K and has been shown to promote survival of T_{FH} cells. R. I. Nurieva et al., Immunity 29(1):138-49 (2008). To determine whether the survival effects of IL-27 also depend on the upregulation of IL-21, the following experiments are performed. First, in vitro stimulation assays to test the ability of rIL-27 to promote the survival of IL-21−/− CD4+ T-cells are performed. If IL-27 promotes survival via IL-21 then no effect is observed in IL-21 deficient cultures. To determine whether this is a Pi3K-mediated effect, the ability of Pi3K inhibitors to block the effects of IL-27 and IL-21 is tested. Next, the viability of T_{FH} cells is assessed in IL-27Ra−/− mice retrovirally transfected with IL-21 expression vectors, compared to wild-type and IL-27Ra−/− mice infected with control vectors. If IL-21 overexpression compensates for the defect in IL-27 signaling, then IL-27-induced production of IL-21 is necessary for the survival of T_{FH} cells. If IL-21 overexpression does not compensate for the defect in IL-27 signaling, IL-27 likely has a distinct role in T_{FH} survival.

If IL-27 plays a distinct role in T_{FH} survival, the ability of rIL-27 stimulation of purified CD4+ cells in vitro to induce expression of other survival factors is assayed. Survival factors assayed include Bcl-6, a pro-survival transcription factor that is found specifically in the T and B-cells of the GC, as well as members of the Bcl-2 family that play central roles in cellular survival control, such as, for example, Bcl2, Bcl_{XL} and Bax. In parallel experiments, the expression of the death receptors of the TNF family including Fas, Trail-receptors (DR4 and DR5), and TNFR1 are assayed. Expression of those factors is assessed by RT-PCR. Where appropriate antibodies are available, protein expression is assessed in parallel by flow cytometry or Western blot of IL-27-stimulated CD4+ mouse T-cells, as well as from the spleen and LN from immunized IL-27Ra^{+/+} and ^{-/-} mice.

Finally, aged IL-27Ra−/− mice display a generalized reduction in B-cell memory phenotype (CD44+) CD4+ cells. Since cells with a T_{FH} surface phenotype fall into this category, memory T-cell responses in vivo are investigated to determine whether IL-27 supports the survival of antigen-stimulated T-cells as a whole. Those experiments will require the transfer of TCR transgenic CD4+ T-cells in order to follow the antigen-specific response. OT-II TCR Tg mice express a TCR that recognizes OVA peptide. Those mice have been crossed with Thy1.1+ congenic mice so that the cells can be identified using antibodies when transferred into Thy1.2 host mice. In these experiments, 1×10⁶ CFSE labeled OT-ILIL-27Ra^{+/+} or cells are transferred into each of 20 recipients per group. To reduce the persistence of antigen, as may occur with immunization in lipid-based adjuvant, the mice are immunized with peptide-loaded LPS matured bone marrow derived dendritic cells. The primary response are assessed 7 days later by harvesting the spleens from half the mice and performing flow cytometry to determine proliferation of Thy1.1+ cells, and to assess the differentiation and survival of Thy1.1+T_{FH} cells. Since T_{FH} cells are a type of antigen-experienced cell they may depend on additional cytokine growth factors such as IL-7 and IL-15. Therefore, the expression of IL-7R, IL-15R and CD122 (IL-2Rb) is measured on total CD4+ T and TFH cells. The remaining mice are given a second immunization 6 weeks after the first and the secondary response is assessed 7 days later in a similar way.

TABLE 2
Establishment of BM chimeras to generate cell
specific IL-27Ra mutations.

| | | CD4+ | B-cell | | |
|---|---|---|---|---|---|
| | | IL- | IL- | Other | |
| BM 1 | BM 2 | 27Ra | 27Ra | leukocytes | result |
| IL-27Ra^{-/-}. | uMT | WT | KO | WT or KO | B-cell specific |
| TCRa/b | | | | | deletion |
| TCRa/b | IL- | KO | WT | WT or KO | T-cell specific |
| | 27Ra.uMT | | | | deletion |
| TCRa/b | uMT | WT | WT | WT | WT control |
| IL-27Ra^{-/-}. | IL- | KO | KO | KO | KO control |
| TCRa/b | 27Ra.uMT | | | | |

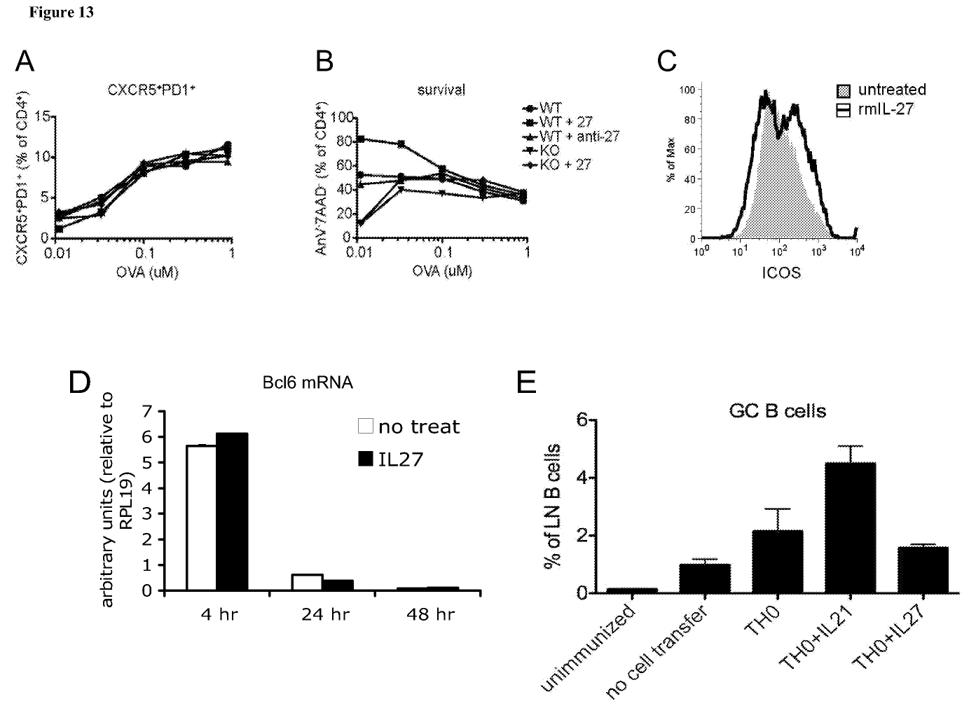

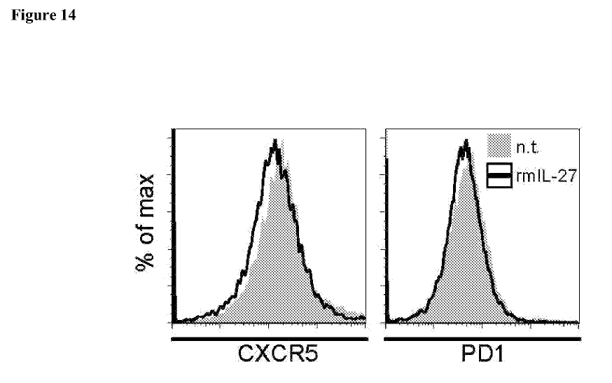

IL-27 supports survival rather than differentiation of T_{FH} cells. IL-27ra-deficient mice have a pronounced defect in IL-21 expression and T_{FH} cell number, suggesting that IL-27 is important for either the differentiation or maintenance of T_{FH} cells. To test whether IL-27 stimulation of CD4⁺ T cells directly induced phenotypic characteristics of T_{FH} cells in vitro experiments using total splenocytes from DO11.10_Tg.Rag2^{-/-} mice were performed. All CD4⁺ T cells in these animals are naïve and recognize the peptide OVA₃₂₃₋₃₃₉ presented on antigen-presenting cells (“APC”). A range of antigen concentrations were used for stimulation and, in line with a previous report demonstrating that the strength of the antigen signal affects T_{FH} differentiation (Fazilleau et al., 2009), increasing concentrations of OVA peptide stimulation led to elevated PD1 and CXCR5 levels (FIG. 13A). However, neither the addition of rmIL-27, nor the loss of IL-27 signaling, during OVA stimulation altered the expression of T_{FH} markers PD1, CXCR5, or the T_{FH} associated transcription factor Bcl-6 (FIG. 14 and FIGS. 13A and D), even though rmIL-27 stimulation increased the percentage of ICOS⁺ cells (FIG. 13C) in accordance with a recent report [Pot et al. 2009].

To determine functionally whether IL-27 signaling enhances the B cell helper activity of CD4⁺ T cells, CD4⁺ T cells from OTII TCR Tg mice were stimulated ex vivo with the cognate peptide OVA₃₂₃₋₃₃₉, in the presence of either no additional cytokines, rmIL-21 or rmIL-27 under T_{H}0 conditions for 5 days. Equal numbers of the activated cells were then adoptively transferred to naïve syngeneic hosts, which were subsequently immunized with OVA in IFA. As described previously (Nurieva et al., 2008), in vitro stimulation in the presence of rmlL-21 resulted in enhanced development of GC in the adoptive hosts (FIG. 13E), however, pre-treatment with rmlL-27 was not able to enhance B helper activity under these conditions. Together, these data suggest that IL-27 is not able to directly induce maturation of T_{FH} cells. However, the addition of rmIL-27 to in vitro cultures as in FIG. 14 enhanced the survival of these strongly stimulated T cells (FIG. 5B and FIG. 13B). To investigate whether the altered cell survival observed in vitro was reflected by changes in T_{FH} cell survival in vivo, the percentage of viable (Annexin V^{neg}7AAD^{neg}) T_{FH} cells in immunized IL-27ra^{+/+} and IL-27ra^{-/-} mice was examined. At 4 days post immunization, while overall CD4+ T cell viability was similar between WT and IL-27ra^{-/-} mice, the viability of T_{FH} cells was significantly reduced in the spleen and draining LN of IL-27ra^{-/-} mice (FIGS. 5C and 5D). Together these data suggest that IL-27 is important for the maintenance rather than the differentiation of T_{FH} cells.

### Example 5

### Effect of IL-27 on B-Cells and the GC Reaction

B-cells express the IL-27 receptor and STAT1 is activated by rIL-27 stimulation of B-cells. Loss of IL-27 signaling to B-cells could therefore contribute to the GC defect observed in IL-27Ra-1−/− mice. Indeed, IL-27 promotes IgG2a production. This effect on IgG isotype levels differs from that of IL-21, which is predominantly important for switching to IgG1. Thus, the effects of IL-27 on class switching are unlikely to be attributable to IL-21 induction, suggesting a direct effect on B-cells. The well characterized SW_{HEL} BCR transgenic mice are used to study antigen-specific B-cell responses as well as class switching and the level of somatic hypermutation that occurs, although normal selection of high affinity clones does not occur, probably because the affinity of the BCR for HEL is already extremely high.

The SW_{HEL} and IL-27Ra^{-/-} mouse lines are crossed. B-cells from SW_{HEL}.IL-27Ra^{-/-} or ⁺¹+ mice are transferred to WT CD45.1 congenic recipients immunized with HEL conjugated to ovalbumin. The differentiation of the antigen-specific B-cells into GC B-cells is examined by flow cytometry and antibody class switching is assessed by serum ELISA. The development of T_{FH} cells, which depends on communication with B-cells, is also assessed by flow cytometry. In a second set of experiments, IL-27Ra-sufficient and IL-27Ra-deficient SW_{HEL} B-cells are co-transferred along with OT-II TCR T-cells. Those mice are immunized with OVA-conjugated HEL so that both the T and B-cell responses are assessed in an antigen specific way. The differentiation of the cells is examined by flow cytometry and the localization of the cells is visualized by fluorescent histology using antibodies that detect the antigen specific B and T-cells.

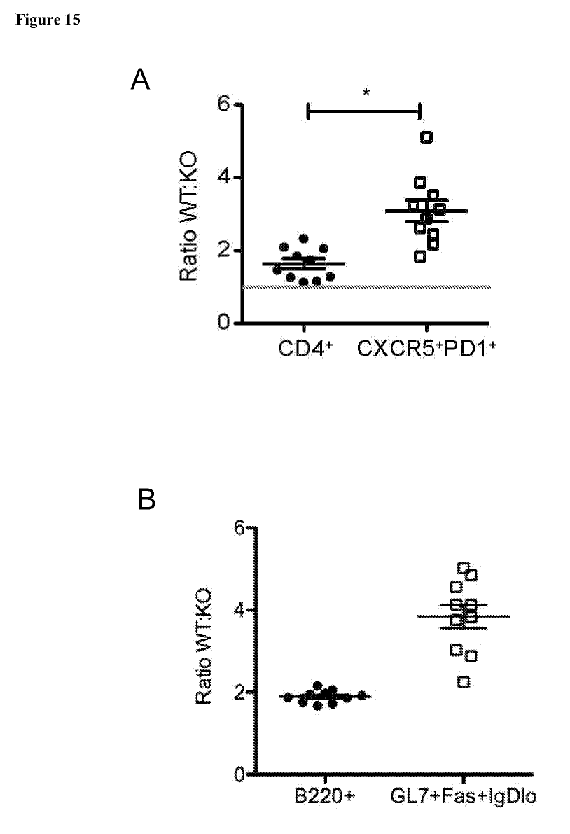

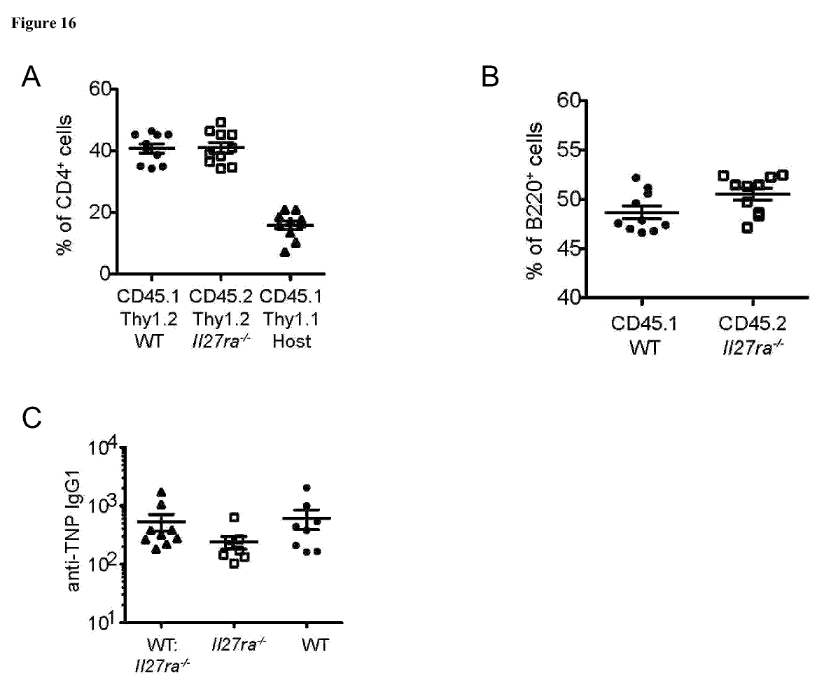

T and B cell autonomous defects contribute to the GC phenotype observed in IL-27ra^{-/-} mice. The observation that IL-27 promotes IL-21 expression by, and survival of, T cells suggested that the defect in T_{FH} cell number and GC function in IL-27ra^{-/-} mice could result from a T cell intrinsic defect. However, the IL-27 receptor is expressed by other cells, including B cells, and thus it remained possible that the T_{FH} defect in IL-27ra^{-/-} mice was indirect. To discriminate these two possibilities, mixed bone marrow (BM) chimeras were constructed using Il27ra^{-/-}. (CD45.2⁺, Thy1.2⁺) BM mixed at a 1:1 ratio with congenic Il27ra^{+/+} (CD45.1⁺, Thy1.2⁺) BM and transferred into lethally irradiated Il27ra^{+/+}CD45.1⁺ Thy1.1⁺ triple congenic (TCM) hosts such that Il27ra^{-/-} donors, WT donors and remnant WT host T cells could each be differentiated in the reconstituted chimeric mouse. In such mice, both WT and Il27ra^{-/-} cells have the same exposure to the mixed WT and KO antigen presenting cells. FACS analysis of the blood of reconstituted chimeric mice revealed similar contribution of both genotypes to T and B cell compartments (FIGS. 16A and B), suggesting that absence of the Il27ra does not confer impaired repopulation capacity. The chimeric mice were immunized twice, and 7 days after the second immunization the contributions of the WT and IL-27ra^{-/-} cells to the splenic total CD4⁺, total B220⁺, CD4⁺CXCR5⁺PD1⁺ T_{FH} and GL7⁺Fas⁺IgD^{lo}GC B cell populations were analyzed by flow cytometry. In the total CD4⁺ gate, WT cells contributed with somewhat increased frequency, producing a WT:KO ratio of 1.644+/−SEM of 0.14 (FIG. 15A). However, consistent with the finding that IL-27 promotes survival of T₁ cells, WT T cells clearly contributed disproportionately to the PD1⁺CXCR5⁺ T₁ gate, producing a WT:KO ratio of 3.08+/−0.3 (FIG. 15A). This data suggests that IL-27ra^{-/-}. T cells have an intrinsic defect in T_{FH} development and/or maintenance which cannot be compensated for by the presence of WT APC and B cells. Since activated bystander WT cells in the chimeric animals were capable of producing IL-21, the T_{FH} defect observed in IL-27ra^{-/-} mice likely is not solely due to reduced IL-21 expression. Since the T_{FH} compartment of the mixed chimeric mice was comprised mainly of WT cells, GC function was restored and the levels of high affinity class-switched Ig were comparable with mice reconstituted with 100% WT cells (FIG. 16C).

Since B cells also express the IL-27 receptor and IL-27 has been shown to promote isotype switching and B cell proliferation in vitro (Larousserie et al., 2006; Pflanz et al., 2004; Yoshimoto et al., 2004) a defect in IL-27 signaling to B cells could also contribute to GC dysfunction. Analysis of the B cell population in the chimeric mice showed that, similar to the T cell compartment, WT cells contributed with a slightly increased frequency to the total B220+B cell pool with the ratio of WT:KO B cells being 1.89±SEM of 0.047 (FIG. 15B). However, the WT:KO ratio in the GL7⁺Fas⁺IgD^{lo}GC B cell gate was 3.84±0.28 (FIG. 15B). This suggests that in addition to the defect in T_{FH} cells, IL-27ra^{-/-} mice have a B cell intrinsic defect in GC B cell development and/or maintenance.

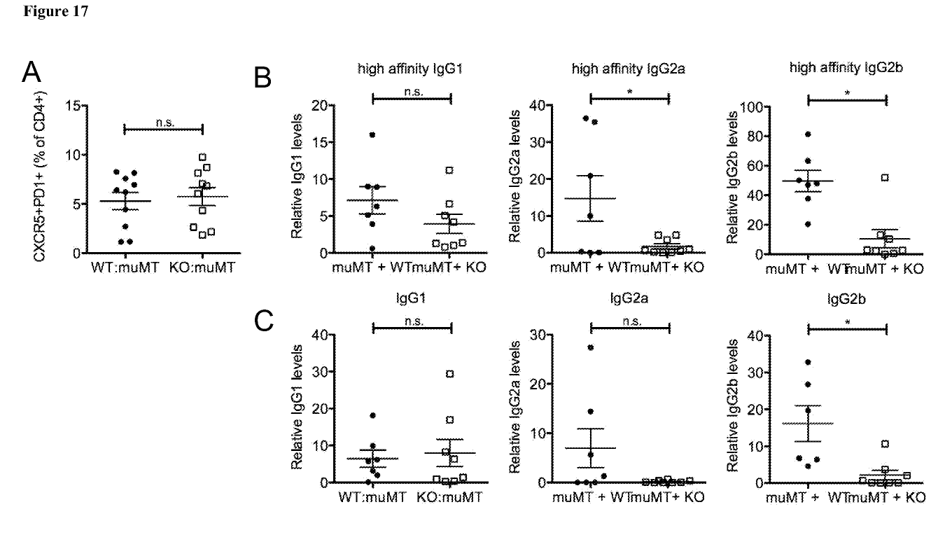

To further investigate the B cell specific effects of IL-27 signaling, mixed BM chimeras with either IL-27ra^{+/+} or IL-27ra^{-/-} BM mixed with BM from B cell deficient μMT deficient mice were constructed. In such chimeric mice, all B cells were derived from the IL-27ra^{+/+} or IL-27ra^{-/-} graft, whereas all other cell types represent an approximately equal mixture of μMT (IL-27ra WT) and IL-27ra^{+/+} or IL-27ra^{-/-} genotypes. In this system, the loss of IL-27ra specifically in the B cells produced similar proportions of T_{FH} cells compared to mice where WT B cells were present (FIG. 17A), suggesting that loss of IL-27 receptor on B cells does not inhibit the differentiation of T_{FH} cells and confirming that the T_{FH} defect in IL-27ra^{-/-} mice is T cell intrinsic. However, loss of the IL-27 receptor on B cells resulted in attenuated development of high affinity IgG1 (n.s.), IgG2a (p=0.031) and IgG2b (p=0.0011) antibodies (FIG. 17B). A reduction in the overall level of IgG2a and IgG2b was also observed (FIG. 17C). Serum titers of other isotypes or of the total Ig were unchanged between IL-27ra^{-/-}:μMT and IL-27ra^{+/+}:μMT chimeras (data not shown). Taken together, the data shown in FIGS. 15B and 17 indicates that deletion of IL-27ra specifically in the B cell compartment decreases GC B cell number and the production of certain isotypes of high affinity antibody. However, since overall antibody production is affected, this defect may not be confined to the GC response. It appears that B cell loss of IL-27 responsiveness contributes to the humoral defect in IL-27ra^{-/-} mice, but that the defect in T_{FH} cell survival is T cell intrinsic and independent of the effects of IL-27 on B cells.

Conclusions. Thus, IL-27 likely supports the GC response via several mechanisms. First, IL-27 produced by FDC and GC B cells induces IL-21 production in T_{FH} cells, suggesting that IL-27 is important for the initial induction of IL-21 expression. Production of IL-21 subsequently initiates an autocrine feedback loop in T_{FH} cells by an unknown mechanism. Second, IL-27 supports T_{FH} survival. T_{FH} cells are highly activated, express high levels of Fas and are exquisitely sensitive to activation induced cell death (Marinova et al., 2006). T_{FH} cells also undergo enhanced apoptosis when IL-27Ra is genetically ablated. Finally, IL-27 has direct and non-redundant functions on B cells. Taken together, this data suggests that therapeutic targeting of IL-27 may be useful in disorders characterized by excessive germinal center formation and high affinity autoantibody production, such as systemic lupus erythematosus.

### Example 6

### IL-27's Effects on Progression of Immunopathic Diseases Dependent on Both T and B-Cells

GCs are thought to be important for the production of pathogenic antibodies in certain autoimmune diseases including, for example, SLE. Although there are a number of mouse models of lupus, the Sanroque model from the laboratory of associate investigator Dr. Carola Vinuesa is of particular relevance to the present project because the lupus phenotype results from aberrant T-cell help for B-cells in the GC and is transmissible by a single gene mutation in C57BL6 mice. IL-27Ra.Sanroque cross mice are generated to compare the course of disease to IL-27Ra-sufficient Sanroque mice. At 4, 6, 8, 12 and 22 weeks serum is collected for analysis of hypergammaglobulinemia (including assessment of isotypes) and ANA immunofluorescence on a Hep-2 substrate. Those time points are selected to cover the spectrum of disease in Sanroque mice progressing from minimal disease symptoms to onset of ANA production in the majority of mice. C. G. Vinuesa et al., Nature 435(7041):452-58 (2005). Ten mice per genotype are sacrificed age 6 weeks, 10 weeks and 20 weeks to assess autoimmune manifestations such as glomerulonephritis, necrotosing hepatitis and anaemia.

IL-27Ra−/− mice have already been shown to be resistant to the PGIA model of arthritis. This model is replicated to examine whether GC defects are observable by histology. Flow cytometry is used to assess the viability of T_{FH} cells in IL-27Ra−/− compared to +/+ mice. BALB/c mice are susceptible to disease in this model. IL-27Ra−/− mice backcrossed to BALB/c mice for more than 10 generations are used for those experiments. Reconstitution experiments are performed with retroviral expression of IL-21 to determine whether the PGIA defect is ameliorated by the expression of IL-21.

### Example 7

### Patients with SLE Display an IL-27 Gene Signature Characteristic of the Disease

To confirm the role IL-27 played in human SLE, an IL-27 gene signature was identified and its presence detected in PBMC samples from lupus patients. First, it was determined qualitatively that all cell types contained in human PBMC, including B-cells, CD11b+myeloid cells, and CD4+ and CD8+ cells can respond to recombinant human IL-27, which induces phosphorylation of STAT1 and STAT3.

Next, samples of PBMC were obtained from eleven human donors, and time course stimulations of each sample were performed with IL-27 or IFNα. Response to IL-27 was determined by quantitative RT-PCR for expression of T-bet, a transcription factor known to be induced by IL-27. The gene expression signature described infra. The 16 hour time points of the best six donors were used to perform microarray analysis. GeneChip® Human Genome U133 Plus 2.0 array from Affymetrix, Inc. (catalog no. 900470) was used.

Genes having an unadjusted p-value<0.001 and >two-fold higher expression in the IFNα-treated sample compared to the control sample were selected as initial IFNα signature genes. These were further filtered to remove genes that showed an adjusted p-value<0.05 in the IL-27 treatment, yielding an initial list of 358 probes (275 genes). Genes having an unadjusted p-value<0.001 and > two-fold higher in the IL-27-treated sample than the control sample were selected as initial IL-27 signature genes. This list was filtered to remove genes with an adjusted p-value<0.05 in the IFNα treatment, yielding a list of 434 probes (313 genes).

Because both cytokines signal through the transcription factor STAT1, an overlap between the initial IFNα and IL-27 signatures was expected. After reviewing the genes within the initial IL-27 signature, however, IFNα response genes previously identified in the literature were removed from the IL-27 signature to provide a pure IL-27 gene set free of contamination with IFNα induced genes.

Expression of IL-27 signature genes were profiled using PBMC (Peripheral Blood Mononuclear Cells) RNA samples from both healthy controls and lupus patients. The signature genes were generally higher in lupus patients than in healthy controls suggesting an association between IL-27-responsive genes and the disease. The IL-27 gene signature was further characterized using the comparison of lupus and healthy controls. Genes significantly up-regulated at an adjusted p-value<0.001 were selected to give a final IL-27 gene signature of 31 probes (21 genes) shown in FIGS. 19A and 19B.

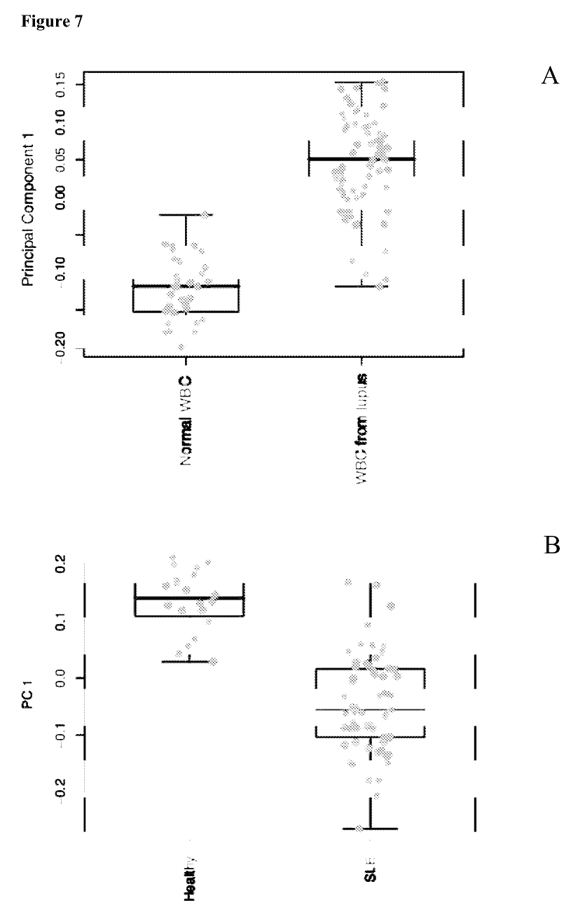

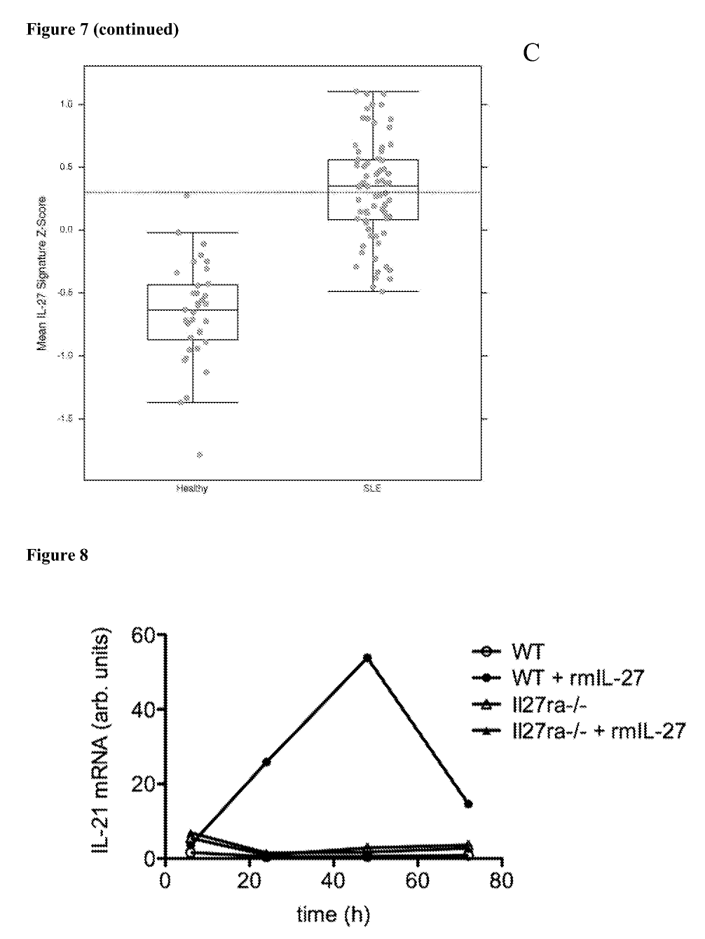

Principal component analysis, a statistical method used to identify dimensions in which data clusters segregate from each other, confirmed an unambiguous, statistically significant difference between lupus patients and healthy controls with respect to expression of genes in the IL-27 signature (FIG. 7A). Similar results were obtained using clinical samples from a different cohort of lupus patients and healthy controls, further strengthening the case for an IL-27 signature in lupus (FIG. 7B).

For each probe in the IL-27 gene signature (31 probes in FIGS. 19A and 19B), a z-score across the SLE and healthy control gene expression data was calculated. The z-scores from each set of genes were averaged to create an aggregated gene expression statistics. A cutoff at the mean plus two standard deviations of the normal patient value (approximately the 95^{th} percentile in a normally distributed sample) was calculated. Using these cutoffs, a sub-population of IL-27 high patients was identified.

### Example 8

### Effects of IL-27 Antagonists on Ameliorating Symptoms of SLE in a Mouse Model

Exemplary IL-27 antagonists directed against IL-27 or the IL-27 receptor are tested in two different mouse models of systemic lupus erythematosus (“SLE”): (1) F1 hybrids of NZB/NZW mice; and (2) BALB/c mice injected with pristane intraperitoneally.

A mouse model of systemic lupus erythematosus (“SLE”) arising spontaneously in F1 hybrids of two mouse strains: the autoimmune New Zealand Black (“NZB”) and the phenotypically normally New Zealand White (“NZW”) have been described. E. L. Dubois et al., J. Am. Med. Assoc. 195(4):285-89 (1966). NZB/NZW mice develop severe systemic autoimmune disease more fulminant than that observed in the parental NZB strain. These mice manifest various immune abnormalities, including antibodies to nuclear antigens and subsequent development of a fatal, immune complex-mediated glomerulonephritis with female predominance, remarkably similar to SLE in humans.

Intraperitoneal administration of a single injection of pristane (2,6,10,14-tetramethylpentadecane) to BALB/c mice before the injection of hybridoma cells is commonly used to obtain monoclonal antibody-enriched ascitic fluid. In addition to its effects on hybridoma cell growth, however, pristane also induces the production of polyclonal IgG autoantibodies to Su, U1RNP, U2RNP, U5RNP and/or Sm. Anti-Su antibodies appear as early as 1-2 months after a single 0.5 ml injection of pristane, followed by anti-U1RNP and anti-Sm antibodies after 2-4 months. Within six months of injection, the majority of mice develop anti-Su, anti-U1RNP, anti-U2RNP, anti-Sm, and in some cases, anti-U5RNP. Thus, injection of pristane induces lupus-like autoimmunity in a strain of mouse not normally prone to autoimmune disease.

Mixed-gender groups of twenty 6-8 week old age-matched NZB/NZW mice or BALB/c mice previously injected with 0.5 ml of pristane are obtained. Serum samples are obtained from each mouse and levels of serum autoantibodies are assessed by Western blot or by enzyme-linked immunosorbent assays (“ELISA”) against a panel of autoantigens characteristic of SLE using standard methods known in the art. Groups of each mouse strain are treated IP once per week for ten weeks or treated three times per week (e.g., 150 mg per mouse) with (1) murinized anti-IL-27p28 antibody mAb 4066; (2) murinized anti-IL-27Ra antibody mAb 2918; or (3) a vehicle control (for example, an anti-gp120 antibody). Each antibody is tested at the following doses: (1) 1 μg/kg; (2) 10 μg/kg; (3) 100 μg/kg; (4) 250 μg/kg; (5) 500 μg/kg; or (6) 1 mg/kg. Serum samples are obtained weekly from each mouse and levels of serum autoantibodies are assessed by Western blot or ELISA against the same panel of autoantigens tested prior to treatment. Levels of serum autoantibodies in animals treated with murinized anti-IL-27p28 antibody mAb 4066 or murinized anti-IL-27Ra antibody mAb 2918 are compared to those receiving the vehicle control.

Alternatively, expression levels of IL-10 and/or IL-21 are monitored by RT-PCR before and after injection with murinized anti-IL-27p28 antibody mAb 4066 or murinized anti-IL-27Ra antibody mAb 2918. Levels of IL-10 and IL-21 mRNA in animals treated with either antibody are compared to those receiving the vehicle control. After each week of treatment, two NZB/NZW mice are killed, the kidneys removed, and immune complex deposition assessed by immunohistochemistry with appropriate antibodies. Levels of renal immune complex deposition (i.e., proteinuria) and associated glomerulonephritis in animals treated with either antibody are compared to those receiving the vehicle control. Alternatively, serum autoantibodies can be determined from a bleed without the need to sacrifice the animals.

### Materials and Methods.

Real time RT-PCR. Total RNA from FACS sorted or cultured cells was isolated with the RNeasy kit using on-column DNAse I digestion (Qiagen, La Jolla, Calif.). Taqman® quantitative RT-PCR was done according to the instructions of the manufacturer (Applied Biosystems, Redwood City, Calif.). A Roche Lightcycler480 instrument was used in the case of human samples. For each sample, triplicate test reactions and a control reaction lacking reverse transcriptase were analyzed for expression of the gene of interest and results were normalized to those of the ‘housekeeping’ ribosomal protein L19 (RPL19) mRNA or hGAPDH. Arbitrary units given are the fold change relative to RPL19 (mouse) or GAPDH (human) and multiplied by 1000. Primer sequences for each target are provided in Table 3 below.

TABLE 3
Primer sequences used in RT-PCR.

| | | |
|---|---|---|
| gene | primers | probe |

| | | |
|---|---|---|
| mIl21 | CTTCCCGTGTCAGGGATT | AGCCACAGCTTGAGAAGCACCAGA |
| | TCACAGTTGGGCAATAAGATG | |
| mIl27p28 | TCAGGTGTCATCCCAAGTGT | GGTAGGTATAGAGCAGCTGGGGCCAG |
| | GACAAGCTCCAGGGAGTGA | |
| mEbi3 | GGCCTGTCCTGAGTCTGAATA | CTTTCCATGTACTGGGCTGCTCCG |
| | AGTCAAGTGAATTATCCAGTGCTT | |
| mRpl19 | ATCCGCAAGCCTGTGACTGT | TTCCCGGGCTCGTTGCCG |
| | TCGGGCCAGGGTGTTTTT | |
| mBcl6 | Inventoried Taqman ™ assay | |
| | Mm00477633_m1 | |
| hGAPDH | CTCTGCTCCTCCTGTTCGAC | Roche UPL probe #60 |
| | ACGACCAAATCCGTTGACTC | |
| hIL12RB1 | CGGCTGACCCTGAAAGAG | Roche UPL probe #78 |
| | CAGCCCTTGACAGCCTTC | |
| hIL12RB2 | TCCAGATCCAGCAAATAGCA | Roche UPL probe #82 |
| | GTCCAAGGGCAGCTGTGT | |

Enzyme-linked immunosorbent assays (ELISAs). IL-21 was detected in culture supernatants using the mouse IL-21 DuoSet ELISA (R&D Systems) according to the manufacturer's instructions. To measure the relative amounts of TNP-specific antibodies in mouse serum, plates were coated with 5 μg/ml TNP₂-BSA or TNP₂₈-BSA (Biosearch Technology) overnight at 4° C. TNP-specific ELISA was otherwise performed as previously described (Roes and Rajewsky, 1993). To standardize and quantify relative amounts of TNP-specific Ig responses, all experimental samples were compared with a standardized dilution of pooled serum obtained from IL-27ra^{-/-} mice immunized with TNP-OVA. This standard was given the arbitrary concentration of 100 ug/ml. In the case of IgM, an anti-TNP monoclonal (BD Biosciences; clone G155-228) was used as a standard control.

Flow cytometric analysis. Cells were treated with Fc blocking Abs (anti-CD16/32 2.4G2) and then surface stained with the given markers. To measure cell viability, cells were stained with FITC-conjugated annexin V and 7-AAD according to manufacturers instructions (BD). Viable cells exclude both stains. IL-21 expression in human T cells was assessed by intracellular staining using Alexa 647 labeled anti-human IL-21 (eBiosciences; clone 3A3-N2). Samples were analyzed using a FACSCanto II or LSR II (Becton Dickinson) and data analyzed using Flow Jo software (Tree Star, Inc.). Contour profiles are presented as 5% probability contours with outliers.

Immunization. T-dependent immunization: Groups of age and sex-matched mice were immunized with TNP₁₄-OVA (30 ug/mouse) or OVA (100 ug/mouse), as indicated, emulsified in 100 ul of complete Freund's adjuvant (CFA; SIGMA) by subcutaneous injection into the flank. Where a second immunization was required, this was performed 21 days after the initial injection using the same dose of antigen emulsified in Incomplete Freund's adjuvant (IFA; SIGMA) to a volume of 100 ul per mouse and injected subcutaneously into the alternate flank. T-independent immunization: Groups of six mice per genotype were immunized i.p. with 100 μg of TNP-aminoethylcarboxymethyl-Ficoll in PBS. Serum was harvested 5 days later. Human naïve CD4+ T cells were FACS purified from tonsil cell preparations based on CD4⁺CD45RA⁺CXCR5⁻ phenotype. Cells were labelled with CFSE and stimulated with T cell activation and expansion beads (Miltenyi Biotech) at a bead:cell ratio of 2:1 in the presence of either no additional cytokine, 20 ng/ml rhIL-12, 20 ng/ml rhIL-23 or 50 ng/ml rhIL-27 for 5 days.

Mice, cells and reagents. IL-27ra^{+/+} and IL-27ra^{-/-} (Chen et al., 2000) mice (C57BL/6 background, n>33), OT-II TCR Tg (C57BL6) and DO11.10 TCR transgenic/rag2 deficient mice (DO11.10tg.rag2^{-/-} on the BALB/c background) were bred in a pathogen free facility at either The Garvan Institute, Australia or Genentech Inc. USA. Stat1^{-/-} mice (129Sv/Ev background) and 129Sv/Ev control mice were purchased from Taconic Transgenics, USA. The triple congenic mice (TCM), Igha_B6—CD45.1_Cross-B6.SJL were bred in a pathogen free facility at Genentech Inc. USA. The μMT (B6.129S2-Igh-6tm1Cgn/J mice) and rag2^{-/-} animals were purchased from Jackson Laboratories, Maine. All live animal experiments were approved by the Institutional Animal Care and Use Committee of Genentech or The Garvan/St. Vincent's Animal Experimentation Ethics Committee. Human PBMC buffy coats were obtained from the Red Cross, Australia and patients with the clinical diagnosis of HIES were recruited from Immunology Clinics in Canberra and Sydney, Australia. Unless otherwise indicated, all cytokines were purchased from R&D Systems, and all antibodies were from BD Biosciences. Cycloheximide was purchased from SIGMA-Aldrich.

Immunohistochemical analysis. Spleens were fixed in 10% formalin and subsequently embedded in paraffin. To detect GC, 5 gm sections were stained with biotin-conjugated PNA followed by visualisation with HRP-linked streptavidin and diamino benzidine (DAB). The sections were counterstained with Giemsa. Slide scanning and image analysis software were used to quantitate the percentage of each spleen that was positive for PNA staining.

Isolation of lymphocyte subsets. Unless otherwise indicated, primary mouse CD4⁺ T cells were isolated by magnetic depletion using MACS kits (Miltenyi Biotech) according to the manufacturer's instructions. The purity ranged from 90-95%. Where indicated, a FACSAria was used to obtain specific cell populations of >99% purity. After immunization, splenic leukocyte populations were isolated as follows: CD4⁺ (CD4⁺B220⁻); TFH(CD4⁺CXCR5⁺PD1⁺); non-GC B (B220⁺CD38⁺); GC B)(B220⁺CD38^{lo}; FDC (B220⁻CD35^{hi}CD32⁺) CD11b⁺ (CD11b⁺CD11c⁻B220⁻); CD11c⁺ (CD11c⁺CD11b⁻). For cell culture systems that required antigen presenting cells, splenocyte samples were magnetically depleted of T cells with anti-CD90 (Thy-1.2) MACS Micro Beads (Miltenyi Biotech) and irradiated (2,600 rads).

In vitro T cell stimulation. Primary mouse cells were cultivated in Iscove's modified Dulbecco's medium (Invitrogen) supplemented with 10% (volume/volume) heat-inactivated FBS (HyClone PerBio), 1 mM L-glutamine, 1% (volume/volume) penicillin and streptomycin (Invitrogen) and 55 uM 2-mercaptoethanol (MP Biomedicals). Splenic CD4+ T cells from C57BL/6, IL-27ra^{-/-}, 129 or Stat1^{-/-} mice were activated for the indicated times in plates coated with 5 μg/ml of anti-CD3 and in the presence of 1 μg/ml anti-CD28. Unfractionated DO11.10tg.rag2^{-/-} or OTIItg splenocytes were stimulated with OVA₃₂₃₋₃₃₉ peptide at the indicated concentrations. For T cell polarization the following combinations of blocking antibodies (all 5 ug/ml) and recombinant cytokines were used: (prefixes: m, murine; rh, recombinant human; rm, recombinant murine): N (no cytokine addition or blockade), T_{H}0 (hamster anti-mIFNγ, H22, rat anti-mIL-4, BVD4-1D11, and rat anti-mIL-12, C15.6), T_{H}1 (rat anti-mIL-4; 3.5 ng/ml rmIL-12), T_{H}2 (hamster anti-mIFNγ and rat anti-mIL-12; 3.5 ng/ml rmIL-4), T_{H}17 (hamster anti-mIFN™, rat anti-mIL-4, rat anti-mIL12, 5 ng/ml rmIL-6, 1 ng/ml rhTGFB1) and in the presence or absence of rmIL-27 (20 ng/ml). Human naïve CD4⁺ T cells were FACS purified from tonsil cell or PBMC preparations based on CD4⁺CD45RA⁺CXCR5⁻ phenotype. Cells were labeled with CFSE and stimulated with T cell activation and expansion beads (Miltenyi Biotech) at a bead:cell ratio of 2:1 in the presence of either no additional cytokine, 20 ng/ml rhIL-12, 20 ng/ml rhIL-23 or 50 ng/ml rhIL-27 for 5 days.

Bone marrow chimeras. BM chimeras were generated using two methods. Congenic C57BL6ptprca (CD45.1) or triple congenic mice (CD45.2, Thy1.1) were lethally irradiated (gamma source, 1150 rad) and reconstituted with equal numbers of BM cells from IL-27ra^{+/+} (CD45.1, Thy1.2) and IL-27ra^{-/-} (CD45.2, Thy1.2) mice by i.v. injection. Alternatively C57BL6.Rag2^{-/-} mice were lethally irradiated (600 rad) and reconstituted with either μMT and IL-27ra^{+/+}BM (1:1) or μMT and IL-27ra^{-/-}BM (1:1). After 8 weeks of reconstitution, the mice were bled to assess reconstitution by flow cytometry and immunized as described above.

Statistical analysis. Data was analyzed with Prism software to calculate unpaired, two-way Student's t-test.

### LITERATURE CITED

Awasthi, A., Carrier, Y., Peron, J. P., Bettelli, E., Kamanaka, M., Flavell, R. A., Kuchroo, V. K., Oukka, M., and Weiner, H. L. (2007). A dominant function for interleukin 27 in generating interleukin 10-producing anti-inflammatory T cells. Nat Immunol 8, 1380-1389.
Banerjee, S., Webber, C., and Poole, A. R. (1992). The induction of arthritis in mice by the cartilage proteoglycan aggrecan: roles of CD4+ and CD8+ T cells. Cell Immunol 144, 347-357.
Batten, M., and Ghilardi, N. (2007). The biology and therapeutic potential of interleukin 27. J Mol Med 85, 661-672.
Batten, M., Kljavin, N. M., L1, J., Walter, M. J., de Sauvage, F. J., and Ghilardi, N. (2008). Cutting edge: IL-27 is a potent inducer of IL-10 but not FoxP3 in murine T cells. J Immunol 180, 2752-2756.
Batten, M., Li, J., Yi, S., Kljavin, N. M., Danilenko, D. M., Lucas, S., Lee, J., de Sauvage, F. J., and Ghilardi, N. (2006). Interleukin 27 limits autoimmune encephalomyelitis by suppressing the development of interleukin 17-producing T cells. Nat Immunol 7, 929-936.
Cao, Y., Doodes, P. D., Glant, T. T., and Finnegan, A. (2008). IL-27 induces a Th1 immune response and susceptibility to experimental arthritis. J Immunol 180, 922-930.
Chen, Q., Ghilardi, N., Wang, H., Baker, T., Xie, M. H., Gurney, A., Grewal, I. S., and de Sauvage, F. J. (2000). Development of Th1-type immune responses requires the type I cytokine receptor TCCR. Nature 407, 916-920.
Collison, L. W., Workman, C. J., Kuo, T. T., Boyd, K., Wang, Y., Vignali, K. M., Cross, R., Sehy, D., Blumberg, R. S., and Vignali, D. A. (2007). The inhibitory cytokine IL-35 contributes to regulatory T-cell function. Nature 450, 566-569.
de Vinuesa, C. G., Cook, M. C., Ball, J., Drew, M., Sunners, Y., Cascalho, M., Wabl, M., Klaus, G. G., and MacLennan, I. C. (2000). Germinal centers without T cells. J Exp Med 191, 485-494.
Eddahri, F., Denanglaire, S., Bureau, F., Spolski, R., Leonard, W. J., Leo, O., and Andris, F. (2008). Interleukin-6/STAT3 signalling regulates the ability of naive T cells to acquire B cell help capacities. Blood.
Fazilleau, N., McHeyzer-Williams, L. J., Rosen, H., and McHeyzer-Williams, M. G. (2009). The function of follicular helper T cells is regulated by the strength of T cell antigen receptor binding. Nat Immunol 10, 375-384.
Fitzgerald, D. C., Zhang, G. X., El-Behi, M., Fonseca-Kelly, Z., Li, H., Yu, S., Saris, C. J., Gran, B., Ciric, B., and Rostami, A. (2007). Suppression of autoimmune inflammation of the central nervous system by interleukin 10 secreted by interleukin 27-stimulated T cells. Nat Immunol 8, 1372-1379.
Hamel, K., Doodes, P., Cao, Y., Wang, Y., Martinson, J., Dunn, R., Kehry, M. R., Farkas, B., and Finnegan, A. (2008). Suppression of proteoglycan-induced arthritis by anti-CD20 B Cell depletion therapy is mediated by reduction in autoantibodies and CD4+ T cell reactivity. J Immunol 180, 4994-5003.
Harris, M. B., Chang, C. C., Berton, M. T., Danial, N. N., Zhang, J., Kuehner, D., Ye, B. H., Kvatyuk, M., Pandolfi, P. P., Cattoretti, G., et al. (1999). Transcriptional repression of Stat6-dependent interleukin-4-induced genes by BCL-6: specific regulation of iepsilon transcription and immunoglobulin E switching. Mol Cell Biol 19, 7264-7275.
Hasan, M., Lopez-Herrera, G., Blomberg, K. E., Lindvall, J. M., Berglof, A., Smith, C. I., and Vargas, L. (2008). Defective Toll-like receptor 9-mediated cytokine production in B cells from Bruton's tyrosine kinase-deficient mice. Immunology 123, 239-249.
Johnston, R. J., Poholek, A. C., DiToro, D., Yusuf, I., Eto, D., Barnett, B., Dent, A. L., Craft, J., and Crotty, S. (2009). Bcl6 and Blimp-1 are reciprocal and antagonistic regulators of T follicular helper cell differentiation. Science 325, 1006-1010.
King, C. (2009). New insights into the differentiation and function of T follicular helper cells. Nat Rev Immunol. 2009 November; 9(11):757-66., 757-766.
Kopf, M., Herren, S., Wiles, M. V., Pepys, M. B., and Kosco-Vilbois, M. H. (1998). Interleukin 6 influences germinal center development and antibody production via a contribution of C3 complement component. J Exp Med 188, 1895-1906.
Korn, T., Bettelli, E., Gao, W., Awasthi, A., Jager, A., Strom, T. B., Oukka, M., and Kuchroo, V. K. (2007). IL-21 initiates an alternative pathway to induce proinflammatory T(H)17 cells. Nature 448, 484-487.
Larousserie, F., Charlot, P., Bardel, E., Froger, J., Kastelein, R. A., and Devergne, O. (2006). Differential effects of IL-27 on human B cell subsets. J Immunol 176, 5890-5897.
Lim, H. W., and Kim, C. H. (2007). Loss of IL-7 receptor alpha on CD4+ T cells defines terminally differentiated B cell-helping effector T cells in a B cell-rich lymphoid tissue. J Immunol 179, 7448-7456.
Marinova, E., Han, S., and Zheng, B. (2006). Human germinal center T cells are unique Th cells with high propensity for apoptosis induction. Int Immunol 18, 1337-1345.
Miyazaki, Y., Inoue, H., Matsumura, M., Matsumoto, K., Nakano, T., Tsuda, M., Hamano, S., Yoshimura, A., and Yoshida, H. (2005). Exacerbation of experimental allergic asthma by augmented Th2 responses in WSX-1-deficient mice. J Immunol 175, 2401-2407.
Niedbala, W., Wei, X. Q., Cai, B., Hueber, A. J., Leung, B. P., McInnes, I. B., and Liew, F. Y. (2007). IL-35 is a novel cytokine with therapeutic effects against collagen-induced arthritis through the expansion of regulatory T cells and suppression of Th17 cells. Eur J Immunol 37, 3021-3029.
Nurieva, R., Yang, X. O., Martinez, G., Zhang, Y., Panopoulos, A. D., Ma, L., Schluns, K., Tian, Q., Watowich, S. S., Jetten, A. M., and Dong, C. (2007). Essential autocrine regulation by IL-21 in the generation of inflammatory T cells. Nature 448, 480-483.
Nurieva, R. I., Chung, Y., Hwang, D., Yang, X. O., Kang, H. S., Ma, L., Wang, Y. H., Watowich, S. S., Jetten, A. M., Tian, Q., and Dong, C. (2008). Generation of T follicular helper cells is mediated by interleukin-21 but independent of T helper 1, 2, or 17 cell lineages. Immunity 29, 138-149.
Nurieva, R. I., Chung, Y., Martinez, G. J., Yang, X. O., Tanaka, S., Matskevitch, T. D., Wang, Y. H., and Dong, C. (2009). Bcl6 mediates the development of T follicular helper cells. Science 325, 1001-1005.
O'Neill, S. K., Cao, Y., Hamel, K. M., Doodes, P. D., Hutas, G., and Finnegan, A. (2007). Expression of CD80/86 on B cells is essential for autoreactive T cell activation and the development of arthritis. J Immunol 179, 5109-5116.
Pflanz, S., Hibbert, L., Mattson, J., Rosales, R., Vaisberg, E., Bazan, J. F., Phillips, J. H., McClanahan, T. K., de Waal Malefyt, R., and Kastelein, R. A. (2004). WSX-1 and glycoprotein 130 constitute a signal-transducing receptor for IL-27. J Immunol 172, 2225-2231.
Pflanz, S., Timans, J. C., Cheung, J., Rosales, R., Kanzler, H., Gilbert, J., Hibbert, L., Churakova, T., Travis, M., Vaisberg, E., et al. (2002). IL-27, a heterodimeric cytokine composed of EBI3 and p28 protein, induces proliferation of naive CD4(+) T cells. Immunity 16, 779-790.
Pot, C., Jin, H., Awasthi, A., Liu, S. M., Lai, C. Y., Madan, R., Sharpe, A. H., Karp, C. L., Miaw, S. C., Ho, I. C, Kuchroo, V. K. (2009) IL-27 induces the transcription factor c-Maf, cytokine IL-21, and the costimulatory receptor ICOS that coordinately act together to promote differentiation of IL-10-producing Trl cells. J. Immunol. 183(2), 797-801.
Remoli, M. E., Gafa, V., Giacomini, E., Severa, M., Lande, R., and Coccia, E. M. (2007). IFN-beta modulates the response to TLR stimulation in human DC: involvement of IFN regulatory factor-1 (IRF-1) in IL-27 gene expression. Eur J Immunol 37, 3499-3508.
Roes, J., and Rajewsky, K. (1993). Immunoglobulin D (IgD)-deficient mice reveal an auxiliary receptor function for IgD in antigen-mediated recruitment of B cells. J Exp Med 177, 45-55.
Schmitt, N., Morita, R., Bourdery, L., Bentebibel, S. E., Zurawski, S. M., Banchereau, J., and Ueno, H. (2009). Human dendritic cells induce the differentiation of interleukin-21-producing T follicular helper-like cells through interleukin-12. Immunity 31, 158-169.
Shimizu, S., Sugiyama, N., Masutani, K., Sadanaga, A., Miyazaki, Y., Inoue, Y., Akahoshi, M., Katafuchi, R., Hirakata, H., Harada, M., et al. (2005): Membranous glomerulonephritis development with Th2-type immune deviations in MRUlpr mice deficient for IL-27 receptor (WSX-1). J Immunol 175, 7185-7192.
Stumhofer, J. S., Laurence, A., Wilson, E. H., Huang, E., Tato, C. M., Johnson, L. M., Villarino, A. V., Huang, Q., Yoshimura, A., Sehy, D., et al. (2006). Interleukin 27 negatively regulates the development of interleukin 17-producing T helper cells during chronic inflammation of the central nervous system. Nat Immunol 7, 937-945.
Stumhofer, J. S., Silver, J. S., Laurence, A., Porrett, P. M., Harris, T. H., Turka, L. A., Ernst, M., Saris, C. J., O'Shea, J. J., and Hunter, C. A. (2007). Interleukins 27 and 6 induce STAT3-mediated T cell production of interleukin 10. Nat Immunol 8, 1363-1371.
Vogelzang, A., McGuire, H. M., Yu, D., Sprent, J., Mackay, C. R., and King, C. (2008). A fundamental role for interleukin-21 in the generation of T follicular helper cells. Immunity 29, 127-137.
Wei, L., Laurence, A., Elias, K. M., and O'Shea, J. J. (2007). IL-21 is produced by Th17 cells and drives IL-17 production in a STAT3-dependent manner. J Biol Chem 282, 34605-34610.
Yoshimoto, T., Okada, K., Morishima, N., Kamiya, S., Owaki, T., Asakawa, M., Iwakura, Y., Fukai, F., and Mizuguchi, J. (2004). Induction of IgG2a class switching in B cells by IL-27. J Immunol 173, 2479-2485.
Yu, D., Batten, M., Mackay, C. R., and King, C. (2009a). Lineage specification and heterogeneity of T follicular helper cells. Curr Opin Immunol.
Yu, D., Rao, S., Tsai, L. M., Lee, S. K., He, Y., Sutcliffe, E. L., Srivastava, M., Linterman, M., Zheng, L., Simpson, N., et al. (2009b). The transcriptional repressor Bcl-6 directs T follicular helper cell lineage commitment. Immunity 31, 457-468.
Zhou, L., Ivanov, II, Spolslci, R., Min, R., Shenderov, K., Egawa, T., Levy, D. E., Leonard, W. J., and Littman, D. R. (2007). IL-6 programs T(H)-17 cell differentiation by promoting sequential engagement of the IL-21 and IL-23 pathways. Nat Immunol 8, 967-974. The foregoing written description is considered to be sufficient to enable one of ordinary skill in the art to practice the invention. The present invention is not to be limited in scope by the example presented herein. Indeed, various modifications of the invention in addition to those shown and described herein will become apparent to those skilled in the art from the foregoing description and fall within the scope of the appended claims.

What is claimed is:

1. A method for treating or preventing lupus in an individual comprising administering to the individual an effective amount of an IL-27 antagonist.

2. The method of claim 1, wherein the IL-27 antagonist reduces the number of T follicular helper cells.

3. The method of claim 1, wherein the IL-27 antagonist reduces IL-21 expression in T follicular helper cells.

4. The method of claim 1, wherein the IL-27 antagonist reduces high affinity antigen-specific antibodies.

5. The method of claim 1, wherein the individual is a human.

6. The method of claim 1, wherein the individual has lupus.

7. The method of claim 1, wherein the individual has increased expression of one or more marker genes shown in FIG. 19A in peripheral blood mononuclear cells (PBMCs) from the individual as compared to a reference level.

8. The method of claim 7, wherein the expression of one or more marker genes is measured at the level of an RNA transcript or at the level of a protein expression.

9. The method of claim 7, wherein the reference level is determined based on the expression level of the corresponding marker gene in PBMCs from one or more healthy individuals.

10. The method of claim 1, wherein the IL-27 antagonist is an anti-IL-27 antibody that specifically binds to IL-27.

11. The method of claim 10, wherein the IL-27 antagonist is an anti-IL-27 antibody that specifically binds to the p28 subunit of IL-27 (“IL-27p28”).

12. The method of claim 10, wherein the IL-27 antagonist is an anti-IL-27 antibody that specifically binds to the Epstein Barr virus induced protein 3 (Ebi3) subunit of IL-27 (“IL-27Ebi3”).

13. The method of claim 10, wherein the anti-IL-27 antibody inhibits IL-27 signal transduction.

14. The method of claim 13, wherein the anti-IL-27 antibody inhibits IL-10 production.

15. The method of claim 13, wherein the anti-IL-27 antibody inhibits IL-21 production.

16. The method of claim 13, wherein the anti-IL-27 antibody is a monoclonal antibody.

17. The method of claim 13, wherein the anti-IL-27 antibody is an antibody fragment selected from the group consisting of Fab, Fab′-SH, Fv, scFv, and (Fab′)₂ fragments.

18. The method of claim 13, wherein the anti-IL-27 antibody is a humanized antibody.

19. The method of claim 13, wherein the anti-IL-27 antibody is a human antibody.

20. The method of claim 13, wherein the anti-IL-27 antibody is a bispecific antibody.

21. The method of claim 1, wherein the IL-27 antagonist is an anti-IL-27Ra antibody that specifically binds to IL-27Ra.

22. The method of claim 21, wherein the anti-IL-27Ra antibody is a monoclonal antibody.

23. The method of claim 21, wherein the anti-IL-27Ra antibody is an antibody fragment selected from the group consisting of Fab, Fab′-SH, Fv, scFv, and (Fab′)₂ fragments.

24. The method of claim 21, wherein the anti-IL-27Ra antibody is a humanized antibody.

25. The method of claim 21, wherein the anti-IL-27Ra antibody is a human antibody.

26. The method of claim 1, wherein the IL-27 antagonist is a small molecule that inhibits binding between IL-27 and its receptor.

27. The method of claim 1, wherein the IL-27 antagonist is a polypeptide that inhibits binding between IL-27 and its receptor.

28. The method of claim 1, wherein the IL-27 antagonist is a DNA or RNA aptamer that inhibits binding between IL-27 and its receptor.

29. The method of claim 1, wherein the IL-27 antagonist is a short interfering RNA that inhibits expression of IL-27, IL-27p28, IL-27Ebi3, or IL-27Ra.

30. The method of claim 1, wherein the IL-27 antagonist is administered intravenously, intramuscularly, subcutaneously, topically, orally, transdermally, intraperitoneally, intraorbitally, by implantation, by inhalation, intrathecally, intraventricularly, or intranasally.

31. An article of manufacture comprising an IL-27 antagonist and instructions for using the IL-27 antagonist to treat or prevent lupus in an individual.

32. A method for determining if a patient having lupus is likely to respond to an IL-27 antagonist treatment, comprising the steps of:
(a) measuring the expression level of a marker gene shown in FIG. 19A in a sample comprising peripheral blood mononuclear cells (PBMCs) obtained from the patient; and
(b) comparing the expression level measured in step (a) to a reference level, wherein an increase in the expression level as compared to the reference level indicates that the individual is likely to respond to the IL-27 antagonist treatment.

33. The method of claim 32, wherein the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or twenty one marker genes shown in FIG. 19A is measured and compared to the reference level of the respective genes.

34. The method of claim 32, wherein the expression level is measured at the level of an RNA transcript or at the level of a protein expression.

35. The method of claim 32, wherein the reference level is determined based on the expression level of the marker gene in PBMCs from one or more healthy individuals.

36. A method of preparing an expression profile for a patient having lupus, comprising the steps of:
(a) measuring the expression level of a marker gene shown in FIG. 19A in a sample comprising peripheral blood mononuclear cells (PBMCs) obtained from the patient;
(b) comparing the expression level measured in step (a) to a reference level; and
(c) generating a report summarizing the expression level measured in step (a) and the comparison determined in step (b).

37. The method of claim 36, wherein the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or twenty one marker genes shown in FIG. 19A is measured and compared to the reference level of the respective genes.

38. The method of claim 36, wherein the expression level is measured at the level of an RNA transcript or at the level of a protein expression.

39. The method of claim 36, wherein the reference level is determined based on the expression level of the marker gene in PBMCs from one or more healthy individuals.

40. The method of claim 36, wherein the report includes a recommendation for an IL-27 antagonist treatment for the patient.

41. A kit comprising reagents for measuring the expression level of one or more marker genes shown in FIG. 19A in a sample comprising PBMCs from an individual having lupus.

42. The kit of claim 47, wherein the reagents comprise polynucleotides capable of specifically hybridizing to one or more marker genes shown in FIG. 19A or complements of said genes.

43. The kit of claim 42, wherein the polynucleotides are capable of specifically hybridizing to at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or all marker genes shown in FIG. 19A or complements of said genes.

44. The kit of claim 42, wherein the polynucleotides are provided as an array, a gene chip, or gene set.

45. The kit of claim 41, wherein the reagents comprise at least a pair of primers and a probe for detecting the expression level of a marker gene shown in FIG. 19A by PCR.

46. The kit of claim 41, further comprising instructions for assessing if the individual having lupus is likely to respond to an IL-27 antagonist treatment.
